(19) United States
(12) Patent Application Publication
(10) Pub. No.: US 2020/0191798 A1
(43) Pub. Date: Jun. 18, 2020

(54) DOSE DETERMINATION FOR IMMUNOTHERAPEUTIC AGENTS

(71) Applicant: BIONTECH RNA PHARMACEUTICALS GMBH, Mainz (DE)

(72) Inventors: Stefan Josef Christof Friedrich Strobl, Mainz (DE); Roman Peter Rösemann, Mainz (DE); Ugur Sahin, Mainz (DE); Veronika Jahndel, Mainz (DE); Doreen Schwarck-Kokarakis, Mainz (DE); Yves Hüsemann, Mainz (DE); Kathrin Dorer, Mainz (DE); Robert Jabulowsky, Mainz (DE)

(21) Appl. No.: 16/343,624

(22) PCT Filed: Oct. 25, 2017
(86) PCT No.: PCT/EP2017/077293

§ 371 Date: Apr. 19, 2019

(30) Foreign Application Priority Data
Oct. 25, 2016 (EP) PCT/EP2016/075647

(51) Int. Cl. G01N 33/68; A61K 31/7105; G01N 33/50

(52) U.S. Cl. CPC G01N 33/6866; A61K 31/7105; A61K 45/06; G01N 33/6854; G01N 33/5047

(57) ABSTRACT

The present invention relates to methods for determining suitable doses for administration of immunotherapeutic compounds, whose effectiveness and toxicity can vary at the same dose between individuals due to natural variations within individual subjects, such as variations in the reaction of the immune system in response to administration of such immunotherapeutic compounds.

### TECHNICAL FIELD OF THE INVENTION

The present invention relates to methods for determining suitable doses for immunotherapeutic compounds, whose effectiveness and toxicity can significantly vary at the same dose between individuals due to natural variations in individual subjects, such as variations in the reaction of the immune system in response to administration of such immunotherapeutic compounds.

### BACKGROUND OF THE INVENTION

Normally, therapeutically effective doses for therapeutic compounds are easily determined. To determine an efficacious amount of a therapeutic compound, such as an antibiotic or analgesic, increasing amounts of the therapeutic compound are given to a cohort of subjects until the desired therapeutic effect is observed, with the expectation that the greater the dose, the greater the therapeutic effect. However, one factor limiting the dose of a therapeutic compound that can be given is the appearance of an unwanted side effect observed with administration of higher, yet still therapeutic, amounts of the compound. Such amounts, or toxic doses, are also easily determined by simply giving greater and greater amounts of the compound until a side effect is observed, such as fever, nausea or more severe effects such as organ failure, shock, etc. Since such therapeutically effective and toxic doses are normally determined on a per kilogram body weight basis or normalized against some other variable with respect to the individual, doses for therapeutic compounds that are both efficacious and non-toxic are easily extrapolated for any patient.

However, there are situations in which effective therapeutic and/or toxic doses and/or their extrapolation to all individuals cannot be determined using standard methods, such as where a therapeutic agent does not meet the expectation that the higher the amount administered, the higher the therapeutic effect observed. The present inventors have observed that, in the context of administering an immunotherapeutic agent that is a Toll-like receptor agonist, the same dose administered to different individuals leads to different results, for example, different levels of therapeutic effect and/or different levels of side effects. The present inventors have also observed that cells isolated from different individuals respond differently in terms of cell activation as measured by cytokine expression to the same amount of an immunotherapeutic agent. The present invention is based, in part, on these observations, which indicate that the immunological reaction to immunotherapeutic agents is subject to natural variation in the immune system of individuals such that there is no universal dose amount based on a per unit basis, e.g., weight, surface area, for all individuals that guarantees an acceptable therapeutic effect for a particular immunotherapeutic agent, and preferably provides an acceptable toxicity profile.

### SUMMARY OF THE INVENTION

The present invention relates to methods for determining a suitable dose of an immunotherapeutic agent, whose amount is preferably both therapeutic and non-toxic for that individual. Not wanting to be limited to a particular mechanism, it is believed that immunotherapeutic agents, e.g., RNA-based molecules, in order to provide a therapeutic effect, are dependent on many native factors whose activities and amounts vary among individuals, thus leading to variable effects, both therapeutic and unwanted, observed in different individuals when the same agent is administered at the same dose.

In particular, the present invention relates to a method for determining a suitable dose of an immunotherapeutic agent for administration to an individual, comprising (a) separately contacting multiple different doses of the immunotherapeutic agent with immune-reactive material of the individual, and (b) measuring at least one immunological reaction caused by the multiple different doses of the immunotherapeutic agent. In an embodiment, step (b) is characterized by qualitatively and/or quantitatively measuring at least one immunological reaction, preferably quantitatively measuring at least one immunological reaction. A dose used in the methods of the present invention is an amount of an immunotherapeutic agent. The dose, e.g., can be in amounts of picograms, nanograms, micrograms, milligrams and grams or equivalents thereof in other unit systems. The dose, or amount, can be an absolute amount, i.e., the dose does not vary with regard to age, sex, weight, body mass index reflecting amounts of adipose tissue, surface area of the skin, etc. of an individual. Alternatively, the dose can take into account variations among individuals, such as age, sex, weight, body mass index reflecting amounts of adipose tissue, surface area of the skin, etc. The multiple different doses represent separate doses at different amounts, preferably the different amounts are quantified in the same way (expressed in the same unit), for example, all the multiple different doses are in units of mg per kg body weight or are all an absolute amount. In an embodiment where the step of contacting the multiple different doses takes place in vitro, the dose can be an absolute amount or can take into account the type of immune-reactive material, for example, where the immune-reactive material comprises immune cells, the dose can take into account the number of the immune cells or the number of a subtype of immune cells.

Any method known in the art for contacting the immunotherapeutic agent with the immune-reactive material of the individual or for measuring an immunological reaction can be employed for the purposes of the present invention. Exemplary embodiments include where the immune-reactive material is a cell composition comprising immune cells isolated from the individual, such as whole blood or a purified population of immune cells isolated from the individual. In embodiments where the multiple different doses of the immunotherapeutic agent are administered to the individual, the immune-reactive material is the immune system itself and the immunological reaction generated by the immune cells in the body of the individual is measured. For example, after administration of the immunotherapeutic agent, blood or lymph can be isolated from the individual and tested for the desired immunological reaction, such as the expression of a cytokine. In one embodiment where the immunotherapeutic agent is administered to the individual, such administration can be on the skin, i.e., a skin scratch or skin prick test.

In an embodiment, the method is carried out in vivo or is carried out in vitro, or at least one of the steps is carried out in vivo and other steps are carried out in vitro, e.g., the contacting step is carried out in vivo and the measuring of the immunological response takes place in vitro by, for example collecting blood from the individual and measuring an immunological reaction in the blood or in cells isolated from the blood. Preferably, all the steps in the method are carried out in vitro.

The immunotherapeutic agent useful in the methods of the invention is any agent, molecule, compound, composition, etc., that can effect a change in at least one component of the immune system of an animal, preferably a human. For example, an immunotherapeutic agent can activate a particular type of immune cell or can cause a particular type of immune cell to go quiescent, whose activation or quiescence can be measured, e.g., by a change in expression of a cytokine. Other effects on the at least one component of the immune system can include causing immune cells to proliferate or to differentiate, e.g., an increase or decrease in immune cell numbers in the blood. Moreover, the immunotherapeutic agent can induce the production of antibodies or can activate immune cells, such as cytotoxic T cells to induce a cytotoxic effect. Although changes in cytokine expression are useful as immunological reactions in the context of the methods of the invention as described below, in an embodiment cytokines, in view of their ability to effect changes to the immune system (cause an immunological reaction), also can be immunotherapeutic agents. Exemplary immunological reactions useful in the methods of the present invention are discussed infra.

In an embodiment, the immunotherapeutic agent is a compound that is an agonist of a Toll-like receptor (TLR), e.g., a TLR-1, TLR-2, TLR-3, TLR-4, TLR-5, TLR-6, TLR-7, TLR-8, TLR-9, TLR-10, TLR-11, TLR12 or TLR13 agonist. Preferably, the TLR is one that is located inside the cell, such as TLR-3, TLR-7, TLR-8 and TLR-9. Also preferably, the immunotherapeutic agent is an agonist of Toll-like receptor-7 (TLR-7) or Toll-like receptor-8 (TLR-8). TLR-7 agonists are known and include compounds such as single-stranded RNA molecules and imidazo-quinoline compounds, such as thiazoloquinolone, the antiviral compound imiquimod, and resiquimod. Other TLR-7 agonist compounds include N-(4-(4-amino-2-(2-methoxyethyl)-1H-imidazo[4,5-c]quinolin-1-yl)butyl)-N-(tetrahydro-2H-pyran-4-yl)acetamide, N-{4-[4-amino-2-(2-methoxyethyl)-1H-imidazo[4,5-c]quinolin-1-yl]butyl}-N-(1,1-dioxothietan-3-yl)acetamide, N-(4-(4-amino-2-ethyl-1H-imidazo[4,5-c]quinolin-1-yl)butyl)-N-(1,1-dioxidothietan-3-yl)acetamide, poly(dThymidine), the guanosine analog loxoribine, and bropirimine, as well as other nucleotide-base analogs. TLR-8 agonists also include single-stranded RNA molecules as well as 2-ethyl-1-(4-((2-methyltetrahydrofuran-3-yl)amino)butyl)-1H-imidazo[4,5-c]quinolin-4-amine and 1-(4-(cyclohexylamino)butyl)-2-ethyl-1H-imidazo[4,5-c]quinolin-4-amine. It has been observed that particles comprising protamine and RNA can activate TLR-7 when taken up by, for example, plasmacytoid dendritic cells or TLR-8 when taken up by, for example, monocytes (WO 2009/144230). In one embodiment, the immunotherapeutic agent can be a virus, e.g., an RNA virus. The immunotherapeutic agent preferably can be a nucleic acid molecule, such as a single-stranded RNA molecule or other RNA-based molecules, which nucleic acid molecule encodes an immunoreactive peptide or protein. More preferably, the immunotherapeutic agent whose immunological reaction is measured is a single-stranded RNA molecule which encodes one or more peptides, each peptide comprising an epitope that is specifically expressed on diseased cells or tissue, such as tumor tissue. Expression of these peptides (immunoreactive peptides) from the RNA results in their presentation on the cell surface in a complex with MHC molecules and ultimately the induction of an immune response against diseased cells or tissue expressing the epitopes. In a preferred embodiment, the RNA molecule may be complexed with cationic lipids, cationic polymers and other substances with positive charges that can form complexes with negatively charged nucleic acids. Additional exemplary immunotherapeutic agents are described infra.

As used herein, immune-reactive material useful in the methods of the invention includes all or a part of the immune system of an individual for which a change in some characteristic can be measured (the immunological reaction) when contacted with an immunotherapeutic agent. Preferably, the immune-reactive material comprises a cell of the immune system, e.g., an immune cell or an immunoreactive cell or a composition comprising the immune cell or immunoreactive cell, such as whole blood or lymph fluid. The immune cells also can be substantially purified, e.g., 80%, 85%, 90%, 95%, 99% pure. The term “immune cells” refers to cells of the immune system involved in defending the body of an individual. The term “immune cells” encompasses specific types of immune cells and their precursors including leucocytes comprising macrophages, monocytes (precursors of macrophages), granulocytes such as neutrophils, eosinophils and basophils, dendritic cells, mast cells, and lymphocytes such as B cells, T cells and natural killer (NK) cells. Macrophages, monocytes (precursors of macrophages), neutrophils, dendritic cells, and mast cells are phagocytic cells. In an embodiment, the immune-reactive material of the individual comprises cells isolated from the blood of the individual or the immune-reactive material comprises whole blood isolated from the individual or the immune-reactive material comprises lymph fluid isolated from the individual. In an embodiment where the method is carried out in vitro, the immune-reactive material of the individual comprises or essentially consist of peripheral blood mononuclear cells (PBMCs), or where the immune-reactive material is whole blood, the whole blood can be optionally supplemented with dendritic cells, such as plasmacytoid dendritic cells (pDCs) and/or monocyte-derived immature dendritic cells (iDCs). The dendritic cells can be from a heterologous or syngeneic source or can be autologous, preferably autologous. Since an immunotherapeutic agent can be an immune cell, in an embodiment of the invention, the immune-reactive material and the immunotherapeutic agent can both be immune cells.

As used herein, an immunological reaction in the context of the methods of the invention is a change in a measurable characteristic of the immune system or a component of the immune system, and is preferably one which is known to indicate a therapeutic effect due to administration of an immunotherapeutic agent. For example, an immunological reaction includes a change in the activity of an immune cell, which activity can be a change in the differentiation phenotype of the immune cell or a change in the proliferative ability of the immune cell, or a change in the expression or amount of one or more cytokines produced by the immune cell, either on a nucleic acid or protein level. An immunological reaction can be a change in the amount of a specific type of immune cell in an individual, such as lymphocytes or T cells. An immunological reaction also can be a change in platelet counts or platelet activation kinetics in an individual. The immunological reaction also can be a change in the inflammatory state of the individual, such as an inflammatory reaction on the skin, e.g., contact dermatitis. An immunological reaction can also include the induction of an immune response against a target antigen such as the induction of a cytotoxic T cell response against the antigen. Preferably, the immunological reaction that is measured is a change in the amount/concentration of one or more cytokines secreted by the immune cells measured, e.g., by detecting the cytokine itself or detecting the nucleic acid encoding the cytokine.

Cytokines are a broad category of small proteins that are important in cell signaling in that they are released by cells and affect the behavior of other cells, although cytokines also can be involved in autocrine signaling. Cytokines are produced by a broad range of cells, including a broad range of immune cells, as well as endothelial cells, fibroblasts, and various stromal cells, and a given cytokine may be produced by more than one type of cell. Exemplary cytokines include monokines, lymphokines, interleukines or chemokines, e.g., IL-1, IL-2, IL-3, IL-4, IL-5, IL-6, IL-7, IL-8, IL-9, IL-10, IL-12, IL-15, IL-21, INF-α, INF-γ, GM-CSF. In an embodiment, the cytokine is involved in regulating lymphoid homeostasis, preferably a cytokine which is involved in and preferably induces or enhances development, priming, expansion, differentiation and/or survival of T cells. In one embodiment, the cytokine is an interleukin. In a preferred embodiment, the cytokine is one or more of the following: interleukin-6 (IL-6), interleukin-12 (IL-12), tumor necrosis factor-alpha (TNF-α), interferon-alpha (IFN-α) such as interferon-alpha 2a (IFN-α2a), interferon-gamma (IFN-γ), interferon-gamma-induced protein (IP10), interleukin 1-beta (IL-1β), interleukin 2 (IL-2), interleukin 12p70 (IL-12p70).

Interleukin 1-beta (IL-1β) is a member of the interleukin 1 family of cytokines, which is produced by activated macrophages as a proprotein, which is proteolytically processed to its active form by caspase 1 (CASP1/ICE). This cytokine is an important mediator of the inflammatory response, and is involved in a variety of cellular activities, including cell proliferation, differentiation, and apoptosis. The induction of cyclooxygenase-2 (PTGS2/COX2) by this cytokine in the central nervous system (CNS) is found to contribute to inflammatory pain hypersensitivity.

Interleukin-2 (IL-2) is a protein that regulates the activities of white blood cells (leukocytes, often lymphocytes) that are responsible for immunity. IL-2 is part of the body's natural response to microbial infection, and in discriminating between foreign (“non-self”) and “self”. IL-2 has key roles in key functions of the immune system, e.g., tolerance and immunity, primarily via its direct effects on T cells. In the thymus, where T cells mature, it prevents autoimmune diseases by promoting the differentiation of certain immature T cells into regulatory T cells, which suppress other T cells that are otherwise primed to attack normal healthy cells in the body. IL-2 also promotes the differentiation of T cells into effector T cells and into memory T cells when the initial T cell is also stimulated by an antigen, thus helping the body fight off infections.

Interleukin 6 (IL-6) acts as both a pro-inflammatory cytokine and an anti-inflammatory myokine and is secreted by T cells and macrophages to stimulate immune responses, e.g., during infection and after trauma, especially burns or other tissue damage leading to inflammation. IL-6 also plays a role in fighting infection, since IL-6 has been shown in mice to be required for resistance against bacterium Streptococcus pneumoniae. The role of IL-6 as an anti-inflammatory cytokine is mediated through its inhibitory effects on TNF-α and IL-1, and through activation of IL-1RA and IL-10.

Interleukin 12 (IL-12) is naturally produced by dendritic cells, macrophages, neutrophils, and human B-lymphoblastoid cells in response to antigenic stimulation. IL-12 is a heterodimeric cytokine encoded by two separate genes, IL-12A (p35) and IL-12B (p40). The active heterodimer (referred to as ‘p70’), and a homodimer of p40 are formed following protein synthesis. IL-12 is involved in the differentiation of naive T cells into Th1 cells and is known as a T cell-stimulating factor, which can stimulate the growth and function of T cells. It stimulates the production of interferon-gamma (IFN-γ) and tumor necrosis factor-alpha (TNF-α) from T cells and natural killer (NK) cells, mediates enhancement of the cytotoxic activity of NK cells and CD8+ cytotoxic T lymphocytes, and also has anti-angiogenic activity.

Tumor necrosis factor-alpha (TNF-α) (cachexin or cachectin) is involved in systemic inflammation and is one of the cytokines that make up the acute phase reaction. It is produced chiefly by activated macrophages, although it can be produced by many other cell types such as dendritic cells, monocytes, CD4+ lymphocytes, NK cells, neutrophils, mast cells, eosinophils, and neurons. The primary role of TNF-α is in the regulation of immune cells. TNF-a, being an endogenous pyrogen, is able to induce fever, apoptotic cell death, cachexia, inflammation and to inhibit tumorigenesis and viral replication and respond to sepsis via IL-1 and IL-6 producing cells.

Human type I interferons (IFNs) belong to a large subgroup of interferon proteins that help regulate the activity of the immune system. The mammalian types are designated IFN-α (alpha), IFN-β (beta), IFN-κ (kappa), IFN-δ (delta), IFN-E (epsilon), IFN-τ (tau), IFN-w (omega), and IFN-ζ (zeta, also known as limitin). They are mainly involved in innate immune responses against viral infection. The genes responsible for their synthesis come in 13 subtypes that are called IFNA1, IFNA2, IFNA4, IFNA5, IFNA6, IFNA7, IFNA8, IFNA10, IFNA13, IFNA14, IFNA16, IFNA17, IFNA21. These genes are found together in a cluster on chromosome 9. The IFN-β proteins have antiviral activity that is involved mainly in innate immune response. Two types of IFN-β have been described, IFN-β1 (IFNB1) and IFN-β3 (IFNB3). IFN-α and IFN-β are secreted by many cell types including dendritic cells, lymphocytes (NK cells, B-cells and T-cells), macrophages, fibroblasts, endothelial cells, osteoblasts and others. They stimulate both macrophages and NK cells to elicit an anti-viral response, and are also active against tumors. Plasmacytoid dendritic cells have been identified as being the most potent producers of type I IFNs in response to Toll-like receptor (TLR) activation, e.g., TLR-7, 8 and/or 9, and have thus been called natural IFN producing cells.

Interferon gamma (IFN-γ) is a dimerized soluble cytokine that is the only member of the type II class of interferons. IFN-γ is critical for innate and adaptive immunity against viral, some bacterial and protozoal infections. IFN-γ is an important activator of macrophages and inducer of Class II major histocompatibility complex (MHC) molecule expression. Aberrant IFN-γ expression is associated with a number of autoinflammatory and autoimmune diseases. The importance of IFN-γ in the immune system stems in part from its ability to inhibit viral replication directly, and most importantly from its immunostimulatory and immunomodulatory effects. IFN-γ is produced predominantly by macrophages, natural killer (NK) and natural killer T cells (NKT) as part of the innate immune response, and by CD4+Th1 and CD8+ cytotoxic T lymphocyte effector T cells (CTL) once antigen-specific immunity develops.

Interferon-gamma-induced protein 10 (IP-10), also known as C-X-C motif chemokine 10 (CXCL10) or small-inducible cytokine B10, is a small cytokine belonging to the CXC chemokine family, which is secreted by several cell types, e.g., in response to IFN-γ. These cell types include macrophages, dendritic cells, monocytes, endothelial cells and fibroblasts. IP-10 has been attributed to several roles, such as chemoattraction for monocytes/macrophages, T cells, NK cells, and dendritic cells, promotion of T cell adhesion to endothelial cells, antitumor activity, and inhibition of bone marrow colony formation and angiogenesis.

In an embodiment, the measuring of an immunological reaction involves the use of labeled ligands which specifically bind to a molecule, e.g., a labeled nucleic acid probe that hybridizes to a nucleic acid and/or a labeled antibody or fragment/derivative thereof that specifically binds to a peptide, such as a cytokine.

According to the invention, measuring the presence or amount of a nucleic acid can be carried out using known nucleic acid detection methods such as methods involving hybridization or nucleic acid amplification techniques. In one embodiment, mRNA transcripts are detected or the quantity thereof is determined using RT-PCR or Northern blot analysis. Such nucleic acid detection methods may involve the use of oligonucleotides hybridizing to the nucleic acids. Suitable oligonucleotides typically vary in length from five to several hundred nucleotides, more typically about 20-70 nucleotides in length or shorter, even more typically about 10-30 nucleotides in length.

According to the invention, measuring the presence or amount of a peptide, such as a cytokine, can be carried out in a number of ways including, but not limited to, immune-detection using an antibody binding specifically to the peptide. Methods for using antibodies to detect peptides are well known and include ELISA, competitive binding assays, and the like. In general, such assays use an antibody or antibody fragment that specifically binds the peptide directly or indirectly bound to a label that provides for detection, e.g. indicator enzymes, radiolabels, fluorophores, or paramagnetic particles.

According to the invention, where the immunological reaction is detected by measuring cell growth or a lack thereof, or a change in the differentiation state of a cell, such measuring can be carried out in a number of ways including, but not limited to, the counting of the number of cells, or measuring ³H uptake into cellular DNA to determine cellular proliferation. A change in differentiation can be measured by observing a change in expression of a cellular protein associated with a particular differentiation state or by observing a change in the visual phenotype of the cell associated with a particular differentiation state. Additional methods are known in the art and can be readily used in the methods herein.

In an embodiment of the method, at least one immunological reaction is measured, or 2 or more immunological reactions are measured, or 3, 4, 5, 6, 7, 8, 9, 10 or more immunological reactions are measured.

In certain embodiments of the method, the multiple different doses of the immunotherapeutic agent can be two, three, four, five, six, seven, eight, nine, ten or more than ten different doses. Further, the multiple different doses of the immunotherapeutic agent can represent a dose escalation, preferably a linear or logarithmic dose escalation, for example, 1, 2, 3, 4, 5, etc., or 1, 3, 9, 27, 81, 243, etc. or 0.1, 1, 10, 100, 1000, etc. In one embodiment, steps (a) and (b) are performed sequentially. Preferably, step (b) is performed 2 to 48 hours after step (a), preferably 4 to 24 hours after step (a).

In one preferred embodiment, step (a) of the method (separately contacting multiple different doses of the immunotherapeutic agent with immune-reactive material of the individual) is carried out in vivo and is characterized by separately contacting multiple different doses of the immunotherapeutic agent with immune-reactive material of the individual in separate administration steps, each characterized by administration of one dose of the immunotherapeutic agent to the individual. The separate administration steps can be carried out subsequently and are separated from each other by time intervals of between 2 and 30 days, such as between 7 and 28 days, preferably separated by 7 days, 14 days, 21 days or 28 days, more preferably separated by 7 days or 14 days. Preferably, measuring the at least one immunological reaction is separately carried out following each separate administration step. In one embodiment, the separate administration steps can be carried out at substantially the same time, for example, by administering the multiple different doses to the skin at substantially the same time. In this embodiment, the immunological reaction measured, for example, can be contact demiatitis that can be detected visually.

Many types of immunotherapeutic agents have been given to patients to provide for a therapeutic effect, and in view of such knowledge, standard doses or standard dose ranges for such types of immunotherapeutic agents that provide a therapeutic effect are known. Where such a standard dose or range of doses is known, the multiple different doses contacted in step (a) will preferably include a dose that is below the standard dose or range and/or a dose that lies within the standard dose range and/or a dose that is greater than the standard dose or range. For example, where the standard dose range of an RNA molecule given as a cancer vaccine to an individual is between 5 and 100 μg, then exemplary multiple different doses contacted in step (a) can be 2 μg, 10 μg, and 150 μg. Where a standard dose or range of doses is not known for a particular type of immunotherapeutic, the known dose or range of doses for a similar type of immunotherapeutic can be employed in the methods of the invention, or a standard dose or range of doses can be empirically determined and then applied according to the invention for determining a suitable dose of an immunotherapeutic agent for an individual.

In an embodiment, the multiple different doses comprise at least one dose that is below the standard dose range for the immunotherapeutic agent. In an embodiment, the multiple different doses comprise at least one dose that lies within the standard dose range for the immunotherapeutic agent. In an embodiment, the first of the separate administration steps is characterized by administration of a dose of the immunotherapeutic agent that is below the standard dose for the immunotherapeutic agent, and wherein a dose administered in the subsequent of the separate administration steps is optionally higher than the dose administered in the first of the separate administration steps.

In an embodiment where the contacting step takes place in vitro, the standard dose or range of doses for an immunotherapeutic agent is one that is known to be equivalent to the standard dose for the same immunotherapeutic agent in vivo. Such equivalencies are known in the art or can be determined using methods known in the art. In one embodiment, the standard dose can be the same as that when administered (contacted) in vivo, but adjusted, for example, by the amount of immune-reactive material used in vitro (e.g., number of immune cells) and/or by the volume of the immune-reactive material as compared to contacting in vivo. In an embodiment, a standard dose for contacting in vitro is the same as the amount/concentration of the immunotherapeutic agent per ml of blood or lymph or per number of a specific type of immune cell that is observed in the individual when the known standard dose is administered to the individual. In an embodiment, a standard “in vitro” dose is equivalent to the concentration of the immunotherapeutic agent achieved in whole blood when a standard “in vivo” dose is administered to the individual. For example, when a standard dose of 1 mg/kg results in a concentration of the immunotherapeutic agent of 10 μg/ml in whole blood, then the equivalent standard dose in vitro of the 1 mg/kg in vivo standard dose is 10 μg/ml where the immune-reactive material is whole blood.

The methods of the present invention can further comprise a step for detecting the presence or absence of at least one unwanted reaction, such as an unwanted immunological reaction, e.g., a too high or too low level of expression of a cytokine, or side effect or adverse event or reaction, such as organ toxicity resulting from contacting an immunotherapeutic agent with immune-reactive material, e.g., resulting from administration of the immunotherapeutic agent to the individual. This step can be carried out following each step of contacting an immunotherapeutic agent with immune-reactive material, whether in vitro or in vivo. In one embodiment, where the therapeutic effect to be achieved is a reduction in the amount of a specific type of cell, an unwanted reaction can be increased expression of a cytokine which increased expression is known to lessen the ability of the immunotherapeutic agent to be therapeutic. Side effects are a subset of unwanted reactions which can be detected when the contacting step is carried out in vivo and are those unwanted reactions that reflect some level of discomfort to the individual, and which can vary in severity. A more severe side effect is termed herein a non-tolerable side effect. Exemplary side effects include, but are not limited to paresthesia, fatigue, headache, muscular pain, pressure on chest or pain in chest, shivering, elevated temperature or fever, tinnitus, joint pain, dizziness, sweating, hypotonia and/or tachycardia. Exemplary non-tolerable side effects can be those that are life-threatening to the individual, such as systemic inflammatory response syndrome that can ultimately lead to organ failure.

In an embodiment, where at least one side effect is detected following administration of one of the multiple different doses, all subsequent doses may be administered with at least one antitoxic agent. In an embodiment, where at least one non-tolerable side effect is detected following administration of one of the multiple different doses, all subsequent doses will be administered with at least one antitoxic agent. Subsequent doses may be identical or lower to the dose which caused the tolerable or non-tolerable side effect which triggered administration of at least one antitoxic agent. If a dose causes tolerable or non-tolerable side effects in the absence of an antitoxic agent, but is tolerated when administered in the presence of an antitoxic agent, the following doses may be higher but may only be administered in the presence of an antitoxic agent. The antitoxic agent is preferably an anti-pyretic drug, such as an NSAID, e.g., ibuprofen, naproxen, ketoprofen, and nimesulide; aspirin and related salicylates such as choline salicylate, magnesium salicylate, and sodium salicylate; paracetamol (acetaminophen); metamizole; nabumetone; and phenazone.

In an embodiment where at least one side effect is detected following administration of one of the multiple different doses that is not administered with at least one antitoxic agent, the next dose of the immunotherapeutic agent to be subsequently administered is identical to or less than the dose administered in the previous administration step. Preferably, where the at least one unwanted reaction is a non-tolerable side effect, the next dose of the immunotherapeutic agent to be subsequently administered is less than the dose, administered in the previous administration step. The subsequent administration step that directly follows the previous administration step can be further followed by one or more further administration steps that optionally represent a dose escalation scheme from step to step.

In an embodiment where at least one side effect is detected following administration of one of the multiple different doses that is administered with at least one antitoxic agent, the next dose of the immunotherapeutic agent to be subsequently administered is less than the dose administered in the previous administration step.

In an embodiment, where no side effect is detected following administration of any of the multiple different doses, a dose where the at least one immunological reaction indicates an acceptable therapeutic effect reflects a suitable dose for administration of the immunotherapeutic agent to the individual. In an embodiment where more than one dose is determined to be a suitable dose, a dose where the at least one immunological reaction provides the strongest indication of an acceptable therapeutic effect is the dose of the immunotherapeutic agent that is administered to the individual. The strongest indication of an acceptable therapeutic effect will depend on the immunological reaction being measured. For example, the strongest indication can be where the expression of a cytokine is the greatest or the least observed among the multiple different doses administered. The highest dose administered that provides an acceptable therapeutic effect is not necessarily the same dose at which the strongest indication of an acceptable therapeutic effect is provided.

In an embodiment where at least one side effect is detected following administration of any of the multiple different doses, a dose in a subsequent administration step that is administered with at least one antitoxic agent and where the at least one immunological reaction indicates an acceptable therapeutic effect for the immunotherapeutic agent reflects a suitable dose for administration of the immunotherapeutic agent to the individual. In an aspect of this embodiment where no side effect is detected following administration of any of the multiple different doses administered with at least one antitoxic agent, a dose where the at least one immunological reaction provides the strongest indication of an acceptable therapeutic effect is a suitable dose for administration of the immunotherapeutic agent to the individual. This aspect concerns the situation where there are multiple different doses that are suitable doses since no side effects are detected at these doses administered with an antitoxic agent. The strongest indication of an acceptable therapeutic effect will depend on the immunological reaction being measured. For example, the strongest indication can be where the expression of a cytokine is the greatest or the least observed among the multiple different doses administered with the at least one antitoxic agent. The highest dose administered with the at least one antitoxic agent at which no side effect is detected is not necessarily the same dose at which the strongest indication of an acceptable therapeutic effect is provided. In another aspect of this embodiment, where at least one side effect is detected following administration of any of the multiple different doses administered with at least one antitoxic agent, the highest dose where the side effect is not detected or is least severe or is otherwise deemed acceptable in light of the severity of disease is a suitable dose for administration of the immunotherapeutic agent to the individual. This aspect concerns the situation where some of the doses administered with the at least one antitoxic agent result in side effects or where side effects are detected with all doses administered with at least one antitoxic agent. Thus, a suitable dose is one which is therapeutically effective and where no side effects are detected or where the side effects are least severe or where the side effects are otherwise deemed acceptable in light of the severity of disease.

According to the present invention, a dose determined to be a suitable dose for a particular immunotherapeutic agent for a particular individual is a dose where the at least one immunological reaction that is known to indicate an acceptable, preferably optimal, therapeutic effect in the individual for that immunotherapeutic agent reflects a suitable dose for administration of the immunotherapeutic agent to the individual. Preferably, a suitable dose also results in a minimum of unwanted reactions or side effects in the individual, whether or not administered with at least one antitoxic agent. In an embodiment where contacting the immunotherapeutic agent with immune-reactive material occurs in vivo, the dose where the at least one immunological reaction indicates an acceptable therapeutic effect reflects a suitable dose directly, i.e., the suitable dose for administration of the immunotherapeutic agent to the individual will be the same or similar to one or more of the multiple different doses contacted with the immune-reactive material. In an embodiment where contacting the immunotherapeutic agent with immune-reactive material occurs in vitro, the multiple different doses used in the in vitro method are not necessarily the same as those which would be administered to the individual that would provide for an acceptable therapeutic effect. Thus, a dose where at least one immunological reaction indicates an acceptable therapeutic effect in vitro can reflect a suitable dose indirectly. The relationship between the dose contacted in vitro and its equivalent in vivo dose is either known or can be determined using methods known in the art. For example, where the dose of an immunotherapeutic agent contacted in vitro is an amount relative to the number of immune cells being contacted (e.g., 10 ng/10⁸ cells), the equivalent in vivo dose is a dose that results in the same or similar amount of the immunotherapeutic agent in the blood relative to the same number of the same immune cells (10 ng/10⁸ of the same cells).

The therapeutic effect will depend on the therapeutic effect expected to be provided by the immunotherapeutic agent. In one embodiment, an acceptable therapeutic effect includes, but is not limited to, the arresting or slowing down of the progression of the disease; inhibiting or slowing the development of a new disease in an individual; decreasing the frequency or severity of symptoms and/or recurrences in an individual who currently has or who previously has had a disease; and/or prolonging, i.e., increasing, the lifespan of the individual. In one embodiment, an exemplary optimal therapeutic effect is one in which the disease has been eliminated such that no further treatment is required. In an embodiment where the disease is cancer, an acceptable therapeutic effect is one in which at least the size and/or number of tumors are not increased, and preferably is one in which the size and/or number of tumors are decreased and an optimal therapeutic effect would be the complete disappearance of any and all tumors and where no further administration of the immunotherapeutic agent is required.

Where it has been determined that at least one immunological reaction or a specific change in at least one immunological reaction indicates an acceptable therapeutic effect, a dose which results in the same reaction or change thereof indicates that the dose is a suitable dose for providing an acceptable therapeutic effect. Further, the strength or weakness of the at least one immunological reaction or change thereof also can indicate the strength or weakness of the therapeutic effect at that dose. The correlation between at least one immunological reaction and a therapeutic effect for many immunotherapeutic agents is known. Moreover, such correlations can be determined using methods known in the art. For example, at least one immunological reaction can be measured in one or more individuals that have been administered an immunotherapeutic agent at a dose which resulted in a therapeutic effect, preferably in an acceptable therapeutic effect, and the at least one immunological reaction that is consistently observed among the individuals is indicative of a therapeutic effect, preferably an acceptable therapeutic effect, for that immunotherapeutic agent. For example, where a dose of an immunotherapeutic agent resulting in an acceptable therapeutic effect consistently correlates with an increase in the expression of three different cytokines and the differentiation of a certain type of immune cell into a more mature immune cell, the increase in the expression of three different cytokines and the differentiation of the immune cell are immunological reactions indicative of an acceptable therapeutic effect. In such a manner, a set of immunological reactions or “set of parameters” which are indicative of a therapeutic effect for any immunotherapeutic agent can be determined. In an embodiment of the invention, where the known set of immunological reactions that indicate an acceptable therapeutic effect for the immunotherapeutic agent is the same or substantially the same as that observed in an individual administered a dose of the immunotherapeutic agent, the administered dose reflects a suitable dose for administration of the immunotherapeutic agent to the individual.

Exemplary embodiments of the invention include, but are not limited to those (i) where the acceptable therapeutic effect for a particular immunotherapeutic agent is known to be indicated by the highest level of expression of a particular cytokine, a suitable dose for an individual is reflected by a dose which results in the highest level of expression of that particular cytokine, or (ii) where the acceptable therapeutic effect for a particular immunotherapeutic agent is known to be indicated by the lowest level of expression of a particular cytokine, a suitable dose for an individual is reflected by a dose which results in the lowest level of expression of that particular cytokine, or (iii) where the acceptable therapeutic effect for a particular immunotherapeutic agent is known to be indicated by the induction of expression of a particular cytokine, a suitable dose for an individual is reflected by a dose which results in the induction of expression of that particular cytokine, or (iv) where the acceptable therapeutic effect for a particular immunotherapeutic agent is known to be indicated by a specific expression pattern of multiple cytokines, a suitable dose for an individual is reflected by a dose which results in substantially the same specific expression pattern of the multiple cytokines, or (v) where the acceptable therapeutic effect for a particular immunotherapeutic agent is known to be indicated by the induction of differentiation of a particular immune cell, a suitable dose for an individual is reflected by a dose which results in the same or similar induction of differentiation of the particular immune cell, or (vi) where the acceptable therapeutic effect for a particular immunotherapeutic agent is known to be indicated by the induction or enhancement of an effector function of an immune cell, e.g. a T cell, a suitable dose for an individual is reflected by a dose which results in the same or similar induction or enhancement of the effector function of the immune cell.

The present invention further relates to a method of treating an individual with a suitable dose of an immunotherapeutic agent comprising administering to the individual a dose of the immunotherapeutic agent that has been determined to be suitable according to the methods of the invention. Preferably, the immunotherapeutic agent is a TLR agonist. In an embodiment, the method of treating an individual with a suitable dose of an immunotherapeutic agent comprises (a) separately contacting multiple different doses of the immunotherapeutic agent with immune-reactive material of the individual, (b) measuring at least one immunological reaction caused by the multiple different doses of the immunotherapeutic agent, wherein a dose where the at least one immunological reaction indicates an acceptable therapeutic effect for the immunotherapeutic agent reflects a suitable dose for administration to the individual, and (c) administering the immunotherapeutic agent to the individual at the suitable dose. In an embodiment, the method of treating an individual with a suitable dose of an immunotherapeutic agent comprises administering the immunotherapeutic agent to the individual at a suitable dose, wherein the suitable dose is determined by (a) separately contacting multiple different doses of the immunotherapeutic agent with immune-reactive material of the individual, and (b) measuring at least one immunological reaction caused by the multiple different doses of the immunotherapeutic agent, wherein a dose where the at least one immunological reaction indicates an acceptable therapeutic effect for the immunotherapeutic agent reflects a suitable dose for administration to the individual.

In an embodiment, the suitable dose of the immunotherapeutic agent is administered with at least one antitoxic agent. This embodiment is where the suitable dose of the immunotherapeutic agent is a dose at which at least one side effect is detected without administration of at least one antitoxic agent.

In a preferred embodiment, the method is an immunotherapy method for treating cancer and the immunotherapeutic agent is a nucleic acid, preferably single-stranded RNA encoding one or more epitopes that are expressed specifically on the cancer cells. Preferably, the one or more epitopes are neoepitopes.

Other features and advantages of the instant invention will be apparent from the following detailed description and claims.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., (1995) Helvetica Chimica Acta, CH-4010 Basel, Switzerland.

The practice of the present invention will employ, unless otherwise indicated, conventional methods of biochemistry, cell biology, immunology, and recombinant DNA techniques which are explained in the literature in the field (cf., e.g., Molecular Cloning: A Laboratory Manual, 4th Edition, M. R. Green, J. Sambrook et al. eds., Cold Spring Harbor Laboratory Press, Cold Spring Harbor 2012).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e., the subject-matter consists in the inclusion of a stated member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context.

The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The present invention envisions the immunotherapy of a disease by administering a suitable dose of an immunotherapeutic agent, which suitable dose is determined by the methods described herein. Since the effectiveness of an immunotherapeutic agent will depend on natural variations among individuals with regard to their immune system, a suitable therapeutically effective and preferably non-toxic dose needs to be determined individually for each patient. In a preferred embodiment, the immunotherapeutic agent is for treating cancer. In a preferred embodiment, the immunotherapy can be effected by active immunotherapeutic methods.

The invention specifically is directed to the determination of a suitable dose of an immunotherapeutic agent for an individual. Once such a suitable dose has been identified, the immunotherapeutic agent can be administered to the individual at that dose in order to induce an immunological reaction, such as an immune response against a specific target. In a preferred embodiment, the immune response is inducing and/or activating appropriate effector cells such as T cells that recognize epitopes expressed on tumor cells via an appropriate antigen receptor, such as a T cell receptor or artificial T cell receptor, resulting in the death of the diseased cell expressing the epitope.

Immunotherapeutic approaches encompassed within the invention include immunization with a peptide or polypeptide containing an epitope, ii) nucleic acid encoding the peptide or polypeptide containing an epitope, and iii) recombinant viruses encoding the peptide or polypeptide containing an epitope.

Dendritic cells (DCs) are leukocyte populations that present antigens captured in peripheral tissues to T cells via both MHC class II and I antigen presentation pathways. It is well known that DCs are potent inducers of immune responses and the activation of these cells is a critical step for the induction of antitumoral immunity. Dendritic cells are conveniently categorized as “immature” and “mature” cells, which can be used as a simple way to discriminate between two well characterized phenotypes. However, this nomenclature should not be construed to exclude all possible intermediate stages of differentiation. Immature dendritic cells are characterized as antigen presenting cells with a high capacity for antigen uptake and processing, which correlates with the high expression of Fey receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1 BB). DC maturation is referred to as the status of DC activation at which such antigen-presenting DCs leads to T-cell priming, while its presentation by immature DCs results in tolerance. DC maturation is chiefly caused by biomolecules with microbial features (bacterial DNA, viral RNA, endotoxin, etc.) detected by innate receptors (Pattern Recognition Receptors) pro-inflammatory cytokines (TNF, IL-1, IFNs), ligation of CD40 on the DC surface by CD40L, and substances released from cells undergoing stressful cell death. The DCs can be derived by culturing bone marrow cells, as well as cells derived from buffy coats or whole blood, in vitro with cytokines, such as granulocyte-macrophage colony-stimulating factor (GM-CSF) and IL-4.

A preferred immunotherapeutic agent is an immunoreactive peptide or a nucleic acid encoding one or more such peptides, which peptide can comprise an epitope, preferably a neoepitope resulting from a disease-specific mutation. The term “disease-specific mutation” in the context of the present invention relates to a somatic mutation that is present in the nucleic acid of a diseased cell but absent in the nucleic acid of a corresponding normal, not diseased cell. The disease can be cancer, thus, the term “tumor-specific mutation” or “cancer-specific mutation” relate to a somatic mutation that is present in the nucleic acid of a tumor or cancer cell but absent in the nucleic acid of a corresponding normal, i.e. non-tumorous or non-cancerous, cell. The terms “tumor-specific mutation” and “tumor mutation” and the terms “cancer-specific mutation” and “cancer mutation” are used interchangeably herein.

In embodiments where the immunotherapeutic agent is an antigen or a nucleic acid encoding said antigen, a “cellular immune response”, a “cellular response”, a “cellular response against an antigen” or a similar term is meant to include a cellular immunological response directed to cells characterized by presentation of an antigen with class I or class II MHC. The cellular response relates to cells called T cells or T-lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill diseased cells such as cancer cells, preventing the production of more diseased cells. Preferably, an anti-tumor CTL response is stimulated against tumor cells expressing one or more tumor expressed antigens and preferably presenting such tumor expressed antigens with class I MHC.

An “antigen” according to the invention covers any substance, preferably a peptide or protein, which is a target of and/or induces an immune response such as a specific reaction with antibodies or T-lymphocytes (T cells). Preferably, an antigen comprises at least one epitope such as a T cell epitope. Preferably, an antigen in the context of the present invention is a molecule which, optionally after processing, induces an immune reaction, which is preferably specific for the antigen (including cells expressing the antigen). The antigen or a T cell epitope thereof is preferably presented by a cell, preferably by an antigen presenting cell which includes a diseased cell, in particular a cancer cell, in the context of MHC molecules, which results in an immune response against the antigen (including cells expressing the antigen). An antigen is preferably a product which corresponds to or is derived from a naturally occurring antigen. Such naturally occurring antigens may include or may be derived from allergens, viruses, bacteria, fungi, parasites and other infectious agents and pathogens or an antigen may also be a tumor antigen. According to the present invention, an antigen may correspond to a naturally occurring product, for example, a viral protein, or a part thereof. In preferred embodiments, the antigen is a surface polypeptide, i.e., a polypeptide naturally displayed on the surface of a cell, a pathogen, a bacterium, a virus, a fungus, a parasite, an allergen, or a tumor. The antigen may elicit an immune response against a cell, a pathogen, a bacterium, a virus, a fungus, a parasite, an allergen, or a tumor.

The term “disease-associated antigen” or “disease-specific antigen” is used in it broadest sense to refer to any antigen associated with or specific to a disease. Such an antigen is a molecule which contains epitopes that will stimulate a host's immune system to make a cellular antigen-specific immune response and/or a humoral antibody response against the disease. The disease-associated antigen may therefore be used for therapeutic purposes. Disease-associated antigens are preferably associated with infection by microbes, typically microbial antigens, or associated with cancer, typically tumors.

The term “pathogen” refers to pathogenic biological material capable of causing disease in an organism, preferably a vertebrate organism. Pathogens include microorganisms such as bacteria, unicellular eukaryotic organisms (protozoa), fungi, as well as viruses.

In the context of the present invention, the term “tumor antigen” or “tumor-associated antigen” relates to proteins that are under normal conditions specifically expressed in a limited number of tissues and/or organs or in specific developmental stages, for example, the tumor antigen may be under normal conditions specifically expressed in stomach tissue, preferably in the gastric mucosa, in reproductive organs, e.g., in testis, in trophoblastic tissue, e.g., in placenta, or in germ line cells, and are expressed or aberrantly expressed in one or more tumor or cancer tissues. In this context, “a limited number” preferably means not more than 3, more preferably not more than 2. The tumor antigens in the context of the present invention include, for example, differentiation antigens, preferably cell type specific differentiation antigens, i.e., proteins that are under normal conditions specifically expressed in a certain cell type at a certain differentiation stage, cancer/testis antigens, i.e., proteins that are under normal conditions specifically expressed in testis and sometimes in placenta, and germ line specific antigens. In the context of the present invention, the tumor antigen is preferably associated with the cell surface of a cancer cell and is preferably not or only rarely expressed in normal tissues. Preferably, the tumor antigen or the aberrant expression of the tumor antigen identifies cancer cells. In the context of the present invention, the tumor antigen that is expressed by a cancer cell in a subject, e.g., a patient suffering from a cancer disease, can be a self-protein or a non-self-protein. In preferred embodiments, the tumor antigen in the context of the present invention is expressed under normal conditions specifically in cancerous tissue or in a tissue or organ that is non-essential, i.e., tissues or organs which when damaged by the immune system do not lead to death of the subject, or in organs or structures of the body which are not or only hardly accessible by the immune system or are protected through a tolerance mechanism, e.g., through the presence of a high concentration of T_{reg} cells. The amino acid sequence of the tumor antigen can be identical between the tumor antigen which is expressed in normal tissues and the tumor antigen which is expressed in cancer tissues or the amino acid sequences can be different, for example, at only a single amino acid or at more than one amino acid, and preferably at more than 2, 3, 4, 5, 6, 7, 8, 9, or 10 amino acids. The terms “tumor antigen”, “tumor-expressed antigen”, “cancer antigen” and “cancer-expressed antigen” are equivalents and are used interchangeably herein.

The terms “epitope”, “antigen peptide”, “antigen epitope”, “immunogenic peptide” and “MHC binding peptide” are used interchangeably herein and refer to an antigenic determinant in a molecule such as an antigen, i.e., to a part in or fragment of an immunologically active compound that is recognized by the immune system, for example, that is recognized by a T cell, in particular when presented in the context of MHC molecules. An epitope of a protein preferably comprises a continuous or discontinuous portion of said protein and is preferably between 5 and 100, preferably between 5 and 50, more preferably between 8 and 30, most preferably between 10 and 25 amino acids in length, for example, the epitope may be preferably 8, 9, 10, 11, 12, 13, 14, 15, 16, 17, 18, 19, 20, 21, 22, 23, 24, or 25 amino acids in length. According to the invention, an epitope may bind to MHC molecules such as MHC molecules on the surface of a cell and thus, may be an “MHC binding peptide” or “antigen peptide”. The term “major histocompatibility complex” and the abbreviation “MHC” include MHC class I and MHC class II molecules and relate to a complex of genes which is present in all vertebrates. MHC proteins or molecules are important for signaling between lymphocytes and antigen presenting cells or diseased cells in immune reactions, wherein the MHC proteins or molecules bind peptides and present them for recognition by T cell receptors. The proteins encoded by the MHC are expressed on the surface of cells, and display both self-antigens (peptide fragments from the cell itself) and non-self-antigens (e.g., fragments of invading microorganisms) to a T cell. Preferred such immunogenic portions bind to an MHC class I or class II molecule. As used herein, an immunogenic portion is said to “bind to” an MHC class I or class II molecule if such binding is detectable using any assay known in the art. The term “MHC binding peptide” relates to a peptide which binds to an MHC class I and/or an MHC class II molecule. In the case of class I MHC/peptide complexes, the binding peptides are typically 8-10 amino acids long although longer or shorter peptides may be effective. In the case of class II MHC/peptide complexes, the binding peptides are typically 10-25 amino acids long and are in particular 13-18 amino acids long, whereas longer and shorter peptides may be effective.

As used herein the term “neoepitope” refers to an epitope that is not present in a reference such as a normal non-cancerous or germline cell but is found in diseased cells, such as cancer cells. This includes, in particular, situations wherein in a normal non-cancerous or germline cell a corresponding epitope is found, however, due to one or more mutations in a cancer cell the sequence of the epitope is changed so as to result in the neoepitope. Moreover, a neoepitope may not only be specific to the diseased cells but also can be specific to the patient having the disease.

In one particularly preferred embodiment of the invention, an epitope or neoepitope is a T cell epitope. As used herein, the term “T cell epitope” refers to a peptide which binds to a MHC molecule in a configuration recognized by a T cell receptor. Typically, T cell epitopes are presented on the surface of an antigen-presenting cell.

As used herein, the term “predicting immunogenic amino acid modifications” refers to a prediction whether a peptide comprising such amino acid modification will be immunogenic and thus useful as epitope, in particular T cell epitope, in vaccination.

According to the invention, a T cell epitope may be present in a vaccine as a part of a larger entity such as a vaccine sequence and/or a polypeptide comprising more than one T cell epitope. The presented peptide or T cell epitope is produced following suitable processing.

T cell epitopes may be modified at one or more residues that are not essential for TCR recognition or for binding to MHC. Such modified T cell epitopes may be considered immunologically equivalent.

Preferably a T cell epitope when presented by MHC and recognized by a T cell receptor is able to induce in the presence of appropriate co-stimulatory signals, clonal expansion of the T cell carrying the T cell receptor specifically recognizing the peptide/MHC-complex.

Preferably, a T cell epitope comprises an amino acid sequence substantially corresponding to the amino acid sequence of a fragment of an antigen. Preferably, said fragment of an antigen is an MHC class I and/or class II presented peptide.

A T cell epitope according to the invention preferably relates to a portion or fragment of an antigen which is capable of stimulating an immune response, preferably a cellular response against the antigen or cells characterized by expression of the antigen and preferably by presentation of the antigen such as diseased cells, in particular cancer cells. Preferably, a T cell epitope is capable of stimulating a cellular response against a cell characterized by presentation of an antigen with class I MHC and preferably is capable of stimulating an antigen-responsive cytotoxic T-lymphocyte (CTL).

In some embodiments the antigen is a self-antigen, particularly a tumor antigen. Tumor antigens and their determination are known to the skilled person.

The term “immunogenicity” relates to the relative effectivity to induce an immune response that is preferably associated with therapeutic treatments, such as treatments against cancers. As used herein, the term “immunogenic” relates to the property of having immunogenicity. For example, the term “immunogenic modification” when used in the context of a peptide, polypeptide or protein relates to the effectivity of said peptide, polypeptide or protein to induce an immune response that is caused by and/or directed against said modification. Preferably, the non-modified peptide, polypeptide or protein does not induce an immune response, induces a different immune response or induces a different level, preferably a lower level, of immune response.

According to the invention, the term “immunogenicity” or “immunogenic” preferably relates to the relative effectivity to induce a biologically relevant immune response, in particular an immune response which is useful for vaccination. Thus, an amino acid modification or modified peptide is immunogenic if it induces an immune response against the target modification in a subject, which immune response may be beneficial for therapeutic or prophylactic purposes.

“Antigen processing” or “processing” refers to the degradation of a polypeptide or antigen into procession products, which are fragments of said polypeptide or antigen (e.g., the degradation of a polypeptide into peptides) and the association of one or more of these fragments (e.g., via binding) with MHC molecules for presentation by cells, preferably antigen presenting cells, to specific T cells.

In an embodiment, an immunotherapeutic agent can comprise antigen presenting cells (APC), which are cells that present peptide fragments of protein antigens in association with MHC molecules on their cell surface. Some APCs may activate antigen specific T cells. Professional antigen-presenting cells are very efficient at internalizing antigen, either by phagocytosis, pinocytosis or by receptor-mediated endocytosis, and then displaying a fragment of the antigen, bound to a class II MHC molecule, on their membrane. The T cell recognizes and interacts with the antigen-class II MHC molecule complex on the membrane of the antigen-presenting cell. An additional co-stimulatory signal is then produced by the antigen-presenting cell, leading to activation of the T cell. The expression of co-stimulatory molecules is a defining feature of professional antigen-presenting cells. The main types of professional antigen-presenting cells are dendritic cells, which have the broadest range of antigen presentation, and are probably the most important antigen-presenting cells, macrophages, B-cells, and certain activated epithelial cells.

Non-professional antigen-presenting cells do not constitutively express the MHC class II proteins required for interaction with naive CD4⁺ T cells; these are expressed only upon stimulation of the non-professional antigen-presenting cells by certain cytokines such as IFNγ.

Antigen presenting cells can be loaded with MHC class I and class II presented peptides by transducing the cells with nucleic acid, preferably RNA, encoding a peptide or polypeptide comprising the peptide to be presented, e.g. a nucleic acid encoding the antigen.

In some embodiments, an immunotherapeutic agent of the invention comprising a gene delivery vehicle that targets a dendritic or other antigen presenting cell may be administered to a patient, resulting in transfection that occurs in vivo. In vivo transfection of dendritic cells, for example, may generally be performed using any methods known in the art, such as those described in WO 97/24447, or the gene gun approach described by Mahvi et al., Immunology and cell Biology 75:456-460, 1997.

The term “antigen presenting cell” also includes target cells.

“Target cell” shall mean a cell which is a target for an immune response such as a cellular immune response. Target cells include cells that present an antigen or an antigen epitope, i.e. a peptide fragment derived from an antigen, and include any undesirable cell such as a cancer cell. In preferred embodiments, the target cell is a cell expressing an antigen as described herein and preferably presenting said antigen with class I MHC.

The term “portion” refers to a fraction. With respect to a particular structure such as an amino acid sequence or protein the term “portion” thereof may designate a continuous or a discontinuous fraction of said structure. Preferably, a portion of an amino acid sequence comprises at least 1%, at least 5%, at least 10%, at least 20%, at least 30%, preferably at least 40%, preferably at least 50%, more preferably at least 60%, more preferably at least 70%, even more preferably at least 80%, and most preferably at least 90% of the amino acids of said amino acid sequence. Preferably, if the portion is a discontinuous fraction said discontinuous fraction is composed of 2, 3, 4, 5, 6, 7, 8, or more parts of a structure, each part being a continuous element of the structure. For example, a discontinuous fraction of an amino acid sequence may be composed of 2, 3, 4, 5, 6, 7, 8, or more, preferably not more than 4 parts of said amino acid sequence, wherein each part preferably comprises at least 5 continuous amino acids, at least 10 continuous amino acids, preferably at least 20 continuous amino acids, preferably at least 30 continuous amino acids of the amino acid sequence.

The terms “part” and “fragment” are used interchangeably herein and refer to a continuous element. For example, a part of a structure such as an amino acid sequence or protein refers to a continuous element of said structure.

A portion, a part or a fragment of a structure preferably comprises one or more functional properties of said structure. For example, a portion, a part or a fragment of an epitope, peptide or protein is preferably immunologically equivalent to the epitope, peptide or protein it is derived from. In the context of the present invention, a “part” of a structure such as an amino acid sequence preferably comprises, preferably consists of at least 10%, at least 20%, at least 30%, at least 40%, at least 50%, at least 60%, at least 70%, at least 80%, at least 85%, at least 90%, at least 92%, at least 94%, at least 96%, at least 98%, at least 99% of the entire structure or amino acid sequence.

In an embodiment, the immunotherapeutic agent can be an immunoreactive cell. An immunoreactive cell relates to an immune-reactive material in that it is a cell which exerts effector functions during an immune reaction. An immunoreactive cell preferably is capable of binding an antigen or a cell characterized by presentation of an antigen or an antigen peptide derived from an antigen and mediating an immune response. For example, such cells secrete cytokines and/or chemokines, secrete antibodies, recognize cancerous cells, and optionally eliminate such cells. For example, immunoreactive cells comprise T cells (cytotoxic T cells, helper T cells, tumor infiltrating T cells), B cells, natural killer cells, neutrophils, macrophages, and dendritic cells. Preferably, in the context of the present invention, “immunoreactive cells” are T cells, preferably CD4⁺ and/or CD8⁺ T cells. In an embodiment of the present invention, immune-reactive material of the individual preferably can comprise an immunoreactive cell or a composition comprising the immunoreactive cell.

An “immunoreactive cell” also can recognize an antigen or an antigen peptide derived from an antigen with some degree of specificity, in particular if presented in the context of MHC molecules such as on the surface of antigen presenting cells or diseased cells such as cancer cells. Preferably, said recognition enables the cell that recognizes an antigen or an antigen peptide derived from said antigen to be responsive or reactive. If the cell is a helper T cell (CD4⁺ T cell) bearing receptors that recognize an antigen or an antigen peptide derived from an antigen in the context of MHC class II molecules such responsiveness or reactivity may involve the release of cytokines and/or the activation of CD8⁺ lymphocytes (CTLs) and/or B-cells. If the cell is a CTL such responsiveness or reactivity may involve the elimination of cells presented in the context of MHC class I molecules, i.e., cells characterized by presentation of an antigen with class I MHC, for example, via apoptosis or perform-mediated cell lysis. CTL responsiveness may include sustained calcium flux, cell division, production of cytokines such as IFN-γ and TNF-α, up-regulation of activation markers such as CD44 and CD69, and specific cytolytic killing of antigen expressing target cells. CTL responsiveness may also be determined using an artificial reporter that accurately indicates CTL responsiveness. Such CTL that recognizes an antigen or an antigen peptide derived from an antigen and are responsive or reactive are also termed “antigen-responsive CTL” herein. If the cell is a B cell such responsiveness may involve the release of immunoglobulins.

The terms “T cell” and “T lymphocyte” are used interchangeably herein and include T helper cells (CD4+ T cells) and cytotoxic T cells (CTLs, CD8+ T cells) which comprise cytolytic T cells.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptor (TCR). The thymus is the principal organ responsible for the maturation of T cells. Several different subsets of T cells have been discovered, each with a distinct function.

T helper cells assist other white blood cells in immunologic processes, including differentiation of B cells into plasma cells and activation of cytotoxic T cells and macrophages, among other functions. These cells are also known as CD4+ T cells because they express the CD4 protein on their surface. Helper T cells become activated when they are presented with peptide antigens by MHC class II molecules that are expressed on the surface of antigen presenting cells (APCs) in the context of co-stimulation. Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8+ T cells since they express the CD8 glycoprotein on their surface. These cells recognize their targets by binding to antigen associated with MHC class I in the context of co-stimulation, which is present on the surface of every nucleated cell of the body.

A majority of T cells have a T cell receptor (TCR) existing as a complex of several proteins. The actual T cell receptor is composed of two separate peptide chains, which are produced from the independent T cell receptor alpha and beta (TCRα and TCRβ) genes and are called α- and (β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells.

According to the invention, the term “antigen receptor” includes naturally occurring receptors such as T cell receptor as well as engineered receptors, which confer an arbitrary specificity such as the specificity of a monoclonal antibody onto an immune effector cell such as a T cell. In this way, a large number of antigen-specific T cells can be generated for adoptive cell transfer. Thus, an antigen receptor according to the invention may be present on T cells, e.g. instead of or in addition to the T cell's own T cell receptor. Such T cells do not necessarily require processing and presentation of an antigen for recognition of the target cell but rather may recognize preferably with specificity any antigen present on a target cell. Preferably, said antigen receptor is expressed on the surface of the cells. For the purpose of the present invention, T cells comprising an antigen receptor are comprised by the term “T cell” as used herein. Specifically, according to the invention, the term “antigen receptor” includes artificial receptors comprising a single molecule or a complex of molecules which recognize, i.e. bind to, a target structure (e.g. an antigen) on a target cell such as a cancer cell (e.g. by binding of an antigen binding site or antigen binding domain to an antigen expressed on the surface of the target cell) and may confer specificity onto an immune effector cell such as a T cell expressing said antigen receptor on the cell surface. Preferably, recognition of the target structure by an antigen receptor results in activation of an immune effector cell expressing said antigen receptor. An antigen receptor may comprise one or more protein units said protein units comprising one or more domains as described herein. As used herein, an “antigen receptor” also may be a “chimeric antigen receptor (CAR)”, “chimeric T cell receptor” or “artificial T cell receptor”.

An antigen can be recognized by an antigen receptor through any antigen recognition domains (herein also referred to simply as “domains”) able to form an antigen binding site such as through antigen-binding portions of antibodies and T cell receptors which may reside on the same or different peptide chains. In one embodiment, the two domains forming an antigen binding site are derived from an immunoglobulin. In one embodiment, the two domains forming an antigen binding site are derived from a T cell receptor. Particularly preferred are antibody variable domains, such as single-chain variable fragments (scFv) derived from monoclonal antibodies and T cell receptor variable domains, in particular TCR alpha and beta single chains. In fact almost anything that binds a given target with high affinity can be used as an antigen recognition domain.

The first signal in activation of T cells is provided by binding of the T cell receptor to a short peptide presented by the major histocompatibility complex (MHC) on another cell. This ensures that only a T cell with a TCR specific to that peptide is activated. The partner cell is usually a professional antigen presenting cell (APC), usually a dendritic cell in the case of naïve responses, although B cells and macrophages can be important APCs. The peptides presented to CD8+ T cells by MHC class I molecules are typically 8-10 amino acids in length; the peptides presented to CD4+ T cells by MHC class II molecules are typically longer, as the ends of the binding cleft of the MHC class II molecule are open.

In the context of the present invention, a molecule is capable of binding to a target if it has a significant affinity for said predetermined target and binds to said predetermined target in standard assays. “Affinity” or “binding affinity” is often measured by equilibrium dissociation constant (K_{D}). A molecule is not (substantially) capable of binding to a target if it has no significant affinity for said target and does not bind significantly to said target in standard assays.

Cytotoxic T lymphocytes may be generated in vivo by incorporation of an antigen or an antigen peptide into antigen-presenting cells in vivo. The antigen or antigen peptide may be represented as protein, as DNA (e.g. within a vector) or as RNA. The antigen may be processed to produce a peptide partner for the MHC molecule, while a fragment thereof may be presented without the need for further processing. The latter is the case in particular, if these can bind to MHC molecules. In general, administration to a patient by intradermal injection is possible. However, injection may also be carried out intranodally into a lymph node (Maloy et al., 2001, Proc Natl Acad Sci USA 98:3299-303) or injected intravenously. Other modes of administration can include intramuscular and subcutaneous administration. The resulting cells present the complex of interest and are recognized by autologous cytotoxic T lymphocytes which then propagate.

Specific activation of CD4+ or CD8+ T cells may be detected in a variety of ways. Methods for detecting specific T cell activation include detecting the proliferation of T cells, the production of cytokines (e.g., lymphokines), or the generation of cytolytic activity. For CD4+ T cells, a preferred method for detecting specific T cell activation is the detection of the proliferation of T cells. For CD8+ T cells, a preferred method for detecting specific T cell activation is the detection of the generation of cytolytic activity.

By “cell characterized by presentation of an antigen” or “cell presenting an antigen” or similar expressions is meant a cell such as a diseased cell, e.g. a cancer cell, or an antigen presenting cell presenting the antigen it expresses or a fragment derived from said antigen, e.g. by processing of the antigen, in the context of MHC molecules, in particular MHC Class I molecules. Similarly, the terms “disease characterized by presentation of an antigen” denotes a disease involving cells characterized by presentation of an antigen, in particular with class I MHC. Presentation of an antigen by a cell may be effected by transfecting the cell with a nucleic acid such as RNA encoding the antigen.

By “fragment of an antigen which is presented” or similar expressions is meant that the fragment can be presented by MHC class I or class II, preferably MHC class I, e.g. when added directly to antigen presenting cells. In one embodiment, the fragment is a fragment which is naturally presented by cells expressing an antigen.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect such as induction of a humoral and/or cellular immune response, the strength and/or duration of the induced immune reaction, or the specificity of the induced immune reaction. In the context of the present invention, the term “immunologically equivalent” can be used with respect to the immunological effects or properties of a peptide used for immunization. For example, an amino acid sequence is immunologically equivalent to a reference amino acid sequence if said amino acid sequence when exposed to the immune system of a subject induces an immune reaction having a specificity of reacting with the reference amino acid sequence.

The term “immune effector functions” in the context of the present invention is encompassed by the term “immunological reaction” as used herein and includes any functions mediated by components of the immune system that result, for example, in the killing of tumor cells, or in the inhibition of tumor growth and/or inhibition of tumor development, including inhibition of tumor dissemination and metastasis. Preferably, the immune effector functions in the context of the present invention are T cell mediated effector functions. Such functions comprise in the case of a helper T cell (CD4+ T cell) the recognition of an antigen or an antigen peptide derived from an antigen in the context of MHC class II molecules by T cell receptors, the release of cytokines and/or the activation of CD8+ lymphocytes (CTLs) and/or B-cells, and in the case of CTL the recognition of an antigen or an antigen peptide derived from an antigen in the context of MHC class I molecules by T cell receptors, the elimination of cells presented in the context of MHC class I molecules, i.e., cells characterized by presentation of an antigen with class I MHC, for example, via apoptosis or perform-mediated cell lysis, production of cytokines such as IFN-γ and TNF-α, and specific cytolytic killing of antigen expressing target cells.

The terms “major histocompatibility complex” and the abbreviation “MHC” include MHC class I and MHC class II molecules and relate to a complex of genes which occurs in all vertebrates. MHC proteins or molecules are important for signaling between lymphocytes and antigen presenting cells or diseased cells in immune reactions, wherein the MHC proteins or molecules bind peptides and present them for recognition by T cell receptors. The proteins encoded by the MHC are expressed on the surface of cells, and display both self antigens (peptide fragments from the cell itself) and non-self antigens (e.g., fragments of invading microorganisms) to a T cell.

The MHC region is divided into three subgroups, class I, class II, and class III. MHC class I proteins contain an α-chain and β2-microglobulin (not part of the MHC encoded by chromosome 15). They present antigen fragments to cytotoxic T cells. On most immune system cells, specifically on antigen-presenting cells, MHC class II proteins contain α- and β-chains and they present antigen fragments to T-helper cells. MHC class III region encodes for other immune components, such as complement components and some that encode cytokines.

The MHC is both polygenic (there are several MHC class I and MHC class II genes) and polymorphic (there are multiple alleles of each gene).

As used herein, the term “haplotype” refers to the HLA alleles found on one chromosome and the proteins encoded thereby. Haplotype may also refer to the allele present at any one locus within the MHC. Each class of MHC is represented by several loci: e.g., HLA-A (Human Leukocyte Antigen-A), HLA-B, HLA-C, HLA-E, HLA-F, HLA-G, HLA-H, HLA-J, HLA-K, HLA-L, HLA-P and HLA-V for class I and HLA-DRA, HLA-DRB1-9, HLA-DQA1, HLA-DQB1, HLA-DPA1, HLA-DPB1, HLA-DMA, HLA-DMB, HLA-DOA, and HLA-DOB for class II. The terms “HLA allele” and “MHC allele” are used interchangeably herein.

The MHCs exhibit extreme polymorphism. Within the human population there are, at each genetic locus, a great number of haplotypes comprising distinct alleles. Different polymorphic MHC alleles, of both class I and class II, have different peptide specificities in that each allele encodes proteins that bind peptides exhibiting particular sequence patterns.

In the context of the present invention, an MHC molecule is preferably an HLA molecule.

In the context of the present invention, the term “MHC binding peptide” includes MHC class I and/or class II binding peptides or peptides that can be processed to produce MHC class I and/or class II binding peptides. In the case of class I MHC/peptide complexes, the binding peptides are typically 8-12, preferably 8-10 amino acids long although longer or shorter peptides may be effective. In the case of class II MHC/peptide complexes, the binding peptides are typically 9-30, preferably 10-25 amino acids long and are in particular 13-18 amino acids long, whereas longer and shorter peptides may be effective.

In an embodiment, an immunotherapeutic agent useful in the methods of the invention can comprise an antigen peptide or a nucleic acid encoding the antigen peptide. An “antigen peptide” preferably relates to a portion or fragment of an antigen which is capable of stimulating an immune response, preferably a cellular response against the antigen or cells characterized by expression of the antigen and preferably by presentation of the antigen such as diseased cells, in particular cancer cells. Preferably, an antigen peptide is capable of stimulating a cellular response against a cell characterized by presentation of an antigen with class I MHC and preferably is capable of stimulating an antigen-responsive cytotoxic T-lymphocyte (CTL). Preferably, the antigen peptides are MHC class I and/or class II presented peptides or can be processed to produce MHC class I and/or class II presented peptides. Preferably, the antigen peptides comprise an amino acid sequence substantially corresponding to the amino acid sequence of a fragment of an antigen. Preferably, said fragment of an antigen is an MHC class I and/or class II presented peptide. Preferably, an antigen peptide comprises an amino acid sequence substantially corresponding to the amino acid sequence of such fragment and is processed to produce such fragment, i.e., an MHC class I and/or class II presented peptide derived from an antigen.

If a peptide is to be presented directly, i.e., without processing, in particular without cleavage, it has a length which is suitable for binding to an MHC molecule, in particular a class I MHC molecule, and preferably is 7-20 amino acids in length, more preferably 7-12 amino acids in length, more preferably 8-11 amino acids in length, in particular 9 or 10 amino acids in length.

If a peptide is part of a larger entity comprising additional sequences, e.g. of a vaccine sequence or polypeptide, and is to be presented following processing, in particular following cleavage, the peptide produced by processing has a length which is suitable for binding to an MHC molecule, in particular a class I MHC molecule, and preferably is 7-20 amino acids in length, more preferably 7-12 amino acids in length, more preferably 8-11 amino acids in length, in particular 9 or 10 amino acids in length. Preferably, the sequence of the peptide which is to be presented following processing is derived from the amino acid sequence of an antigen, i.e., its sequence substantially corresponds and is preferably completely identical to a fragment of an antigen. Thus, an MHC binding peptide comprises a sequence which substantially corresponds and is preferably completely identical to a fragment of an antigen.

Peptides having amino acid sequences substantially corresponding to a sequence of a peptide which is presented by the class I MHC may differ at one or more residues that are not essential for TCR recognition of the peptide as presented by the class I MHC, or for peptide binding to MHC. Such substantially corresponding peptides are also capable of stimulating an antigen-responsive CTL and may be considered immunologically equivalent. Peptides having amino acid sequences differing from a presented peptide at residues that do not affect TCR recognition but improve the stability of binding to MHC may improve the immunogenicity of the antigen peptide, and may be referred to herein as “optimized peptide”. Using existing knowledge about which of these residues may be more likely to affect binding either to the MHC or to the TCR, a rational approach to the design of substantially corresponding peptides may be employed. Resulting peptides that are functional are contemplated as antigen peptides.

An antigen peptide when presented by MHC should be recognizable by a T cell receptor. Preferably, the antigen peptide if recognized by a T cell receptor is able to induce in the presence of appropriate co-stimulatory signals, clonal expansion of the T cell carrying the T cell receptor specifically recognizing the antigen peptide. Preferably, antigen peptides, in particular if presented in the context of MHC molecules, are capable of stimulating an immune response, preferably a cellular response against the antigen from which they are derived or cells characterized by expression of the antigen and preferably characterized by presentation of the antigen. Preferably, an antigen peptide is capable of stimulating a cellular response against a cell characterized by presentation of the antigen with class I MHC and preferably is capable of stimulating an antigen-responsive CTL. Such cell preferably is a target cell.

The term “genome” relates to the total amount of genetic information in the chromosomes of an organism or a cell.

The term “exome” refers to part of the genome of an organism formed by exons, which are coding portions of expressed genes. The exome provides the genetic blueprint used in the synthesis of proteins and other functional gene products. It is the most functionally relevant part of the genome and, therefore, it is most likely to contribute to the phenotype of an organism. The exome of the human genome is estimated to comprise 1.5% of the total genome (Ng et al., 2008, PLoS Gen., 4(8):1-15).

The term “transcriptome” relates to the set of all RNA molecules, including mRNA, rRNA, tRNA, and other non-coding RNA produced in one cell or a population of cells. In context of the present invention the transcriptome means the set of all RNA molecules produced in one cell, a population of cells, preferably a population of cancer cells, or all cells of a given individual at a certain time point.

A “nucleic acid” is preferably deoxyribonucleic acid (DNA) or ribonucleic acid (RNA), more preferably RNA, most preferably in vitro transcribed RNA (IVT RNA) or synthetic RNA. Nucleic acids include genomic DNA, cDNA, mRNA, recombinantly produced and chemically synthesized molecules. A nucleic acid may be present as a single-stranded or double-stranded and linear or covalently circularly closed molecule. A nucleic acid can be isolated. The term “isolated nucleic acid” means that the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic can be employed for introduction into, i.e. transfection of, cells, in particular, in the form of RNA which can be prepared by in vitro transcription from a DNA template. The RNA can moreover be modified before application by stabilizing sequences, capping, and polyadenylation.

The term “genetic material” refers to isolated nucleic acid, either DNA or RNA, a section of a double helix, a section of a chromosome, or an organism's or a cell's entire genome, in particular its exome or transcriptome.

The term “mutation” refers to a change of or difference in the nucleic acid sequence (nucleotide substitution, addition or deletion) compared to a reference. A “somatic mutation” can occur in any of the cells of the body except the germ cells (sperm and egg) and therefore are not passed on to children. These alterations can (but do not always) cause cancer or other diseases. Preferably a mutation is a non-synonymous mutation. The term “non-synonymous mutation” refers to a mutation, preferably a nucleotide substitution, which results in an amino acid change such as an amino acid substitution in the translation product, which preferably results in the formation of a neoepitope.

The term “mutation” includes point mutations, indels, fusions, chromothripsis and RNA edits.

The term “indel” describes a special mutation class, defined as a mutation resulting in a colocalized insertion and deletion and a net gain or loss in nucleotides. In coding regions of the genome, unless the length of an indel is a multiple of 3, they produce a frameshift mutation. Indels can be contrasted with a point mutation; where an indel inserts and deletes nucleotides from a sequence, a point mutation is a form of substitution that replaces one of the nucleotides.

Fusions can generate hybrid genes formed from two previously separate genes. It can occur as the result of a translocation, interstitial deletion, or chromosomal inversion. Often, fusion genes are oncogenes. Oncogenic fusion genes may lead to a gene product with a new or different function from the two fusion partners. Alternatively, a proto-oncogene is fused to a strong promoter, and thereby the oncogenic function is set to function by an upregulation caused by the strong promoter of the upstream fusion partner. Oncogenic fusion transcripts may also be caused by trans-splicing or read-through events.

The term “chromothripsis” refers to a genetic phenomenon by which specific regions of the genome are shattered and then stitched together via a single devastating event.

The term “RNA edit” or “RNA editing” refers to molecular processes in which the information content in an RNA molecule is altered through a chemical change in the base makeup. RNA editing includes nucleoside modifications such as cytidine (C) to uridine (U) and adenosine (A) to inosine (I) deaminations, as well as non-templated nucleotide additions and insertions. RNA editing in mRNAs effectively alters the amino acid sequence of the encoded protein so that it differs from that predicted by the genomic DNA sequence.

The term “cancer mutation signature” refers to a set of mutations which are present in cancer cells when compared to non-cancerous reference cells.

A “reference” in the context of the present invention may be used to correlate and compare the results obtained from a tumor specimen. Typically the “reference” may be obtained on the basis of one or more normal specimens, in particular specimens which are not affected by a cancer disease, either obtained from a patient or one or more different individuals, preferably healthy individuals, in particular individuals of the same species. A “reference” can be determined empirically by testing a sufficiently large number of normal specimens.

Disease-specific mutations can be determined by any suitable sequencing method, Next Generation Sequencing (NGS) technologies being preferred. Third Generation Sequencing methods might substitute for the NGS technology in the future to speed up the sequencing step of the method. For clarification purposes: the terms “Next Generation Sequencing” or “NGS” in the context of the present invention mean all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks, preferably within 1-7 days or most preferably within less than 24 hours and allow, in principle, single cell sequencing approaches. Multiple NGS platforms which are commercially available or which are mentioned in the literature can be used in the context of the present invention e.g. those described in detail in Zhang et al., 2011, The impact of next-generation sequencing on genomics, J. Genet Genomics 38(3):95-109; or in Voelkerding et al., 2009, Next generation sequencing: From basic research to diagnostics, Clinical chemistry 55:641-658.

Preferably, DNA and RNA preparations serve as starting material for NGS. Such nucleic acids can be easily obtained from samples such as biological material, e.g. from fresh, flash-frozen or formalin-fixed paraffin embedded tumor tissues (FFPE) or from freshly isolated cells or from CTCs which are present in the peripheral blood of patients. Normal non-mutated genomic DNA or RNA can be extracted from normal, somatic tissue, however germline cells are preferred in the context of the present invention. Germline DNA or RNA is extracted from peripheral blood mononuclear cells (PBMCs) in patients with non-hematological malignancies. Although nucleic acids extracted from FFPE tissues or freshly isolated single cells are highly fragmented, they are suitable for NGS applications.

Several targeted NGS methods for exome sequencing are described in the literature (for review see, e.g., Teer and Mullikin, 2010, Human Mol Genet 19(2):R145-51), all of which can be used in conjunction with the present invention. Many of these methods (described e.g. as genome capture, genome partitioning, genome enrichment etc.) use hybridization techniques and include array-based (e.g., Hodges et al., 2007, Nat. Genet. 39:1522-1527) and liquid-based (e.g., Choi et al., 2009, Proc. Natl. Acad. Sci USA 106:19096-19101) hybridization approaches. Commercial kits for DNA sample preparation and subsequent exome capture are also available: for example, Illumina Inc. (San Diego, Calif.) offers the TruSeq™ DNA Sample Preparation Kit and the Exome Enrichment Kit TruSeq™ Exome Enrichment Kit.

In order to reduce the number of false positive findings in detecting cancer specific somatic mutations or sequence differences when comparing e.g. the sequence of a tumor sample to the sequence of a reference sample such as the sequence of a germ line sample it is preferred to determine the sequence in replicates of one or both of these sample types. Thus, it is preferred that the sequence of a reference sample such as the sequence of a germ line sample is determined twice, three times or more. Alternatively or additionally, the sequence of a tumor sample is determined twice, three times or more. It may also be possible to determine the sequence of a reference sample such as the sequence of a germ line sample and/or the sequence of a tumor sample more than once by determining at least once the sequence in genomic DNA and determining at least once the sequence in RNA of said reference sample and/or of said tumor sample. For example, by determining the variations between replicates of a reference sample such as a germ line sample the expected rate of false positive (FDR) somatic mutations as a statistical quantity can be estimated. Technical repeats of a sample should generate identical results and any detected mutation in this “same vs. same comparison” is a false positive. In particular, to determine the false discovery rate for somatic mutation detection in a tumor sample relative to a reference sample, a technical repeat of the reference sample can be used as a reference to estimate the number of false positives. Furthermore, various quality related metrics (e.g. coverage or SNP quality) may be combined into a single quality score using a machine learning approach. For a given somatic variation all other variations with an exceeding quality score may be counted, which enables a ranking of all variations in a dataset.

In the context of the present invention, the term “RNA” relates to a molecule which comprises at least one ribonucleotide residue and preferably being entirely or substantially composed of ribonucleotide residues. “Ribonucleotide” relates to a nucleotide with a hydroxyl group at the 2′-position of a β-D-ribofuranosyl group. The term “RNA” comprises double-stranded RNA, single-stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

The term “RNA” includes and preferably relates to “mRNA”. The term “mRNA” means “messenger-RNA” and relates to a “transcript” which may be generated by using a DNA template and encodes a peptide or polypeptide. Typically, an mRNA comprises a 5 ‘-UTR, a protein coding region, and a 3’-UTR. mRNA only possesses limited half-life in cells and in vitro. In the context of the present invention, mRNA may be generated by in vitro transcription from a DNA template. The in vitro transcription methodology is known to the skilled person. For example, there is a variety of in vitro transcription kits commercially available.

The stability and translation efficiency of RNA may be modified as required. For example, RNA may be stabilized and its translation increased by one or more modifications having a stabilizing effects and/or increasing translation efficiency of RNA. Such modifications are described, for example, in PCT/EP2006/009448 incorporated herein by reference. In order to increase expression of the RNA used in embodiments of the present invention, it may be modified within the coding region, i.e. the sequence encoding the expressed peptide or protein, preferably without altering the sequence of the expressed peptide or protein, so as to increase the GC-content to increase mRNA stability and to perform a codon optimization and, thus, enhance translation in cells.

The term “modification” in the context of the RNA used in the present invention includes any modification of an RNA which is not naturally present in said RNA.

In one embodiment of the invention, the RNA used according to the invention does not have uncapped 5′-triphosphates. Removal of such uncapped 5′-triphosphates can be achieved by treating RNA with a phosphatase.

The RNA according to the invention may have modified ribonucleotides in order to increase its stability and/or decrease cytotoxicity. For example, in one embodiment, in the RNA used according to the invention 5-methylcytidine is substituted partially or completely, preferably completely, for cytidine. Alternatively or additionally, in one embodiment, in the RNA used according to the invention pseudouridine is substituted partially or completely for uridine. In a preferred embodiment, the RNA according to the invention having modified ribonucleotides still has the ability to act as a Toll-like receptor agonist.

In one embodiment, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap (m⁷G). In the context of the present invention, the term “5′-cap” includes a 5′-cap analog that resembles the RNA cap structure and is modified to possess the ability to stabilize RNA and/or enhance translation of RNA if attached thereto, preferably in vivo and/or in a cell.

Providing an RNA with a 5′-cap or 5′-cap analog may be achieved by in vitro transcription of a DNA template in presence of said 5′-cap or 5′-cap analog, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

The RNA may comprise further modifications. For example, a further modification of the RNA used in the present invention may be an extension or truncation of the naturally occurring poly(A) tail or an alteration of the 5′- or 3′-untranslated regions (UTR) such as introduction of a UTR which is not related to the coding region of said RNA, for example, the exchange of the existing 3′-UTR with or the insertion of one or more, preferably two copies of a 3′-UTR derived from a globin gene, such as alpha2-globin, alpha1-globin, beta-globin, preferably beta-globin, more preferably human beta-globin.

RNA having an unmasked poly-A sequence is translated more efficiently than RNA having a masked poly-A sequence. The term “poly(A) tail” or “poly-A sequence” relates to a sequence of adenyl (A) residues which typically is located on the 3′-end of a RNA molecule and “unmasked poly-A sequence” means that the poly-A sequence at the 3′ end of an RNA molecule ends with an A of the poly-A sequence and is not followed by nucleotides other than A located at the 3′ end, i.e. downstream, of the poly-A sequence. Furthermore, a long poly-A sequence of about 120 base pairs results in an optimal transcript stability and translation efficiency of RNA.

Therefore, in order to increase stability and/or expression of the RNA used according to the present invention, it may be modified so as to be present in conjunction with a poly-A sequence, preferably having a length of 10 to 500, more preferably 30 to 300, even more preferably 65 to 200 and especially 100 to 150 adenosine residues. In an especially preferred embodiment the poly-A sequence has a length of approximately 120 adenosine residues. To further increase stability and/or expression of the RNA used according to the invention, the poly-A sequence can be unmasked.

In addition, incorporation of a 3′-non translated region (UTR) into the 3′-non translated region of an RNA molecule can result in an enhancement in translation efficiency. A synergistic effect may be achieved by incorporating two or more of such 3′-non translated regions. The 3′-non translated regions may be autologous or heterologous to the RNA into which they are introduced. In one particular embodiment the 3′-non translated region is derived from the human β-globin gene.

A combination of the above described modifications, i.e. incorporation of a poly-A sequence, unmasking of a poly-A sequence and incorporation of one or more 3′-non translated regions, has a synergistic influence on the stability of RNA and increase in translation efficiency.

The term “stability” of RNA relates to the “half-life” of RNA. “Half-life” relates to the period of time which is needed to eliminate half of the activity, amount, or number of molecules. In the context of the present invention, the half-life of an RNA is indicative for the stability of said RNA. The half-life of RNA may influence the “duration of expression” of the RNA. It can be expected that RNA having a long half-life will be expressed for an extended time period.

Of course, if it is desired to decrease stability and/or translation efficiency of RNA, it is possible to modify RNA so as to interfere with the function of elements as described above increasing the stability and/or translation efficiency of RNA.

The term “expression” is used in its most general meaning and comprises the production of RNA and/or peptides or polypeptides, e.g. by transcription and/or translation. With respect to RNA, the term “expression” or “translation” relates in particular to the production of peptides or polypeptides. It also comprises partial expression of nucleic acids. Moreover, expression can be transient or stable.

The term expression also includes an “aberrant expression” or “abnormal expression”. “Aberrant expression” or “abnormal expression” means that expression is altered, preferably increased, compared to a reference, e.g. a state in a subject not having a disease associated with aberrant or abnormal expression of a certain protein, e.g., a tumor antigen. An increase in expression refers to an increase by at least 10%, in particular at least 20%, at least 50% or at least 100%, or more. In one embodiment, expression is only found in a diseased tissue, while expression in a healthy tissue is repressed.

The term “specifically expressed” means that a protein is essentially only expressed in a specific tissue or organ. For example, a tumor antigen specifically expressed in gastric mucosa means that said protein is primarily expressed in gastric mucosa and is not expressed in other tissues or is not expressed to a significant extent in other tissue or organ types. Thus, a protein that is exclusively expressed in cells of the gastric mucosa and to a significantly lesser extent in any other tissue, such as testis, is specifically expressed in cells of the gastric mucosa. In some embodiments, a tumor antigen may also be specifically expressed under normal conditions in more than one tissue type or organ, such as in 2 or 3 tissue types or organs, but preferably in not more than 3 different tissue or organ types. In this case, the tumor antigen is then specifically expressed in these organs. For example, if a tumor antigen is expressed under normal conditions preferably to an approximately equal extent in lung and stomach, said tumor antigen is specifically expressed in lung and stomach.

In the context of the present invention, the term “transcription” relates to a process, wherein the genetic code in a DNA sequence is transcribed into RNA. Subsequently, the RNA may be translated into protein. According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are encompassed by the term “vector”. The RNA used in the present invention preferably is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

The term “translation” relates to the process in the ribosomes of a cell by which a strand of messenger RNA directs the assembly of a sequence of amino acids to make a peptide or polypeptide.

Expression control sequences or regulatory sequences, which in the context of the present invention may be linked functionally with a nucleic acid, can be homologous or heterologous with respect to the nucleic acid. A coding sequence and a regulatory sequence are linked together “functionally” if they are bound together covalently, so that the transcription or translation of the coding sequence is under the control or under the influence of the regulatory sequence. If the coding sequence is to be translated into a functional protein, with functional linkage of a regulatory sequence with the coding sequence, induction of the regulatory sequence leads to a transcription of the coding sequence, without causing a reading frame shift in the coding sequence or inability of the coding sequence to be translated into the desired protein or peptide.

The term “expression control sequence” or “regulatory sequence” comprises, in the context of the invention, promoters, ribosome-binding sequences and other control elements, which control the transcription of a nucleic acid or the translation of the derived RNA. In an embodiment, the regulatory sequences can be controlled. The precise structure of regulatory sequences can vary depending on the species or depending on the cell type, but generally comprises 5′-untranscribed and 5′- and 3′-untranslated sequences, which are involved in the initiation of transcription or translation, such as TATA-box, capping-sequence, CAAT-sequence and the like. In particular, 5′-untranscribed regulatory sequences comprise a promoter region that includes a promoter sequence for transcriptional control of the functionally bound gene. Regulatory sequences can also comprise enhancer sequences or upstream activator sequences.

Preferably, the RNA to be expressed in a cell is introduced into said cell. In one embodiment of the methods according to the invention, the RNA that is to be introduced into a cell is obtained by in vitro transcription of an appropriate DNA template.

Terms such as “RNA capable of expressing” and “RNA encoding” are used interchangeably herein and with respect to a particular peptide or polypeptide mean that the RNA, if present in the appropriate environment, preferably within a cell, can be expressed to produce said peptide or polypeptide. Preferably, RNA is able to interact with the cellular translation machinery to provide the peptide or polypeptide it is capable of expressing.

Terms such as “transferring”, “introducing” or “transfecting” are used interchangeably herein and relate to the introduction of nucleic acids, in particular exogenous or heterologous nucleic acids, in particular RNA into a cell. According to the present invention, the cell can form part of an organ, a tissue and/or an organism. According to the present invention, the administration of a nucleic acid is either achieved as naked nucleic acid or in combination with an administration reagent. Preferably, administration of nucleic acids is in the form of naked nucleic acids. Preferably, the RNA is administered in combination with stabilizing substances such as RNase inhibitors. The present invention also envisions the repeated introduction of nucleic acids into cells to allow sustained expression for extended time periods.

Cells can be transfected with any carriers with which RNA can be associated, e.g. by forming complexes with the RNA or forming vesicles in which the RNA is enclosed or encapsulated, resulting in increased stability of the RNA compared to naked RNA. Useful carriers include, for example, lipid-containing carriers such as cationic lipids, liposomes, in particular cationic liposomes, and micelles, and nanoparticles. Cationic lipids may form complexes with negatively charged nucleic acids. Any cationic lipid may be used.

Preferably, the introduction of RNA which encodes a peptide or polypeptide into a cell, in particular into a cell present in vivo, results in expression of said peptide or polypeptide in the cell. In particular embodiments, the targeting of the nucleic acids to particular cells is preferred. In such embodiments, a carrier which is applied for the administration of the nucleic acid to a cell (for example, a retrovirus or a liposome), exhibits a targeting molecule. For example, a molecule such as an antibody which is specific for a surface membrane protein on the target cell or a ligand for a receptor on the target cell may be incorporated into the nucleic acid carrier or may be bound thereto. In case the nucleic acid is administered by liposomes, proteins which bind to a surface membrane protein which is associated with endocytosis may be incorporated into the liposome formulation in order to enable targeting and/or uptake. Such proteins encompass capsid proteins of fragments thereof which are specific for a particular cell type, antibodies against proteins which are internalized, proteins which target an intracellular location, etc.

The term “cell” or “host cell” preferably is an intact cell, i.e. a cell with an intact membrane that has not released its normal intracellular components such as enzymes, organelles, or genetic material. An intact cell preferably is a viable cell, i.e. a living cell capable of carrying out its normal metabolic functions. Preferably said term relates to any cell which can be transformed or transfected with an exogenous nucleic acid. The term “cell” includes prokaryotic cells (e.g., E. coli) or eukaryotic cells (e.g., dendritic cells, B cells, CHO cells, COS cells, K562 cells, HEK293 cells, HELA cells, yeast cells, and insect cells). The exogenous nucleic acid may be found inside the cell (i) freely dispersed as such, (ii) incorporated in a recombinant vector, or (iii) integrated into the host cell genome or mitochondrial DNA. Mammalian cells are particularly preferred, such as cells from humans, mice, hamsters, pigs, goats, and primates. The cells may be derived from a large number of tissue types and include primary cells and cell lines. Specific examples include keratinocytes, peripheral blood leukocytes, bone marrow stem cells, and embryonic stem cells. In further embodiments, the cell is an antigen-presenting cell, in particular a dendritic cell, a monocyte, or macrophage.

A cell which comprises a nucleic acid molecule preferably expresses the peptide or polypeptide encoded by the nucleic acid.

The term “clonal expansion” refers to a process wherein a specific entity is multiplied. In the context of the present invention, the term is preferably used in the context of an immunological response in which lymphocytes are stimulated by an antigen, proliferate, and the specific lymphocyte recognizing said antigen is amplified. Preferably, clonal expansion leads to differentiation of the lymphocytes.

Terms such as “reducing” or “inhibiting” relate to the ability to cause an overall decrease, preferably of 5% or greater, 10% or greater, 20% or greater, more preferably of 50% or greater, and most preferably of 75% or greater, in the level. The term “inhibit” or similar phrases includes a complete or essentially complete inhibition, i.e. a reduction to zero or essentially to zero.

Terms such as “increasing”, “enhancing”, “promoting” or “prolonging” preferably relate to an increase, enhancement, promotion or prolongation by about at least 10%, preferably at least 20%, preferably at least 30%, preferably at least 40%, preferably at least 50%, preferably at least 80%, preferably at least 100%, preferably at least 200% and in particular at least 300%. These terms may also relate to an increase, enhancement, promotion or prolongation from zero or a non-measurable or non-detectable level to a level of more than zero or a level which is measurable or detectable.

According to the present invention, the term “peptide” refers to substances comprising two or more, preferably 3 or more, preferably 4 or more, preferably 6 or more, preferably 8 or more, preferably 10 or more, preferably 13 or more, preferably 16 more, preferably 21 or more and up to preferably 8, 10, 20, 30, 40 or 50, in particular 100 amino acids joined covalently by peptide bonds. The term “polypeptide” or “protein” refers to large peptides, preferably to peptides with more than 100 amino acid residues, but in general the terms “peptide”, “polypeptide” and “protein” are synonyms and are used interchangeably herein. According to the invention, the term “modification” or “sequence change” with respect to peptides, polypeptides or proteins relates to a sequence change in a peptide, polypeptide or protein compared to a parental sequence such as the sequence of a wildtype peptide, polypeptide or protein. The term includes amino acid insertion variants, amino acid addition variants, amino acid deletion variants and amino acid substitution variants, preferably amino acid substitution variants. All these sequence changes according to the invention may potentially create new epitopes.

Amino acid insertion variants comprise insertions of single or two or more amino acids in a particular amino acid sequence.

Amino acid addition variants comprise amino- and/or carboxy-terminal fusions of one or more amino acids, such as 1, 2, 3, 4 or 5, or more amino acids.

Amino acid deletion variants are characterized by the removal of one or more amino acids from the sequence, such as by removal of 1, 2, 3, 4 or 5, or more amino acids.

Amino acid substitution variants are characterized by at least one residue in the sequence being removed and another residue being inserted in its place.

According to the invention, a modification or modified peptide used for testing in the methods of the invention may be derived from a protein comprising a modification.

The term “derived” means according to the invention that a particular entity, in particular a particular peptide sequence, is present in the object from which it is derived. In the case of amino acid sequences, especially particular sequence regions, “derived” in particular means that the relevant amino acid sequence is derived from an amino acid sequence in which it is present.

Immunotherapeutic agents, once a suitable dose has been determined by the methods described herein, can be used to treat a subject with a disease, e.g., a disease characterized by the presence of diseased cells expressing an antigen and presenting an antigen peptide, by administering the immunotherapeutic agent at the suitable dose. Particularly preferred diseases are cancer diseases. The immunotherapeutic agents described herein may also be used for immunization or vaccination to prevent a disease described herein.

One such immunotherapeutic agent is a vaccine such as a cancer vaccine designed on the basis of neoepitopes that are expressed only in cancer cells.

According to the invention, the term “vaccine” relates to a pharmaceutical preparation (pharmaceutical composition) or product that upon administration induces an immune response, in particular a cellular immune response, which recognizes and attacks a pathogen or a diseased cell such as a cancer cell. A vaccine may be used for the prevention or treatment of a disease. The term “personalized cancer vaccine” or “individualized cancer vaccine” concerns a particular cancer patient and means that a cancer vaccine is adapted to the needs or special circumstances of an individual cancer patient.

The cancer vaccines provided according to the invention when administered to a patient may provide one or more T cell epitopes for stimulating, priming and/or expanding T cells specific for the patient's tumor. The T cells are preferably directed against cells expressing antigens from which the T cell epitopes are derived. Thus, the vaccines described herein are preferably capable of inducing or promoting a cellular response, preferably cytotoxic T cell activity, against a cancer disease characterized by presentation of one or more tumor-associated neoantigens with class I MHC. Since a vaccine provided herein will target cancer specific mutations it will be specific for the patient's tumor.

In one embodiment of the present invention, a vaccine relates to a vaccine which when administered to a patient preferably provides one or more T cell epitopes (neoepitopes, suitable neoepitopes, combination of suitable neoepitopes identified herein), such as 2 or more, 5 or more, 10 or more, 15 or more, 20 or more, 25 or more, 30 or more and preferably up to 60, up to 55, up to 50, up to 45, up to 40, up to 35 or up to 30 T cell epitopes, incorporating amino acid modifications or modified peptides predicted as being suitable epitopes. Presentation of these epitopes by cells of a patient, in particular antigen presenting cells, preferably results in T cells targeting the epitopes when bound to MHC and thus, the patient's tumor, preferably the primary tumor as well as tumor metastases, expressing antigens from which the T cell epitopes are derived and presenting the same epitopes on the surface of the tumor cells.

Further steps may be taken to determine the usability of the identified amino acid modifications or modified peptides containing an epitope for cancer vaccination. Thus further steps can involve one or more of the following: (i) assessing whether the modifications are located in known or predicted MHC presented epitopes, (ii) in vitro and/or in silico testing whether the modifications are located in MHC presented epitopes, e.g. testing whether the modifications are part of peptide sequences which are processed into and/or presented as MHC presented epitopes, and (iii) in vitro testing whether the envisaged modified epitopes, in particular when present in their natural sequence context, e.g. when flanked by amino acid sequences also flanking said epitopes in the naturally occurring protein, and when expressed in antigen presenting cells are able to stimulate T cells such as T cells of the patient having the desired specificity. Such flanking sequences each may comprise 3 or more, 5 or more, 10 or more, 15 or more, 20 or more and preferably up to 50, up to 45, up to 40, up to 35 or up to 30 amino acids and may flank the epitope sequence N-terminally and/or C-terminally.

Modified peptides determined according to the invention may be ranked for their usability as epitopes for cancer vaccination. Thus, in one aspect, a manual or computer-based analytical process may be used in which the identified modified peptides are analyzed and selected for their usability in the respective vaccine to be provided. In a preferred embodiment, said analytical process is a computational algorithm-based process. Preferably, said analytical process comprises determining and/or ranking epitopes according to a prediction of their capacity of being immunogenic.

The epitopes identified according to the invention and provided in a vaccine are preferably present in the faun of a polypeptide comprising said epitopes such as a polyepitopic polypeptide or a nucleic acid, in particular RNA, encoding said polypeptide. Furthermore, the epitopes may be present in the polypeptide in the form of a vaccine sequence, i.e. present in their natural sequence context, e.g. flanked by amino acid sequences also flanking said epitopes in the naturally occurring protein. Such flanking sequences each may comprise 5 or more, 10 or more, 15 or more, 20 or more and preferably up to 50, up to 45, up to 40, up to 35 or up to 30 amino acids and may flank the epitope sequence N-terminally and/or C-terminally. Thus, a vaccine sequence may comprise 20 or more, 25 or more, 30 or more, 35 or more, 40 or more and preferably up to 50, up to 45, up to 40, up to 35 or up to 30 amino acids. In one embodiment, the epitopes and/or vaccine sequences are lined up in the polypeptide head-to-tail.

In one embodiment, the epitopes identified herein and/or vaccine sequences are spaced by linkers, in particular neutral linkers. The term “linker” used in the context of the present invention relates to a peptide added between two peptide domains such as epitopes or vaccine sequences to connect said peptide domains. There is no particular limitation regarding the linker sequence. However, it is preferred that the linker sequence reduces steric hindrance between the two peptide domains, is well translated, and supports or allows processing of the epitopes. Furthermore, the linker should have no or only little immunogenic sequence elements. Linkers preferably should not create non-endogenous epitopes like those generated from the junction suture between adjacent epitopes, which might generate unwanted immune reactions. Therefore, the polyepitopic vaccine should preferably contain linker sequences which are able to reduce the number of unwanted MHC binding junction epitopes. Hoyt et al. (EMBO J. 25(8), 1720-9, 2006) and Zhang et al. (J. Biol. Chem., 279(10), 8635-41, 2004) have shown that glycine-rich sequences impair proteasomal processing and thus the use of glycine rich linker sequences act to minimize the number of linker-contained peptides that can be processed by the proteasome. Furthermore, glycine was observed to inhibit a strong binding in MHC binding groove positions (Abastado et al., 1993, J. Immunol. 151(7):3569-75). Schlessinger et al., 2005, Proteins 61(1):115-26 had found that amino acids glycine and serine included in an amino acid sequence result in a more flexible protein that is more efficiently translated and processed by the proteasome, enabling better access to the encoded epitopes. The linker each may comprise 3 or more, 6 or more, 9 or more, 10 or more, 15 or more, 20 or more and preferably up to 50, up to 45, up to 40, up to 35 or up to 30 amino acids. Preferably the linker is enriched in glycine and/or serine amino acids. Preferably, at least 50%, at least 60%, at least 70%, at least 80%, at least 90%, or at least 95% of the amino acids of the linker are glycine and/or serine. In one preferred embodiment, a linker is substantially composed of the amino acids glycine and serine. In one embodiment, the linker comprises the amino acid sequence (GGS)ₐ(GSS)_{b}(GGG)_{c}(SSG)_{d}(GSG)ₑ wherein a, b, c, d and e is independently a number selected from 0, 1, 2, 3, 4, 5, 6, 7, 8, 9, 10, 11, 12, 13, 14, 15, 16, 17, 18, 19, or 20 and wherein a+b+c+d+e are different from 0 and preferably are 2 or more, 3 or more, 4 or more or 5 or more. In one embodiment, the linker comprises a sequence as described herein including the linker sequences described in the examples such as the sequence GGSGGGGSG.

In one particularly preferred embodiment, a polypeptide incorporating one or more neoepitopes, such as a polyepitopic polypeptide, is an immunotherapeutic agent that can be administered to a patient in the form of a nucleic acid, preferably RNA such as in vitro transcribed or synthetic RNA, which nucleic acid may be expressed in cells of a patient, such as antigen presenting cells, to produce the polypeptide. Also envisioned is the administration of one or more multiepitopic polypeptides which for the purpose of the present invention are comprised by the term “polyepitopic polypeptide”, preferably in the form of a nucleic acid, preferably RNA such as in vitro transcribed or synthetic RNA, which may be expressed in cells of a patient such as antigen presenting cells to produce the one or more polypeptides. In the case of an administration of more than one multiepitopic polypeptide the suitable neoepitopes provided by the different multiepitopic polypeptides may be different or partially overlapping. Once present in cells of a patient such as antigen presenting cells the polypeptide according to the invention is processed to produce the suitable neoepitopes identified according to the invention. Administration of a vaccine provided according to the invention may provide MHC class II-presented epitopes that are capable of eliciting a CD4+ helper T cell response against cells expressing antigens from which the MHC presented epitopes are derived. Alternatively or additionally, administration of a vaccine provided according to the invention may provide MHC class I-presented neoepitopes that are capable of eliciting a CD8+ T cell response against cells expressing antigens from which the MHC presented neoepitopes are derived. Furthermore, administration of a vaccine provided according to the invention may provide one or more neoepitopes (including known neoepitopes and suitable neoepitopes identified according to the invention) as well as one or more epitopes not containing cancer specific somatic mutations but being expressed by cancer cells and preferably inducing an immune response against cancer cells, preferably a cancer specific immune response. In one embodiment, administration of a vaccine provided according to the invention provides neoepitopes that are MHC class II-presented epitopes and/or are capable of eliciting a CD4+ helper T cell response against cells expressing antigens from which the MHC presented epitopes are derived as well as epitopes not containing cancer-specific somatic mutations that are MHC class I-presented epitopes and/or are capable of eliciting a CD8+ T cell response against cells expressing antigens from which the MHC presented epitopes are derived. In one embodiment, the epitopes not containing cancer-specific somatic mutations are derived from a tumor antigen. In one embodiment, the neoepitopes and epitopes not containing cancer-specific somatic mutations have a synergistic effect in the treatment of cancer. Preferably, a vaccine provided according to the invention is useful for polyepitopic stimulation of cytotoxic and/or helper T cell responses.

The vaccine provided according to the invention may be a recombinant vaccine.

The term “recombinant” in the context of the present invention means “made through genetic engineering”. Preferably, a “recombinant entity” such as a recombinant polypeptide in the context of the present invention is not occurring naturally, and preferably is a result of a combination of entities such as amino acid or nucleic acid sequences which are not combined in nature. For example, a recombinant polypeptide in the context of the present invention may contain several amino acid sequences such as neo-epitopes or vaccine sequences derived from different proteins or different portions of the same protein fused together, e.g., by peptide bonds or appropriate linkers.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

According to the invention, the term “disease” refers to any pathological state, including cancer diseases, in particular those forms of cancer diseases described herein.

The term “normal” refers to the healthy state or the conditions in a healthy subject or tissue, i.e., non-pathological conditions, wherein “healthy” preferably means non-cancerous.

“Disease involving cells expressing an antigen” means that expression of the antigen in cells of a diseased tissue or organ is detected. Expression in cells of a diseased tissue or organ may be increased compared to the state in a healthy tissue or organ. An increase refers to an increase by at least 10%, in particular at least 20%, at least 50%, at least 100%, at least 200%, at least 500%, at least 1000%, at least 10000% or even more. In one embodiment, expression is only found in a diseased tissue, while expression in a healthy tissue is repressed. According to the invention, diseases involving or being associated with cells expressing an antigen include cancer diseases.

Cancer (medical term: malignant neoplasm) is a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, and do not invade or metastasize. Most cancers form a tumor but some, like leukemia, do not.

Malignant tumor is essentially synonymous with cancer. Malignancy, malignant neoplasm, and malignant tumor are essentially synonymous with cancer.

According to the invention, the term “tumor” or “tumor disease” refers to an abnormal growth of cells (called neoplastic cells, tumorigenous cells or tumor cells) preferably forming a swelling or lesion. By “tumor cell” is meant an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be either benign, pre-malignant or malignant.

A benign tumor is a tumor that lacks all three of the malignant properties of a cancer. Thus, by definition, a benign tumor does not grow in an unlimited, aggressive manner, does not invade surrounding tissues, and does not spread to non-adjacent tissues (metastasize).

Neoplasm is an abnormal mass of tissue as a result of neoplasia. Neoplasia (new growth in Greek) is the abnormal proliferation of cells. The growth of the cells exceeds, and is uncoordinated with that of the normal tissues around it. The growth persists in the same excessive manner even after cessation of the stimuli. It usually causes a lump or tumor. Neoplasms may be benign, pre-malignant or malignant.

“Growth of a tumor” or “tumor growth” in the context of the present invention relates to the tendency of a tumor to increase its size and/or to the tendency of tumor cells to proliferate.

For purposes of the present invention, the terms “cancer” and “cancer disease” are used interchangeably with the terms “tumor” and “tumor disease”.

Cancers are classified by the type of cell that resembles the tumor and, therefore, the tissue presumed to be the origin of the tumor. These are the histology and the location, respectively.

The term “cancer” according to the invention comprises leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer and the metastases thereof. Examples thereof are lung carcinomas, mamma carcinomas, prostate carcinomas, colon carcinomas, renal cell carcinomas, cervical carcinomas, or metastases of the cancer types or tumors described above. The term cancer according to the invention also comprises cancer metastases and relapse of cancer.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential. In one embodiment, the term “metastasis” according to the invention relates to “distant metastasis” which relates to a metastasis which is remote from the primary tumor and the regional lymph node system.

The cells of a secondary or metastatic tumor are like those in the original tumor. This means, for example, that, if ovarian cancer metastasizes to the liver, the secondary tumor is made up of abnormal ovarian cells, not of abnormal liver cells. The tumor in the liver is then called metastatic ovarian cancer, not liver cancer.

The term “circulating tumor cells” or “CTCs” relates to cells that have detached from a primary tumor or tumor metastases and circulate in the bloodstream. CTCs may constitute seeds for subsequent growth of additional tumors (metastasis) in different tissues. Circulating tumor cells are found in frequencies in the order of 1-10 CTC per mL of whole blood in patients with metastatic disease. Research methods have been developed to isolate CTC. Several research methods have been described in the art to isolate CTCs, e.g. techniques which use of the fact that epithelial cells commonly express the cell adhesion protein EpCAM, which is absent in normal blood cells. Immunomagnetic bead-based capture involves treating blood specimens with antibody to EpCAM that has been conjugated with magnetic particles, followed by separation of tagged cells in a magnetic field. Isolated cells are then stained with antibody to another epithelial marker, cytokeratin, as well as a common leukocyte marker CD45, so as to distinguish rare CTCs from contaminating white blood cells. This robust and semi-automated approach identifies CTCs with an average yield of approximately 1 CTC/mL and a purity of 0.1% (Allard et al., 2004, Clin Cancer Res 10:6897-6904). A second method for isolating CTCs uses a microfluidic-based CTC capture device which involves flowing whole blood through a chamber embedded with 80,000 microposts that have been rendered functional by coating with antibody to EpCAM. CTCs are then stained with secondary antibodies against either cytokeratin or tissue specific markers, such as PSA in prostate cancer or HER2 in breast cancer and are visualized by automated scanning of microposts in multiple planes along three dimensional coordinates. CTC-chips are able to identifying cytokerating-positive circulating tumor cells in patients with a median yield of 50 cells/ml and purity ranging from 1-80% (Nagrath et al., 2007, Nature 450:1235-1239). Another possibility for isolating CTCs is using the CellSearch™ Circulating Tumor Cell (CTC) Test from Veridex, LLC (Raritan, N.J.) which captures, identifies, and counts CTCs in a tube of blood. The CellSearch™ system is a U.S. Food and Drug Administration (FDA) approved methodology for enumeration of CTC in whole blood which is based on a combination of immunomagnetic labeling and automated digital microscopy. There are other methods for isolating CTCs described in the literature all of which can be used in conjunction with the present invention.

A relapse or recurrence occurs when a person is affected again by a condition that affected them in the past. For example, if a patient has suffered from a tumor disease, has received a successful treatment of said disease and again develops said disease said newly developed disease may be considered as relapse or recurrence. However, according to the invention, a relapse or recurrence of a tumor disease may but does not necessarily occur at the site of the original tumor disease. Thus, for example, if a patient has suffered from ovarian tumor and has received a successful treatment a relapse or recurrence may be the occurrence of an ovarian tumor or the occurrence of a tumor at a site different to ovary. A relapse or recurrence of a tumor also includes situations wherein a tumor occurs at a site different to the site of the original tumor as well as at the site of the original tumor. Preferably, the original tumor for which the patient has received a treatment is a primary tumor and the tumor at a site different to the site of the original tumor is a secondary or metastatic tumor.

The ten “immunotherapy” relates to the treatment of a disease or condition by inducing, enhancing, or suppressing an immune response. Immunotherapies designed to elicit or amplify an immune response are classified as activation immunotherapies, while immunotherapies that reduce or suppress an immune response are classified as suppression immunotherapies. The term “immunotherapy” includes antigen immunization or antigen vaccination, or tumor immunization or tumor vaccination. The term “immunotherapy” also relates to the manipulation of immune responses such that inappropriate immune responses are modulated into more appropriate ones in the context of autoimmune diseases such as rheumatic arthritis, allergies, diabetes or multiple sclerosis.

The terms “immunization” or “vaccination” describe the process of administering an antigen to an individual with the purpose of inducing an immune response, for example, for therapeutic or prophylactic reasons.

By “treat” is meant to administer an immunotherapeutic agent or composition comprising the immunotherapeutic agent as described herein to a subject in order to prevent or eliminate a disease, including reducing the size of a tumor or the number of tumors in a subject; arrest or slow a disease in a subject; inhibit or slow the development of a new disease in a subject; decrease the frequency or severity of symptoms and/or recurrences in a subject who currently has or who previously has had a disease; and/or prolong, i.e. increase the lifespan of the subject. In particular, the term “treatment of a disease” includes curing, shortening the duration, ameliorating, preventing, slowing down or inhibiting progression or worsening, or preventing or delaying the onset of a disease or the symptoms thereof.

By “being at risk” is meant a subject, i.e. a patient, that is identified as having a higher than normal chance of developing a disease, in particular cancer, compared to the general population. In addition, a subject who has had, or who currently has, a disease, in particular cancer, is a subject who has an increased risk for developing a disease, as such a subject may continue to develop a disease. Subjects who currently have, or who have had, a cancer also have an increased risk for cancer metastases.

A prophylactic administration of an immunotherapy, for example, a prophylactic administration of the immunotherapeutic agent or composition comprising the immunotherapeutic agent, preferably protects the recipient from the development of a disease. A therapeutic administration of an immunotherapy, for example, a therapeutic administration of the immunotherapeutic agent, may lead to the inhibition of the progress/growth of the disease. This comprises the deceleration of the progress/growth of the disease, in particular a disruption of the progression of the disease, which preferably leads to elimination of the disease.

Immunotherapy may be performed using any of a variety of techniques, in which agents provided herein function to remove diseased cells from a patient. Such removal may take place as a result of enhancing or inducing an immune response in a patient specific for an antigen or a cell expressing an antigen.

The immunotherapeutic agents and compositions may be used alone or in combination with conventional therapeutic regimens such as surgery, irradiation, chemotherapy and/or bone marrow transplantation (autologous, syngeneic, allogeneic or unrelated).

The term “in vivo” relates to the situation in a subject.

The terms “subject”, “individual”, “organism” or “patient” relate to vertebrates, particularly mammals and are used interchangeably herein. For example, mammals in the context of the present invention are humans, non-human primates, domesticated mammals such as dogs, cats, sheep, cattle, goats, pigs, horses etc., laboratory animals such as mice, rats, rabbits, guinea pigs, etc. as well as animals in captivity such as animals of zoos. The terms also relate to non-mammalian vertebrates such as birds (particularly domesticated birds such as chicken, ducks, geese, turkeys) and to fish (particularly farmed fish, e.g. salmon or catfish). The term “animal” as used herein also includes humans.

The term “autologous” is used to describe anything that is derived from the same subject. For example, “autologous transplant” refers to a transplant of tissue or organs derived from the same subject. Such procedures are advantageous because they overcome the immunological barrier which otherwise results in rejection.

The term “heterologous” is used to describe something consisting of multiple different elements. As an example, the transfer of one individual's bone marrow into a different individual constitutes a heterologous transplant. A heterologous gene is a gene derived from a source other than the subject.

As part of the composition for an immunization or a vaccination, preferably one or more immunotherapeutic agents are administered together with one or more adjuvants for inducing an immune response or for increasing an immune response. The term “adjuvant” relates to compounds which prolongs or enhances or accelerates an immune response. The composition of the present invention preferably exerts its effect without addition of adjuvants. Still, the composition of the present application may contain any known adjuvant. Adjuvants comprise a heterogeneous group of compounds such as oil emulsions (e.g., Freund's adjuvants), mineral compounds (such as alum), bacterial products (such as Bordetella pertussis toxin), liposomes, and immune-stimulating complexes. Examples for adjuvants are monophosphoryl-lipid-A (MPL SmithKline Beecham). Saponins such as QS21 (SmithKline Beecham), DQS21 (SmithKline Beecham; WO 96/33739), QS7, QS17, QS18, and QS-L1 (So et al., 1997, Mol. Cells 7: 178-186), incomplete Freund's adjuvants, complete Freund's adjuvants, vitamin E, montanid, alum, CpG oligonucleotides (Krieg et al., 1995, Nature 374: 546-549), and various water-in-oil emulsions which are prepared from biologically degradable oils such as squalene and/or tocopherol.

Other substances which stimulate an immune response of the patient may also be administered. It is possible, for example, to use cytokines in a vaccination, owing to their regulatory properties on lymphocytes. Such cytokines comprise, for example, interleukin-12 (IL-12) which was shown to increase the protective actions of vaccines (see, Hall, 1995, IL-12 at the crossroads, Science 268:1432-1434), GM-CSF and IL-18.

There are a number of compounds which enhance an immune response and which therefore may be used in a vaccination. Said compounds comprise co-stimulating molecules provided in the form of proteins or nucleic acids such as B7-1 and B7-2 (CD80 and CD86, respectively).

According to the invention, a “tumor specimen” is a sample such as a bodily sample containing tumor or cancer cells such as circulating tumor cells (CTC), in particular a tissue sample, including body fluids, and/or a cellular sample. According to the invention, a “non-tumorous specimen” is a sample such as a bodily sample not containing tumor or cancer cells such as circulating tumor cells (CTC), in particular a tissue sample, including body fluids, and/or a cellular sample. Such bodily samples may be obtained in the conventional manner such as by tissue biopsy, including punch biopsy, and by taking blood, bronchial aspirate, sputum, urine, feces or other body fluids. According to the invention, the term “sample” also includes processed samples such as fractions or isolates of biological samples, e.g. nucleic acid or cell isolates.

The immunotherapeutic agents and compositions thereof described herein may be administered via any conventional route, including by injection or infusion. The administration may be carried out, for example, orally, intravenously, intraperitoneally, intramuscularly, subcutaneously or transdermally. In one embodiment, administration is carried out intranodally such as by injection into a lymph node. Other forms of administration envision the in vitro transfection of antigen presenting cells such as dendritic cells with nucleic acids described herein followed by administration of the antigen presenting cells.

The term “pharmaceutically acceptable” refers to the non-toxicity of a material which does not interact with the action of the active component of the pharmaceutical composition.

The pharmaceutical compositions of the present invention may contain salts, buffers, preserving agents, carriers and optionally other therapeutic agents. Preferably, the pharmaceutical compositions of the present invention comprise one or more pharmaceutically acceptable carriers, diluents and/or excipients.

The term “excipient” is intended to indicate all substances in a pharmaceutical composition which are not active ingredients such as binders, lubricants, thickeners, surface active agents, preservatives, emulsifiers, buffers, flavoring agents, or colorants.

The term “diluent” relates a diluting and/or thinning agent. Moreover, the term “diluent” includes any one or more of fluid, liquid or solid suspension and/or mixing media.

The term “carrier” relates to one or more compatible solid or liquid fillers or diluents, which are suitable for an administration to a human. The term “carrier” relates to a natural or synthetic organic or inorganic component which is combined with an active component in order to facilitate the application of the active component. Preferably, carrier components are sterile liquids such as water or oils, including those which are derived from mineral oil, animals, or plants, such as peanut oil, soy bean oil, sesame oil, sunflower oil, etc. Salt solutions and aqueous dextrose and glycerin solutions may also be used as aqueous carrier compounds.

Pharmaceutically acceptable carriers or diluents for therapeutic use are well known in the pharmaceutical art, and are described, for example, in Remington's Pharmaceutical Sciences, Mack Publishing Co. (A. R Gennaro edit. 1985). Examples of suitable carriers include, for example, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Examples of suitable diluents include ethanol, glycerol and water.

Pharmaceutical carriers, excipients or diluents can be selected with regard to the intended route of administration and standard pharmaceutical practice. The pharmaceutical compositions of the present invention may comprise as, or in addition to, the carrier(s), excipient(s) or diluent(s) any suitable binder(s), lubricant(s), suspending agent(s), coating agent(s), and/or solubilizing agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

In one embodiment, the composition is an aqueous composition. The aqueous composition may optionally comprise solutes, e.g. salts. In one embodiment, the composition is in the form of a freeze-dried composition. A freeze-dried composition is obtainable by freeze-drying a respective aqueous composition.

The present invention is described in detail and is illustrated by the figures and examples, which are used only for illustration purposes and are not meant to be limiting. Owing to the description and the examples, further embodiments which are likewise included in the invention are accessible to the skilled worker.

### FIGURES

FIGS. 1a-1f are histograms showing systemic lymphocyte count (FIG. 1a), systemic platelet count (FIG. 1b), and systemic serum cytokine levels (IFN-α, IL-6, IFN-γ, IP-10) (FIGS. 1c-1f, respectively) before (pre) and after (2, 6, 24, and 48 hours) administration of different doses of a tetravalent RNA_{(LIP)} vaccine to fifteen individual patients. Depicted are mean values from duplicates. V=visit, whereas V2 represents the 1^{st} administration, V3 represents the 2^{nd} administration, V4 represents the 3^{rd} administration, V5 represents the 4^{th} administration, V6 represents the 5^{th} administration, V7 represents the 6^{th} administration, V8 represents the 7^{th} administration, and V9 represents the 8^{th} administration, respectively. Cohort I patients received only 6 administrations (V2-V7). Blood sampling and analysis was performed according to standard methods.

FIGS. 2a-2h are histograms showing the amounts of cytokine expression after incubation for 6 hours (solid dots) and after incubation for 24 hours (open dots) of isolated PBMCs with the RNA-liposome formulation. Single data points are mean values from triplicate experiments.

FIGS. 3a-3h are histograms showing the amounts of cytokine expression after incubation for 6 hours (solid dots) and after incubation for 24 hours (open dots) of whole blood with the RNA-liposome formulation. Single data points are mean values from triplicate experiments.

FIGS. 4a-4c are histograms showing the amounts of cytokine expression after incubation of the RNA-liposome formulation for 6 hours in whole blood (solid circles), whole blood enriched with pDCs (solid squares) and iDCs (solid triangles). Single data points are mean values from triplicate experiments.

FIGS. 5a-5c are histograms showing the amounts of cytokine expression after incubation of the RNA-liposome formulation for 24 hours in whole blood (solid circles), whole blood enriched with pDCs (solid squares) and pDCs (solid triangles). Single data points are mean values from triplicate experiments.

FIGS. 6a-6j are histograms showing the amounts of cytokine expression after incubation of PBMCs with small molecule agonists of TLR-7 after 24 hours, closed circles, N-(4-(4-amino-2-(2-methoxyethyl)-1H-imidazo[4,5-c]quinolin-1-yl)butyl)-N-(tetrahydro-2H-pyran-4-yl)acetamide (SM1), open circles, N-{4-[4-amino-2-(2-methoxyethyl)-1H-imidazo[4,5-c]quinolin-1-yl]butyl}-N-(1,1-dioxothietan-3-yl)acetamide (SM2).

FIGS. 7a-7j are histograms showing the amounts of cytokine expression after incubation of whole blood with small molecule agonists of TLR-7 after 24 hours, closed circles, N-(4-(4-amino-2-(2-methoxyethyl)-1H-imidazo[4,5-c]quinolin-1-yl)butyl)-N-(tetrahydro-2H-pyran-4-yl)acetamide (SM1), open circles, N-{4-[4-amino-2-(2-methoxyethyl)-1H-imidazo[4,5-c]quinolin-1-yl]butyl}-N-(1,1-dioxothietan-3-yl)acetamide (SM2).

FIGS. 8a-8e are histograms showing the level of activation of particular immune cells as measured by relative CD69 expression after incubation of PBMCs with small molecule agonists of TLR-7 after 24 hours, closed circles, N-(4-(4-amino-2-(2-methoxyethyl)-1H-imidazo[4,5-c]quinolin-1-yl)butyl)-N-(tetrahydro-2H-pyran-4-yl)acetamide (SM1), open circles, N-{4-[4-amino-2-(2-methoxyethyl)-1H-imidazo[4,5-c]quinolin-1-yl]butyl}-N-(1,1-dioxothietan-3-yl)acetamide (SM2).

FIGS. 9a-9e are histograms showing the level of activation of particular immune cells as measured by relative CD69 expression after incubation of whole blood with small molecule agonists of TLR-7 after 24 hours, closed circles, N-(4-(4-amino-2-(2-methoxyethyl)-1H-imidazo[4,5-c]quinolin-1-yl)butyl)-N-(tetrahydro-2H-pyran-4-yl)acetamide (SM1), open circles, N-{4-[4-amino-2-(2-methoxyethyl)-1H-imidazo[4,5-c]quinolin-1-yl]butyl}-N-(1,1-dioxothietan-3-yl)acetamide (SM2).

FIGS. 10a-10kk are graphs showing the level of cytokine secretion in the blood of male or female cynomolgus monkeys at time points after being intravenously administered a small molecule TLR-7 agonist, N-(4-(4-amino-2-(2-methoxyethyl)-1H-imidazo[4,5-c]quinolin-1-yl)butyl)-N-(tetrahydro-2H-pyran-4-yl)acetamide (SM1).

FIGS. 11a-11m are graphs showing the level of cytokine secretion in the blood of male cynomolgus monkeys at time points after being intravenously administered a small molecule TLR-7 agonist, N-(4-(4-amino-2-ethyl-1H-imidazo[4,5-c]quinolin-1-yl)butyl)-N-(1,1-dioxidothietan-3-yl)acetamide (SM3).

FIGS. 12a-12f are graphs showing the amounts of cytokine expression after 24 hour incubation of PBMCs isolated from different human individuals with a small molecule agonist of TLR-8, 2-ethyl-1-(4-((2-methyltetrahydrofuran-3-yl)amino)butyl)-1H-imidazo[4,5-c]quinolin-4-amine (SM4).

FIGS. 13a-13h are graphs showing the amounts of cytokine expression after 24 hour incubation of PBMCs isolated from different human individuals with a small molecule agonist of TLR-8, 1-(4-(cyclohexylamino)butyl)-2-ethyl-1H-imidazo[4,5-c]quinolin-4-amine (SM5).

### EXAMPLE 1

Within an approved interventional Phase I clinical trial (NCT02410733) fifteen human patients with malignant melanoma were treated on days 1 (V2), 8 (V3), 15 (V4), 22 (V5), 29 (V6), 36 (V7), 50 (V8) and 64 (V9) (patients 1, 2, and 3 only on days 1, 8, 15, 22, 29, 43) with increasing amounts of a nanoparticulate liposome formulated RNA-based immunotherapeutic by intravenous administration. The immunotherapeutic comprised four individual RNA-lipoplex (RNA_{(LIP)}) products, each encoding for one melanoma-associated antigen, which after intravenous administration results in efficient TLR7-triggered type-I-interferon-driven immune activation and T-cell stimulation. Patients were treated with increasing dose levels, starting with 7.2 μg total RNA for the first vaccination cycle, 14.4 μg total RNA for the second vaccination cycle, and up to 29, 50, 75 or 100 μg total RNA, respectively, for the remaining vaccination cycles.

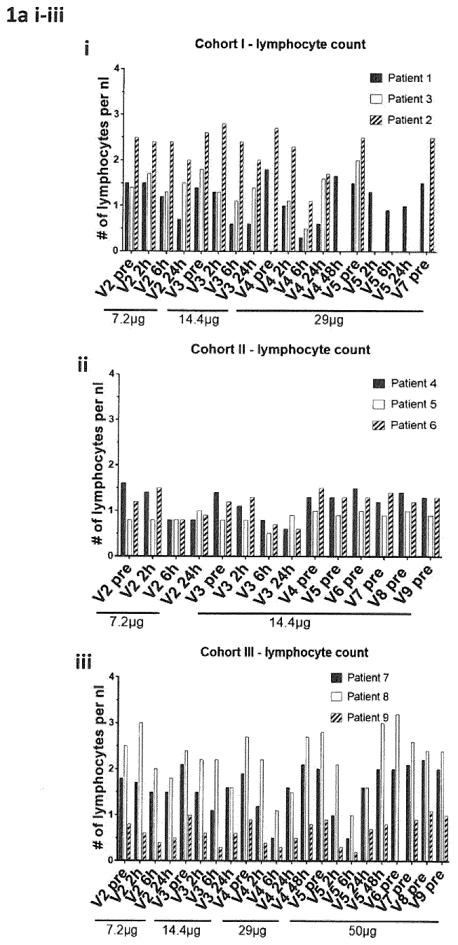

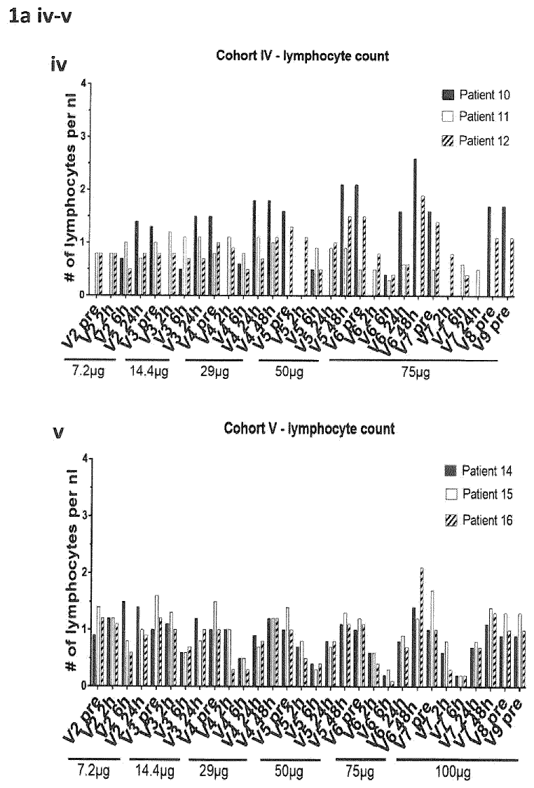

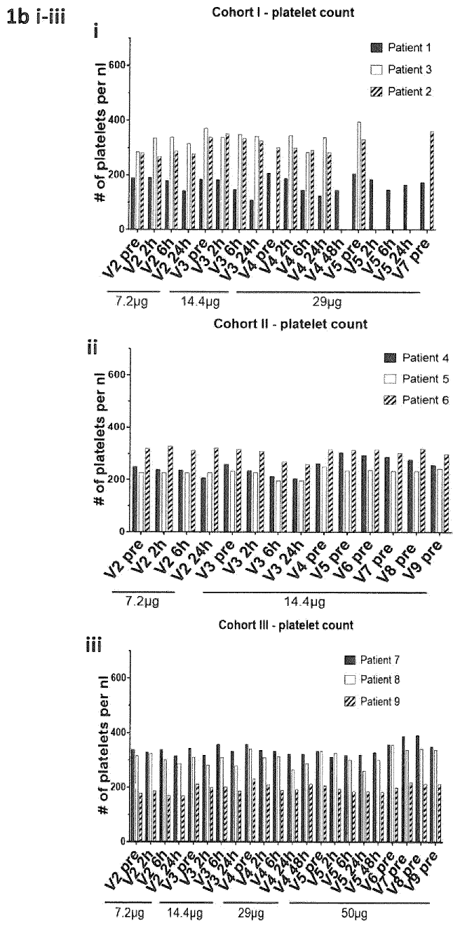

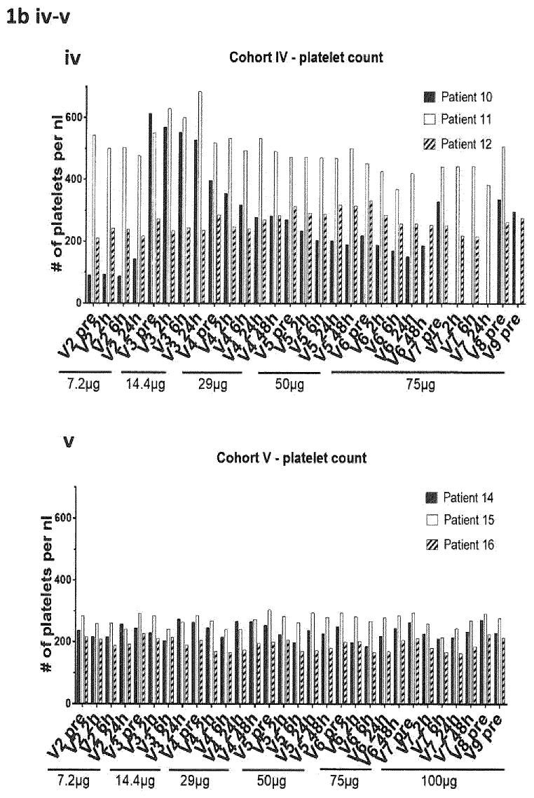

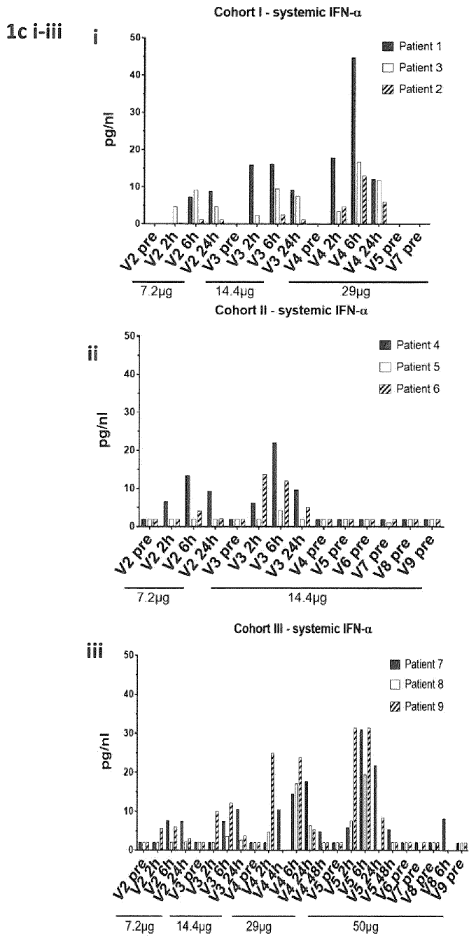

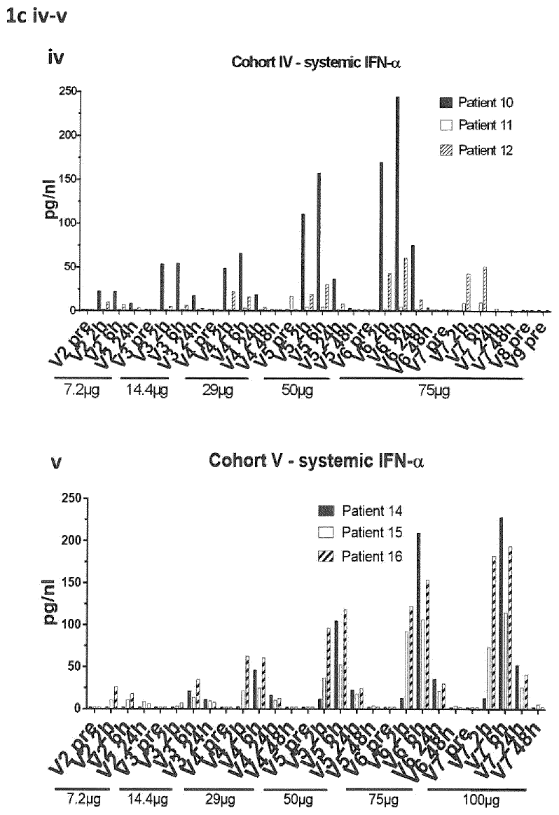

Vital signs and adverse events/serious adverse events (side effects) were assessed and reported prior to and after each vaccination cycle. Blood samples were obtained for hematological analyses and systemic cytokine measurements at individual vaccination cycles (at 0 (pre-vaccination), 2, 6, 24, and 48 hours (h) after RNA_{(LIP)} administration). Lymphocyte counts (FIG. 1a), platelet counts (FIG. 1b), and serum cytokine expression levels (FIG. 1c-1f) were determined for each patient.

The first patient (female, born 1982) experienced symptoms typically associated with immune system activation such as headache, fatigue, shivering, and fever, within hours after administration of the immunotherapeutic. These symptoms were dose-dependent and were observed at a dose of 14.4 μg (2^{nd} vaccination cycle). After treatment with 29 μg RNA (3^{rd} vaccination cycle), moderate fever-associated tachycardia and hypotension were additionally observed. The observed symptoms were readily manageable by administration of paracetamol but nevertheless led to a dose reduction to 14.4 μg total RNA for the remaining vaccination cycles for this patient. Hematological changes observed included a reversible dose-dependent reduction of systemic lymphocytes and thrombocytes, as well as a minor transient increase of systemic IFN-α, IL-6, IFN-γ and strong secretion of IP-10. These observations are in line with the believed mode of action for RNA_{(LIP)} immunotherapy, and confirm results observed from extensive preclinical studies.

The second patient (female, born 1947) tolerated administration of the immunotherapeutic (vaccination) at all three dose levels very well with no observed adverse events related to the immunotherapeutic. Moreover, only minor hematological changes were detected in addition to slight transient increases of IFN-γ and IL-6 as well as substantial IP-10 secretion in a dose-dependent manner. However, total secreted cytokine amounts were significantly less as compared to the first patient, as depicted in FIG. 1c-1f.

The third patient (male, born 1950) tolerated administration of the immunotherapeutic at all three dose levels very well with co-administration of paracetamol given prior and post administration at the discretion of the investigator. For this patient, mild fever after the 3^{rd} vaccination cycle (29 μg) that was resolved within 24 h was the only clinical symptom observed. As with the first patient, a dose-dependent transient decrease of systemic lymphocytes, although to a lesser extent, and a dose-dependent transient increase of IFN-α, IFN-γ, and IP-10 were observed, whereas amounts of systemic IL-6 were significantly higher than in the first and second patients but fully reversed within 24 h as well.

The fourth patient (female, born 1971) experienced symptoms typically associated with immune system activation such as headache, fatigue, and chills within hours after administration with either 7.2 μg, or 14.4 μg total RNA, respectively. At both dosages, a slight transient decrease of circulating lymphocytes was detected and moderate transient dose-dependent cytokine induction of IFN-γ, IP-10, and IFN-α comparable to the first patient was observed, whereas IL-6 increase after administration with 14.4 μg was slightly higher and rather comparable to cytokine levels for the third patient.

The fifth patient (male, born 1980) tolerated administration of the immunotherapeutic very well with slightly increased body temperature after the 2^{nd} vaccination cycle (14.4 μg) and mild joint pain after the 3′ vaccination cycle (14.4 μg). Whereas platelet count was not significantly affected, a moderate but fully transient lymphocytopenia was observed after the 2^{nd} vaccination cycle with 14.4 μg. Almost no increase of systemic cytokines was observed except for a marginal and fully reversible increase of IP-10 secretion after the 2^{nd} vaccination cycle that was the lowest in comparison to the other five patients.

The sixth patient (female, born 1974) tolerated administration of the immunotherapeutic very well. With this patient, chills, headache, and pain in the limbs (all mild) were the only clinical observed adverse events reported upon administration of 14.4 μg (2^{nd} vaccination cycle). In addition, fully reversible slight dose-dependent decreases of platelets and lymphocytes were observed. Further, minor dose-dependent increases of systemic IFN-α and IFN-γ after the 2^{nd} vaccination cycle (14.4 μg) were detected, the latter comparable to the first and fourth patients in intensity.

Likewise, inter-individual sensitivities towards RNA_{(LIP)} treatment was observed for patients 7, 8, 9, 10, 11, 12, 14, 15, and 16 (age range 27-75 years). This is reflected by the different intensities of hematological changes and the varying transient induction of systemic cytokine levels especially at doses ≥29 μg total RNA and the diverging adverse event profiles related to repetitive RNA_{(LIP)} dosing. Whereas the majority of patients tolerated repetitive RNA_{(LIP)} very well also at doses up to 75 and 100 μg total RNA, selected patients experienced severe fever after treatment with 100 μg RNA_{(LIP)} (patient 16) or worsening of hypertension after treatment with 7.2 μg (patient 11), 14.4 μg (patient 10), and 75 or 100 μg total RNA_{(LIP)} (patient 16).

Based not only on the differences between the observed adverse events experienced at each dose among the fifteen patients, but also on the inter-individual differences in hematological changes and serum cytokine expression levels at various time points after administration of the immunotherapeutic, the data clearly indicate that different individuals vary in their intensity of induced immunological reactions upon the administration of the same immunotherapeutic agent at the same dose.

### EXAMPLE 2

The objective of the following study was to obtain information about the in vitro activation of isolated peripheral blood mononuclear cells (PBMCs) or cells in whole blood by measuring the expression of certain cytokines after the cells have been contacted with RNA molecules complexed with liposomes, which RNA molecules encode certain tumor antigens (RNA_{LIP}). The encoded antigens were NY-ESO1, a cancer antigen expressed in a wide variety of tumors (RBL001.1), tyrosinase (RBL002.2), MAGE-A3, a melanoma associated antigen (RBL003.1) and TPTE, a tyrosine-protein phosphatase (RBL004.1).

In the first part of this study, samples of human heparinized whole blood and PBMCs (isolated from heparinized whole blood) obtained from healthy donors were contacted with a mixture of equal portions of liposomally formulated RBL001.1, RBL002.2, RBL003.1 and RBL004.1. To obtain information about the role of dendritic cells (DCs) in relation to cytokine secretion in whole blood, in the second part of the study, heparinized whole blood was enriched with plasmacytoid DCs (pDCs) or monocyte-derived immature DCs (iDCs) and subsequently incubated with the liposomally formulated RNA molecules (RNA-LIP).

As a primary endpoint in both parts of this study, the activation of these cells was determined 6 h and 24 h after contacting by analysis of cytokine expression in culture supernatant. For first part of the study, the following cytokines have been analyzed: IP-10, IFN-γ, TNF-α, IL-1β, IL-2, IL-6, IL-12 and IFN-α2. In the second part of the study, only IP-10, IL-6 and IFN-α2 were analyzed.

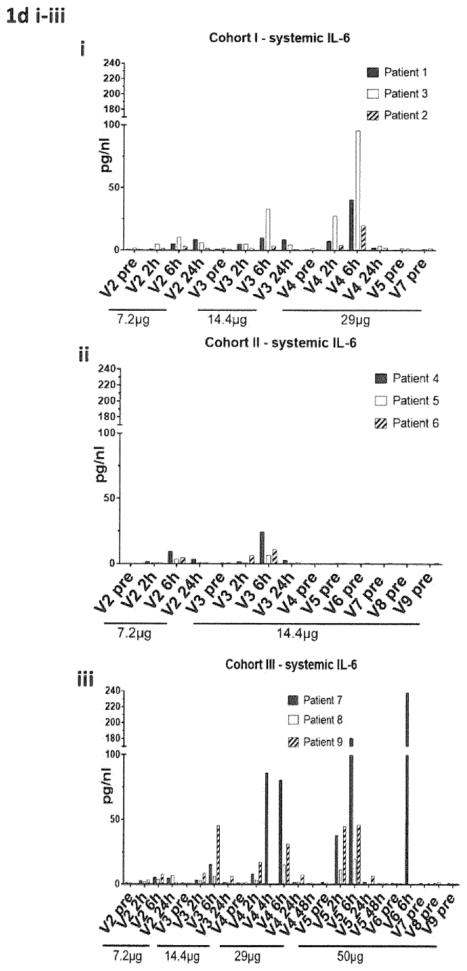

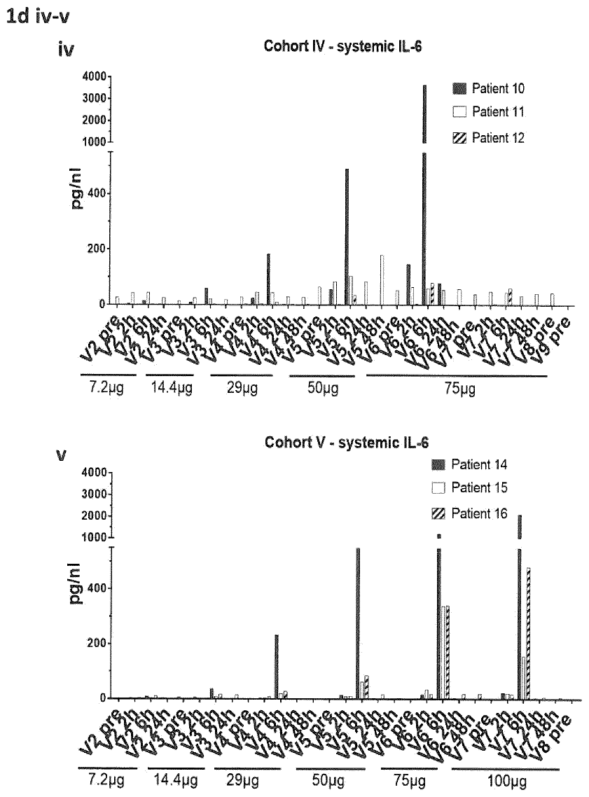

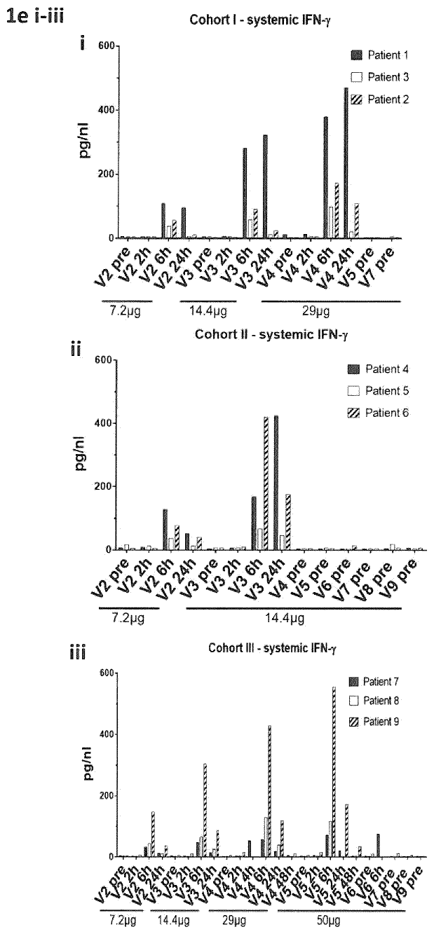

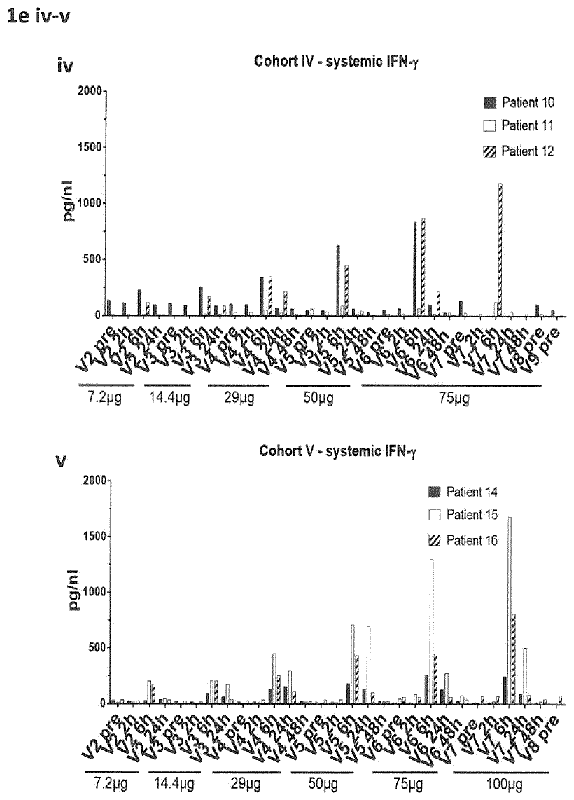

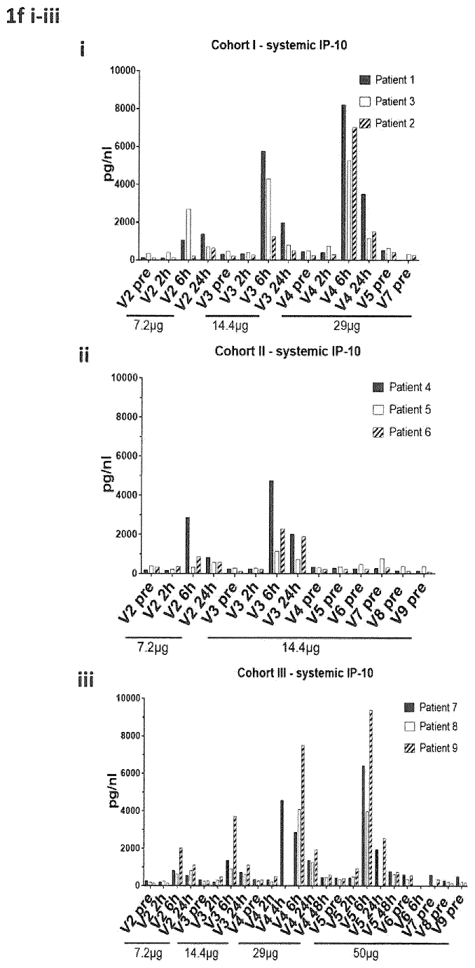

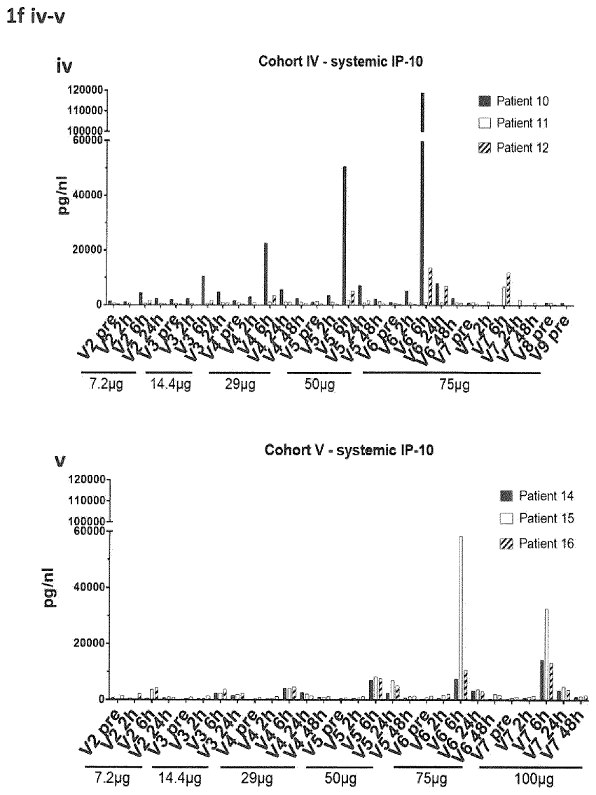

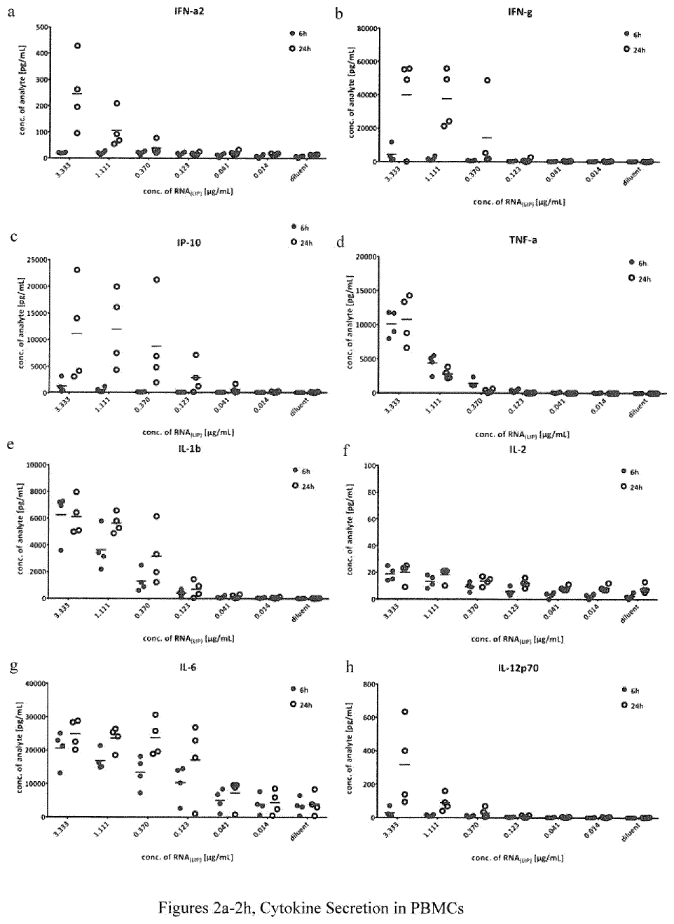

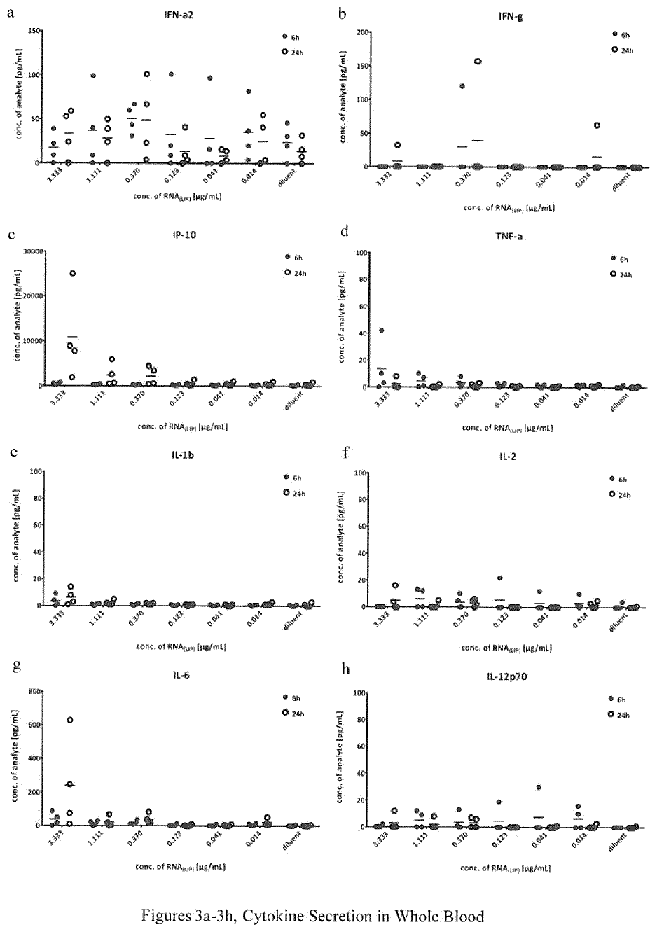

In cultured PBMCs contacted with the four different RNA-LIP compositions, a dose-dependent induction of all detectable cytokines was observed (FIGS. 2a-2h). As seen in FIGS. 3a-3h, in whole blood, where expression was able to be detected, there was no alteration in the levels of cytokines IFN-γ, TNF-α, IL-1β, IL-2, and IL-12. Also, IFN-α2 was not significantly increased at any dose in whole blood compared to diluent control. However, the chemokine IP-10 (CXCL10) was observed to be up-regulated in a dose-dependent manner in whole blood. Increased induction of IL-6 in whole blood was observed in cells obtained from only one of the four donors and only at a low level.

In the study with dendritic cell-enriched whole blood, a dose-dependent induction of the IP-10, IFN-α2, and IL-6 was also observed.

The results observed from isolated PBMCs showed some differences compared to whole blood suggesting a higher sensitivity of the test system with isolated PBMCs. With isolated PBMCs, increased cytokine levels were observed for cytokines tested, whereas with PBMCs in whole blood, cytokine detection was restricted to IFN-α2, IP-10, and IL-6. In the second part of the study, cytokine secretion in whole blood was increased by enrichment with different types of DCs, suggesting that DCs are a major cell type for uptake of the RNA-liposome complexes. Based on this data it could be assumed that pDCs are the major cell type for liposomally formulated RNA uptake in whole blood since enrichment with fresh pDCs lead to increased IFN-α2 secretion.

Moreover, these results clearly show significant individual variations in various immunological reactions in response to contacting an immunotherapeutic agent (RNA-LIP) with immune-reactive material of an individual (PBMCs, whole blood, and whole blood enriched with DCs) at the same dose.

Materials:

The materials used in the studies and their individual sources are as follows: Customer 7-plex (Cat.: L5002JFHHC), IFN-α2 single Plex (Cat.: 171-B6010M), IP-10 single Plex (Cat.: 171-B5020M), IL-6 single Plex (Cat.: 171-B5006M), Cytokine Standards Group II (Cat.: 171-D60001), Cytokine Standards Group I (Cat.: 171-D50001), Bio-Plex Pro Reagent Kit (Cat.: 171-304070M), all obtained from Bio-Rad Laboratories GmbH; Sodium Pyruvate (Cat.: 11360-039), Non-Essential Amino Acids (Cat.: 11140-035), Penicillin-Streptomycin (Cat.: 15140-122), HEPES (Cat.: 15360-056), RPMI-1640+ Glutamax™ (Cat.: 61870-010), all obtained from Invitrogen, GIBCO®; Human Serum Type AB obtained from LONZA; IL-4 (Cat. No.: 130-093-924), CD14-Beads (Cat.: 130-050-201), CD304-Beads (Cat.: 130-090-532), all obtained from Miltenyi Biotech GmbH; Leucomax, Molgramostim (rHuman GM-CSF) obtained from Novartis; and Ficoll-Paque PLUS (Cat.: 17-1440-03) obtained from GE Healthcare.

Methods:

Whole blood was collected from healthy volunteers in sterile syringes. Heparin was used as anticoagulant. Heparinized whole blood was used to generate PBMCs by density centrifugation on Ficoll-Paque. iDCs were isolated by using freshly prepared PBMCs isolated from whole blood and the isolation of CD14+ monocytes was by magnetic bead based separation. pDCs were isolated by using freshly prepared PBMCs isolated from whole blood and the isolation of pDCs was by magnetic bead based separation.

For the first part of this study, heparinized whole blood was collected from healthy donors (n=4). One part of whole blood was used to generate PBMCs. Subsequently, PBMCs were resuspended in medium and seeded in a 96-well-plate. In detail, per dose group 5×10⁵ PBMCs were seeded in 180 μL per well. Then 20 μL of the solutions containing the liposome-complexed RNA (RNA-liposome mixture) was added to reach a final volume of 200 μL (1:10 dilution of each solution) and a final cell density of 2.5×10⁶ PBMCs/mL. For all doses tested, data was generated from triplicates. The remaining whole blood obtained from the same donors was pipetted directly into the wells of a 96-well-plate. In detail, 1804 whole blood was seeded in triplicates for all dose groups and 20 μL of the solutions containing the liposome-complexed RNA was added to reach a final volume of 200 μL and a 1:10 dilution of spike solutions. Tables 1 and 2 below summarize the individual test samples:

TABLE 1
Dose groups for the first part of the study (isolated PBMCs)

| | | | Final Dose |
|---|---|---|---|
| Dose Group | Test System | Test Item/IVT RNA | (μg RNA/ml) |
| #1 | PBMCs | RNA-liposome mixture | 3.33 |
| #2 | PBMCs | RNA-liposome mixture | 1.111 |
| #3 | PBMCs | RNA-liposome mixture | 0.370 |
| #4 | PBMCs | RNA-liposome mixture | 0.123 |
| #5 | PBMCs | RNA-liposome mixture | 0.041 |
| #6 | PBMCs | RNA-liposome mixture | 0.014 |
| ctrl | PBMCs | Diluent/NaCl (0.9%) | 0 |

TABLE 2
Dose groups for the first part of the study (whole blood)

| | | | Final Dose |
|---|---|---|---|
| Dose Group | Test System | Test Item/IVT RNA | (μg RNA/ml) |
| #1 | Whole blood | RNA-liposome mixture | 3.33 |
| #2 | Whole blood | RNA-liposome mixture | 1.111 |
| #3 | Whole blood | RNA-liposome mixture | 0.370 |
| #4 | Whole blood | RNA-liposome mixture | 0.123 |
| #5 | Whole blood | RNA-liposome mixture | 0.041 |
| #6 | Whole blood | RNA-liposome mixture | 0.014 |
| ctrl | Whole blood | Diluent/NaCl (0.9%) | 0 |

For the second part of the study, heparinized whole blood was collected twice from the same donors (n=2). The first time, heparinized whole blood was used to generate PBMCs and subsequently to isolate CD14+ monocytes. The isolated monocytes were cultivated for five days to generate iDCs. Cytokines IL-4 and GM-CSF (1000 U/ml of each) were added to the culture medium, to stimulate the generation of iDCs. After three days, the cells were fed with fresh medium including cytokines. Then, heparinized whole blood from the same donors was collected for a second time. One part of this blood was used to generate PBMCs and subsequently to isolate pDCs. Afterwards, the rest of whole blood was seeded into wells as described above: 180 μL per well in a 96-well-plate. For the highest dose group, 100 μL whole blood was pipetted. Adding autologous pDCs or iDCs was as follows: 10,000 DCs were added to whole blood samples as indicated in Table 3. After addition of DCs, 20 μL of solutions containing the liposomally formulated RNAs were added to reach a final volume of 200 μL and a 1:10 dilution of the solutions.

TABLE 3
Dose groups for the second part of the study

| | | Spiking with 10,000 | | Final Dose |
|---|---|---|---|---|
| Dose Group | Test System | DCs (type) | Test Item/IVT RNA | (μg RNA/ml) |
| #1 | Whole blood | — | RNA-liposome mixture | 50 |
| #2 | Whole blood | — | RNA-liposome mixture | 10 |
| #3 | Whole blood | — | RNA-liposome mixture | 2 |
| #4 | Whole blood | — | RNA-liposome mixture | 0.4 |
| #5 | Whole blood | — | RNA-liposome mixture | 0.08 |
| #6 | Whole blood | — | RNA-liposome mixture | 0.016 |
| ctrl | Whole blood | — | Diluent/NaCl (0.9%) | 0 |
| #1 | Whole blood | iDCs | RNA-liposome mixture | 50 |
| #2 | Whole blood | iDCs | RNA-liposome mixture | 10 |
| #3 | Whole blood | iDCs | RNA-liposome mixture | 2 |
| #4 | Whole blood | iDCs | RNA-liposome mixture | 0.4 |
| #5 | Whole blood | iDCs | RNA-liposome mixture | 0.08 |
| #6 | Whole blood | iDCs | RNA-liposome mixture | 0.016 |
| ctrl | Whole blood | iDCs | Diluent/NaCl (0.9%) | 0 |
| #1 | Whole blood | pDCs | RNA-liposome mixture | 50 |
| #2 | Whole blood | pDCs | RNA-liposome mixture | 10 |
| #3 | Whole blood | pDCs | RNA-liposome mixture | 2 |
| #4 | Whole blood | pDCs | RNA-liposome mixture | 0.4 |
| #5 | Whole blood | pDCs | RNA-liposome mixture | 0.08 |
| #6 | Whole blood | pDCs | RNA-liposome mixture | 0.016 |
| ctrl | Whole blood | pDCs | Diluent/NaCl (0.9%) | 0 |

Experimental Timeline

First part of study:

Day 1: Collection of heparinized whole blood (n=4)

Preparation of PBMCs of each donor

Seed PBMCs and whole blood

Addition of solutions of RNA-LIP and incubate

Harvest supernatants/plasma at the 6 h time point and freeze at −65 to −85° C.

Day 2: Harvest supernatants/plasma at the 24 h time point and freeze at −65 to −85° C.

Perform analysis of the frozen supernatant on any following day

Second part of study:

Day 1: Collection of whole blood (n=2)

Preparation of PBMCs from each donor

Isolation of CD14+ monocytes from PBMCs

Cultivation of isolated CD14+ monocytes to generate iDCs

Day 4: Feeding iDCs

Day 6: Harvest iDCs

Collection of whole blood (n=2; same donors)

Preparation of PBMCs of each donor

Isolation of pDCs from PBMCs

Seed whole blood

Adding iDCs or pDCs, respectively

Addition of solutions containing RNA-LIP

Harvest supernatants/plasma at the 6 h time point and freeze at −65 to −85° C.

Day 7: Harvest supernatants/plasma at the 24 h time point and freeze at −65 to −85° C.

Perform analysis of the supernatants/plasma on any following day

Results:

After incubation of PBMCs with the RNA-LIP mixture for 24 h, a dose-dependent secretion of all cytokines was detected. However, high variations in concentration levels of the cytokines (20-60,000 pg/ml) were observed. The cytokine response was dominated by five out of the eight selected cytokines, namely IP-10, IFN-γ, TNF-α, IL-1β, and IL-6 (see Table 4). Additionally, differences in the time point of secretion were also observed: IL-1β, IL-6 and TNF-α already were detected after 6 h of incubation (at high RNA concentrations) and the levels were not increased remarkably after 24 h, indicating variation in the ability of cells from the different donors to respond to the addition of the RNA-LIP composition.

TABLE 4
Summary of Results from Isolated PBMCs

| Cytokine | Result |
|---|---|
| IFN-α2 | Elevated secretion detectable after 24 h in |
| | samples obtained from all donors |
| | Dose-dependent induction |
| | Values on a low level and not elevated |
| | remarkably in doses 0.014-0.37 μg/mL |
| IP-10 | Elevated secretion detectable after 24 h in |
| | samples obtained from all donors |
| | Dose-dependent induction |
| | In 4/4 donors elevated levels detectable |
| | at 0.37 μg/mL |
| | In 3/4 donors elevated levels detectable |
| | at 0.12 μg/mL |
| IL-6 | Elevated secretion detectable after 6 h and |
| | 24 h in samples obtained from all donors |
| | Dose-dependent induction |
| | In 4/4 donors elevated levels detectable at |
| | 0.37 μg/mL |
| | In 3/4 donors elevated levels detectable at |
| | 0.12 μg/mL after 24 h |
| IFN-γ | Elevated secretion detectable after 24 h in |
| | samples obtained from all donors |
| | Dose-dependent induction |
| | In 4/4 donors elevated levels detectable at |
| | 0.37 μg/mL |
| TNF-α | Elevated secretion detectable after 6 h and |
| | after 24 h in samples obtained from all donors |
| | Dose-dependent induction |
| | In 4/4 donors elevated levels detectable at |
| | 0.37 μg/mL only after 6 h |
| IL-1β | Elevated secretion detectable after 6 h and |
| | 24 h in all donors |
| | Dose-dependent induction |
| | In 4/4 donors elevated levels detectable at |
| | 0.37 μg/mL |
| | In 2/4 donors elevated levels detectable |
| | at 0.12 μg/mL after 24 h |
| IL-2 | Elevated secretion detectable after 6 h and |
| | after 24 h in samples obtained from all donors |
| | Dose-dependent induction |
| | Values on a low level and not elevated in |
| | doses 0.014-0.37 μg/mL |
| IL-12 | Elevated secretion detectable after 24 h in |
| | samples obtained from all donors |
| | Dose-dependent induction |
| | In 2/4 donors elevated levels detectable at |
| | 0.37 μg/mL |

In addition to studies with isolated PBMCs, analogous experiments in whole blood were performed. In these studies, no elevated cytokine secretion to a remarkable concentration was detectable after incubation with the RNA-LIP composition for the following cytokines: IFN-γ, TNF-α, IL-1β, IL-2, and IL-12 (see Table 5 and FIGS. 3a-3h). Note, that at some data points, elevated levels that were observed only in 1 out of 3 replicates were considered outliers. Regarding IFN-α2, there was a low level baseline secretion detectable in some samples of three out of four donors, including the diluent control. Elevated secretion of IP-10 was detected for all donors after 24 h and at the highest dose tested. In two out of four donors, there were still elevated levels detectable at dose #3 (0.37 μg/mL). Although on a very low level, IL-6 was elevated after addition of RNA-LIP in two out of four donors after 24 h and at the highest dose tested.

TABLE 5
Summary of Results for Whole Blood

| Cytokine | Result |
|---|---|
| IFN-α2 | Secretion detectable on a very low level in |
| | some samples obtained from three of four donors |
| | No dose-dependency and no distinct |
| | elevation detectable compared to control in |
| | any dilution |
| IP-10 | Elevated secretion detectable after 24 h in |
| | samples obtained from all donors |
| | Dose-dependent induction |
| | In samples obtained from two out of the four |
| | donors, elevated levels were detectable at |
| | 0.37 μg/mL |
| IL-6 | Elevated secretion detectable at a very low |
| | level and only at highest dose in samples |
| | obtained from two out of the four donors |
| IFN-γ | No elevated secretion detectable |
| TNF-α | No elevated secretion detectable |
| IL-1β | No elevated secretion detectable |
| IL-2 | No elevated secretion detectable |
| IL-12 | No elevated secretion detectable |

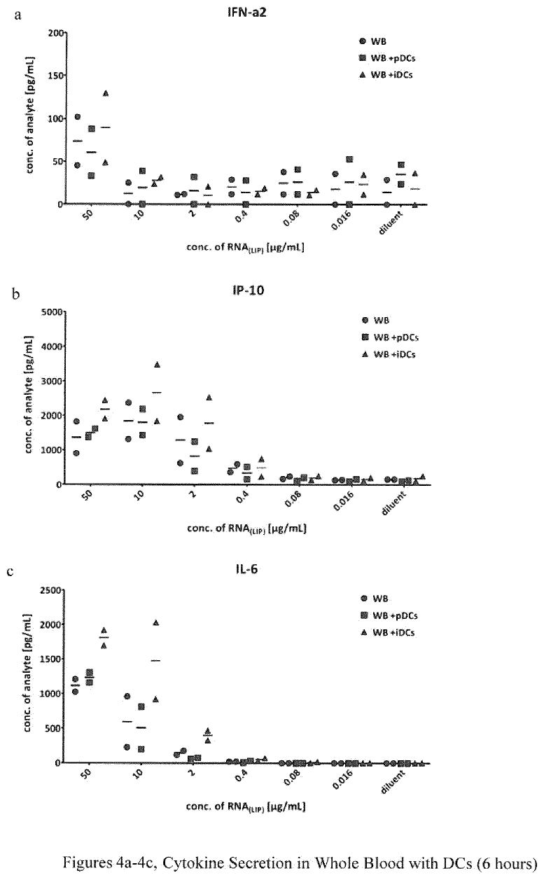

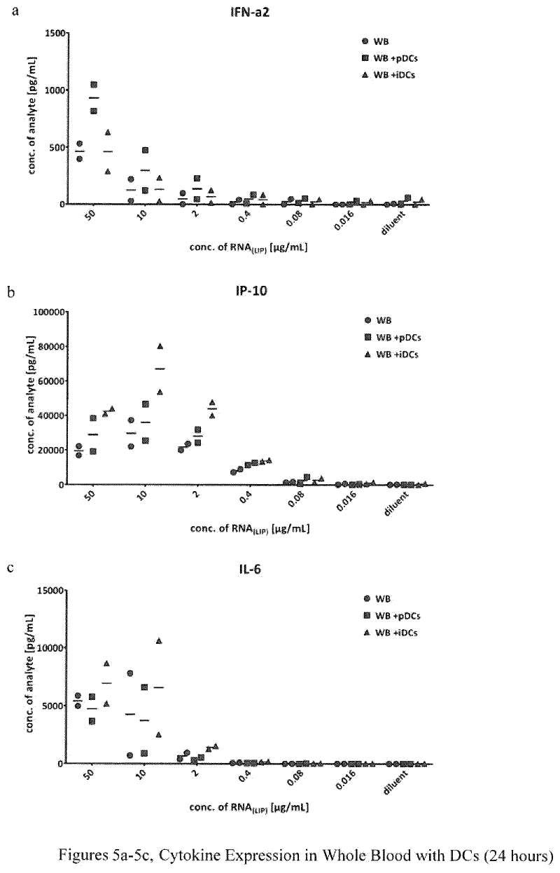

To test the role of DCs for activation and cytokine secretion in whole blood, the second part of the study was performed. Here, heparinized whole blood, enriched with autologous iDCs or pDCs, was incubated with an RNA-LIP composition in a dose range of 0.016 to 50 μg/mL. The results presented in FIGS. 4a-4c and FIGS. 5a-5c show a clear dose-dependent induction of IFN-α2, IP-10 and IL-6. With regard to IFN-α2, elevated secretion was observed after 24 h incubation with the RNA-LIP composition in whole blood only at the two highest doses tested (10 and 50 μg/mL). With dose 3 (2 μg/mL), there was no increased secretion detectable. However addition of pDCs to the heparinized whole blood increased the secretion of IFN-α2 at the 2 μg/mL dose. In contrast, addition of iDCs did not lead to increased secretion of this cytokine compared to whole blood alone. Remarkably, elevated levels of IP-10 were detected after 24 h incubation with 0.4 to 50 μg/mL RNA-LIP (slightly with 0.08 μg/mL), which confirmed the results from the first part of the study. Addition of either type of DC led to an even greater secretion of the detected cytokines compared to no addition, although the highest levels were detected in whole blood enriched with iDCs. Regarding IL-6, the results from the first part of the study were confirmed, i.e., increased expression levels were detected with doses higher than 2 μg/mL. Here, addition of iDCs to the whole blood also led to an increased level of expression compared to whole blood alone.

Discussion and Conclusion

Regarding the first part of the study, in both test systems, isolated human PBMCs and whole blood, several cytokines were secreted after incubation with the RNA-LIP composition. However, differences between the test systems suggest that PBMCs have a higher sensitivity as test system. On the one hand, increased cytokine levels in isolated PBMCs for all eight tested cytokines could be detected. Using whole blood as a test system, cytokine detection was restricted to IFN-α2, IP-10, and IL-6. The differing results could be caused by different cultivation conditions since isolated PBMCs were cultivated with culture medium supplemented with serum (FCS) and antibiotics whereas cultivation in whole blood means that PBMCs were cultured with human plasma and red blood cells.

In the second part of the study it was shown that IFN-α2 secretion in whole blood could be increased with addition of pDCs. It is known that pDCs secrete IFN-α upon TLR-7 activation (Kwissa et al., 2012, Distinct TLR adjuvants differentially stimulate systemic and local innate immune responses in nonhuman primates, Blood 119:2044-2055; Schiller et al., 2006, Immune response modifiers—mode of action, Exp. Dermatol. 15:331-341, Hornung et al., 2005, Sequence-specific potent induction of IFN-alpha by short interfering RNA in plasmacytoid dendritic cells through TLR7, Nat. Med. 11(3):263-270). Since TLR-7 is activated by RNA within endosomes, this indicates that pDCs are a major cell type for uptake of RNA.

The results from these experiments also show that the same immune-reactive material isolated from different individuals contacted with the same immunotherapeutic agent at the same concentration (same dose) results in a significantly different immunological reaction, thus further supporting the conclusion that the immunotherapeutic agents at the same dose have different effects in different individuals such and that there is no single dose that is therapeutically effective and/or tolerated in all individuals.

### EXAMPLE 3

The objective of the following study was to obtain information about the activation in vitro of isolated peripheral blood mononuclear cells (PBMCs) or cells in fresh whole blood by measuring the secretion of certain cytokines and the induction of a general activation marker (CD69) after the cells were incubated with various concentrations of small TLR-7 agonist compounds, namely N-(4-(4-amino-2-(2-methoxyethyl)-1H-imidazo[4,5-c]quinolin-1-yl)butyl)-N-(tetrahydro-2H-pyran-4-yl)acetamide (designated herein as SM1) and N-{4-[4-amino-2-(2-methoxyethyl)-1H-imidazo[4,5-c]quinolin-1-yl]butyl}-N-(1,1-dioxothietan-3-yl)acetamide (designated herein as SM2). As discussed in more detail below, human heparinized whole blood and PBMCs (isolated from buffy coats) obtained from healthy donors were incubated for 24 hours with equimolar amounts of agonist compounds SM1 or SM2.

PBMCs used for the experiment were isolated from buffy coats by density centrifugation on Ficoll-Paque. Subsequently, PBMCs were resuspended in cell culture medium and seeded in a 96-well-plate. In detail, per dose group 5×10⁵ PBMCs were seeded in 1900 per well. Then 10 μL of agonists at specified concentrations was added to reach a final volume of 200 μL (1:20-dilution of each solution) and a final cell density of 2.5×10⁶ PBMCs/mL. Plates were incubated at 37° C. and 5% CO₂ for 24 hours. Subsequently, supernatants were harvested and immediately analyzed or frozen and kept at −80° C. until analysis. For all doses of the agonist tested, data was generated from 10 individuals (for both study parts), each in biological duplicates.

Whole blood was collected from healthy volunteers in sterile syringes. Heparin was used as anticoagulant. The fresh heparinized whole blood was pipetted directly into a 96-well-plate. In detail, 190 μL whole blood from different individuals (n=10 donors for CBA; n=8 for flow cytometry) was seeded in duplicates for all dose groups and 10 μL of test-item-solutions was added to reach a final volume of 200 μL and a 1:20 dilution of spike solutions. Plates were incubated at 37° C. and 5% CO₂ for 24 hours. Subsequently, supernatants were harvested and immediately analyzed or frozen and kept at −80° C. until analysis.

For measuring cellular activation by CD69 expression, cell pellets were harvested and flow cytometry staining and measurements were performed immediately.

Each agonist was prepared in a serial dilution with the diluent DMSO (dimethyl sulfoxide): 5-fold in 8 steps. Agonist compound concentrations incubated with the PBMCs or whole blood were 10 μM, 2 μM, 0.4 μM, 0.08 μM, 0.016 μM, 0.0032 μM, 0.0006 μM, and 0.0001 μM.

As primary endpoint of the first part of the study, activation of the cells was determined by analysis of induction of cytokine secretion in cell culture supernatant (PBMCs) and plasma (whole blood) via cytometric bead assay (CBA). The following cytokines/chemokines were analyzed (IFN-α, IP-10, IFN-γ, TNF-α, IL-6, IL-8, IL-10, IL-12p70 and IL-2). For the second part of the study, cellular activation was determined by analysis of the expression of the general activation marker CD69 in several types of immune cells via flow cytometry. The following types of immune cells were investigated: plasmacytoid dendritic cells (pDCs), myeloid dendritic cells (mDCs), monocytes, B cells and NK cells.

In particular, for determination of cytokine concentrations a cytometric bead assay (Multiplex-Kit, (ProcartaPlex; eBioscience), was used, which included all ten cytokines/chemokines (IFN-α, IP-10, IFN-γ, TNF-α, IL-6, IL-8, IL-10, IL-12p70 and IL-2). Analysis was performed with a Luminex® system.

For flow cytometry cell pellets were stained with an antibody mixture, combining the surface markers, CD3, CD16, CD19, CD14, BDCA2 and BDCA3, and the activation marker CD69. With this flow panel it was possible to analyze the activation of B cells, NK cells, monocytes, plasmacytoid dendritic cells (pDCs) and myeloid dendritic cells (mDCs). Measurements were performed with the BD FACSCanto II™.

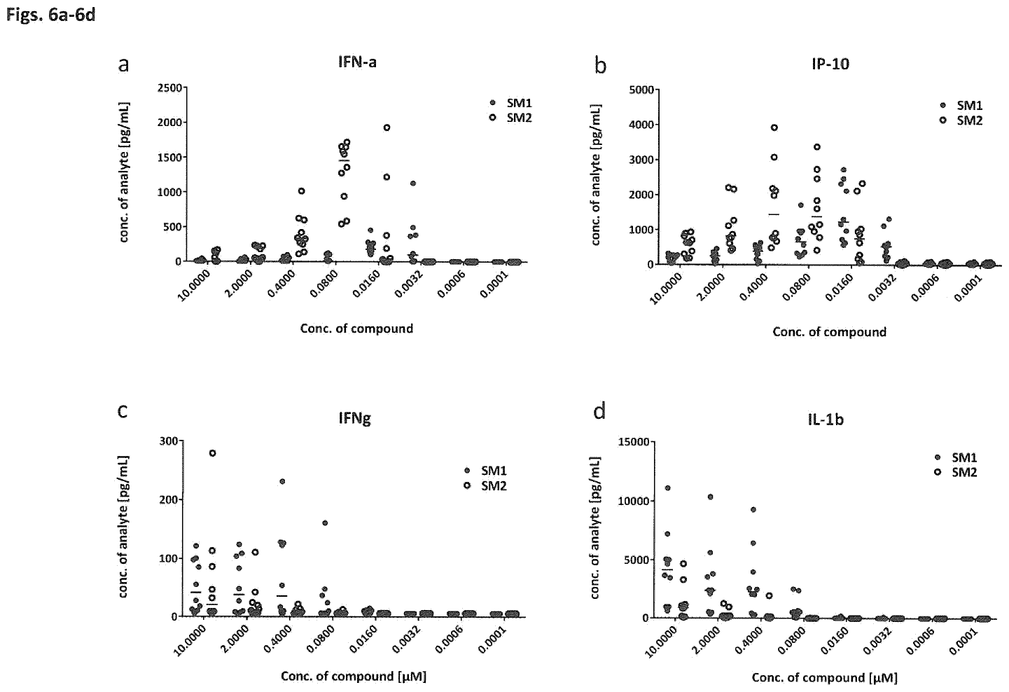

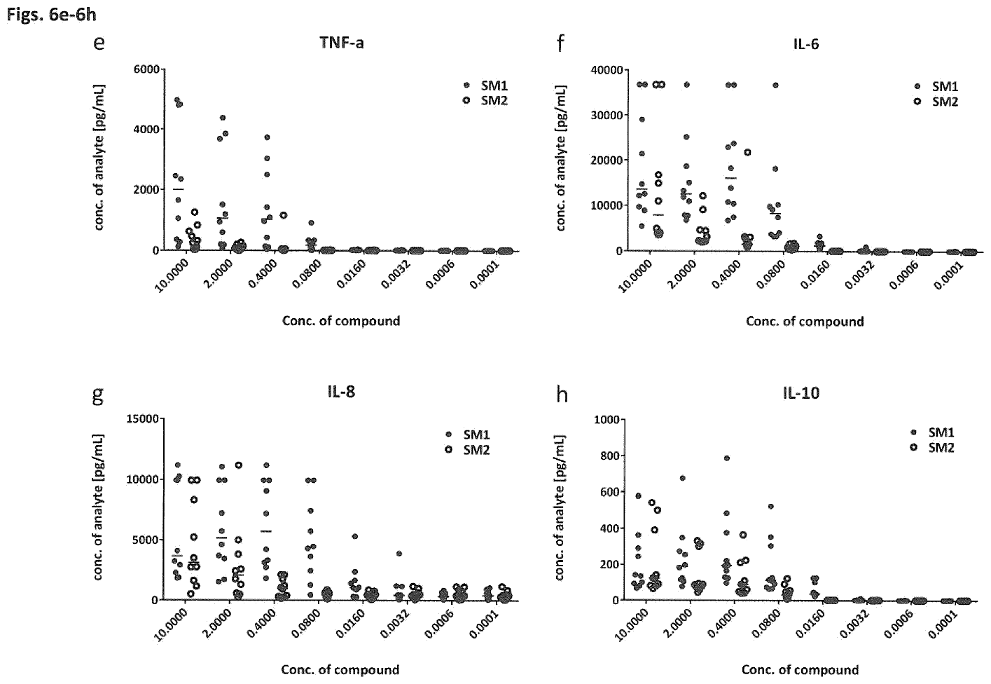

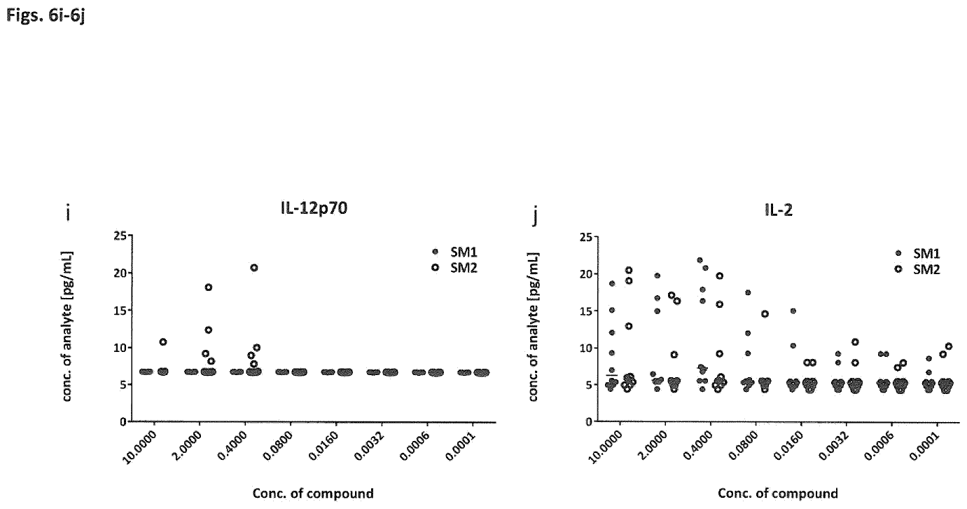

In cultured PBMCs incubated with the TLR7-agonists, a consistent and dose-dependent induction of 8 out of 10 measured cytokines was observed (FIGS. 6a-6h). Only IL-12p70 and IL-2 were not induced consistently, and in the few cases they were only expressed in a low level in PBMCs of some donors (FIGS. 6i-6j). Comparing the cytokine levels of different donors at different agonist concentrations shows a high variability for both agonist compounds, which is attributed to the high inter-individual responsiveness of the immune system to external stimuli, as exemplified by the incubation of exemplary TLR7 agonists with immune cells in an in vitro setting.

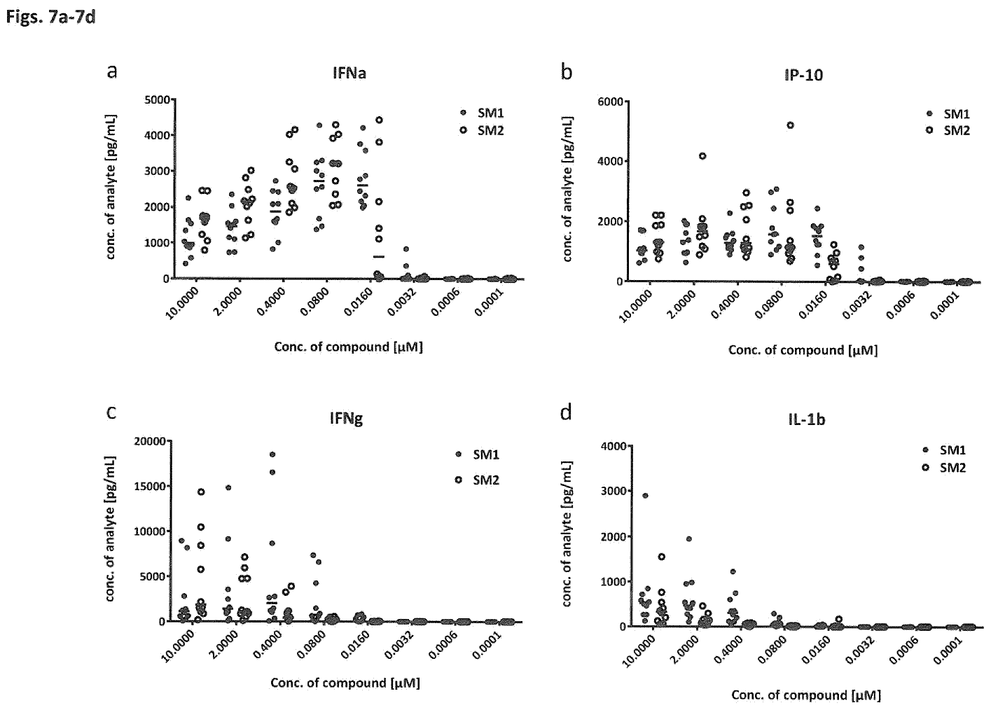

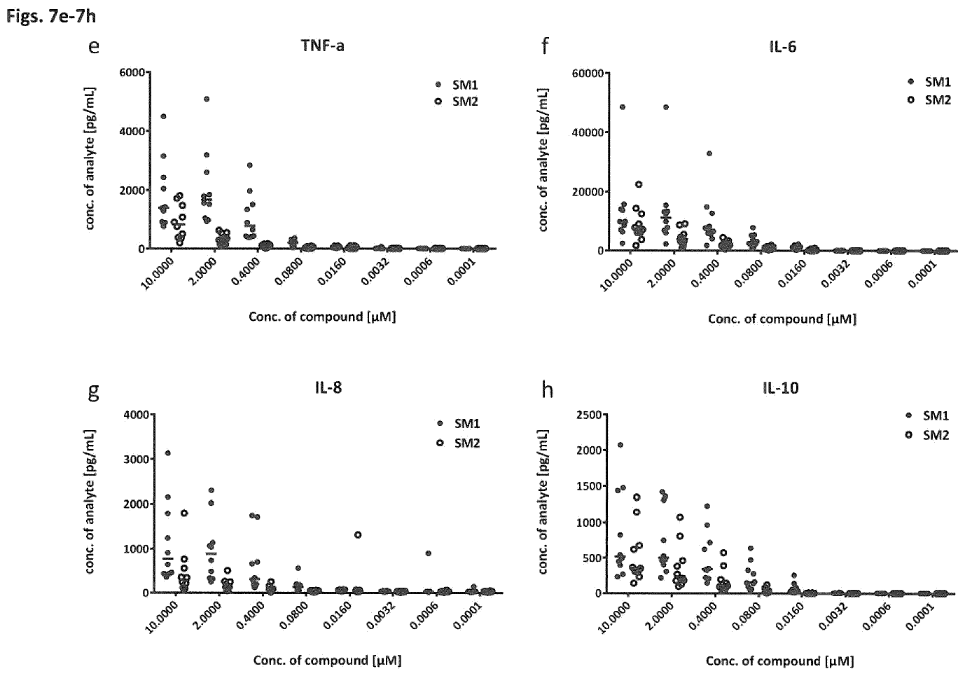

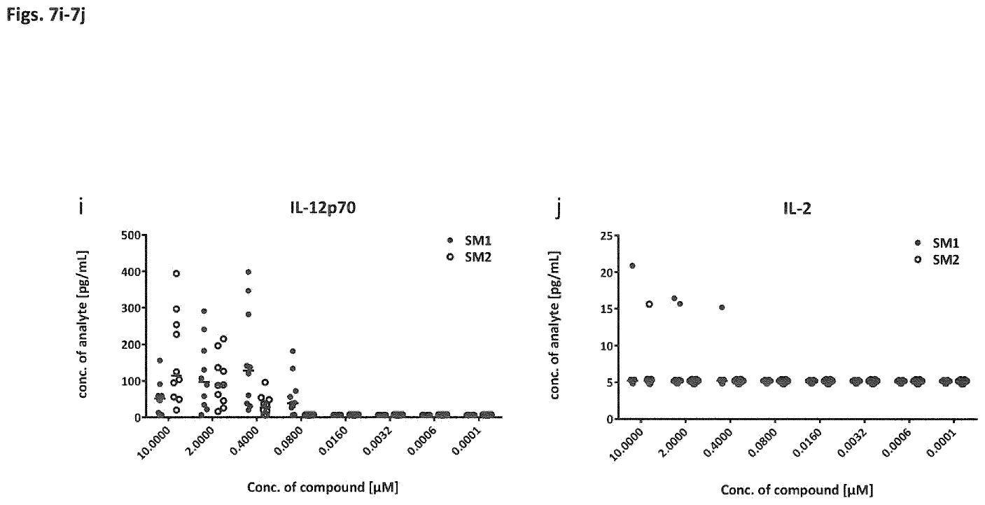

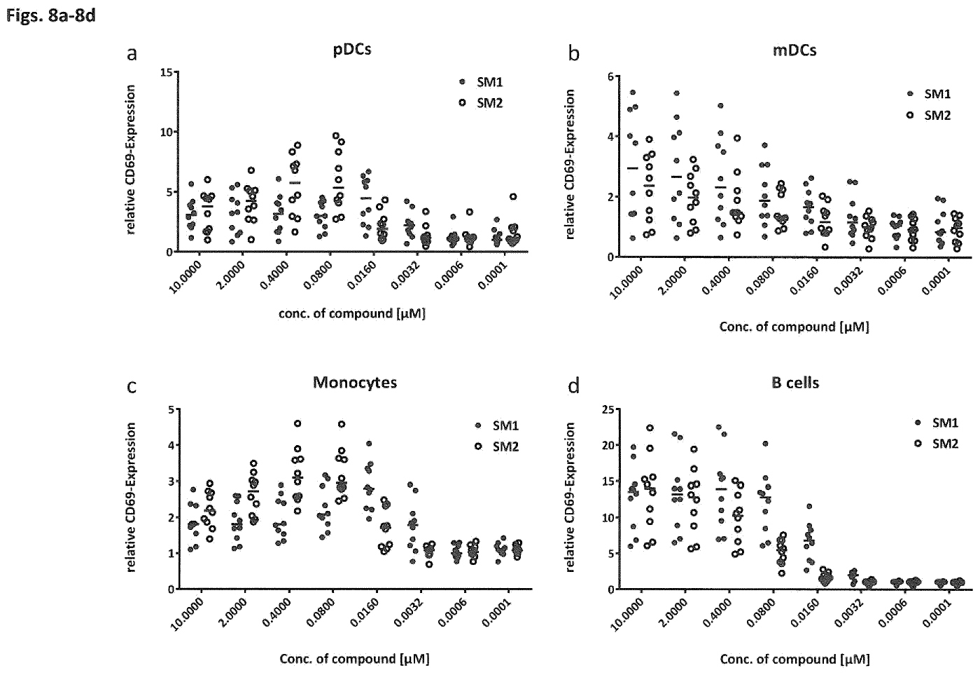

After incubation of whole blood with each agonist compound (FIGS. 7a-7j), the dose-response-curves for the detected cytokines are mostly comparable to the observations made using PBMCs. However, in contrast to PBMCs, there is also a consistent and dose-dependent induction of IL-12p70-secretion after incubation each agonist compound (FIG. 7j). As observed in PBMCs, dose-response curves for cytokine secretion is highly individual and dependent on the respective responsiveness of the blood donor's immune system.

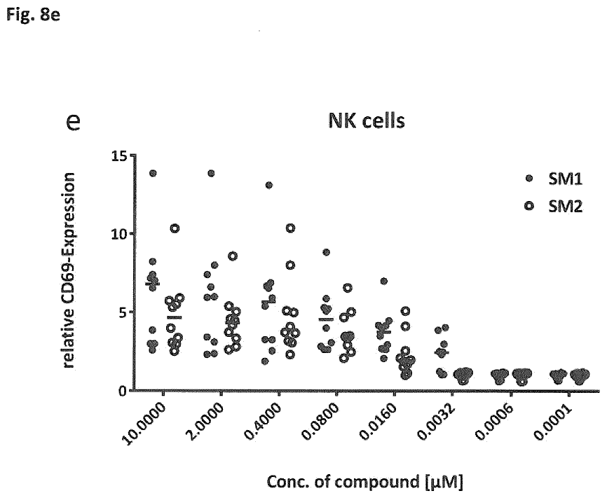

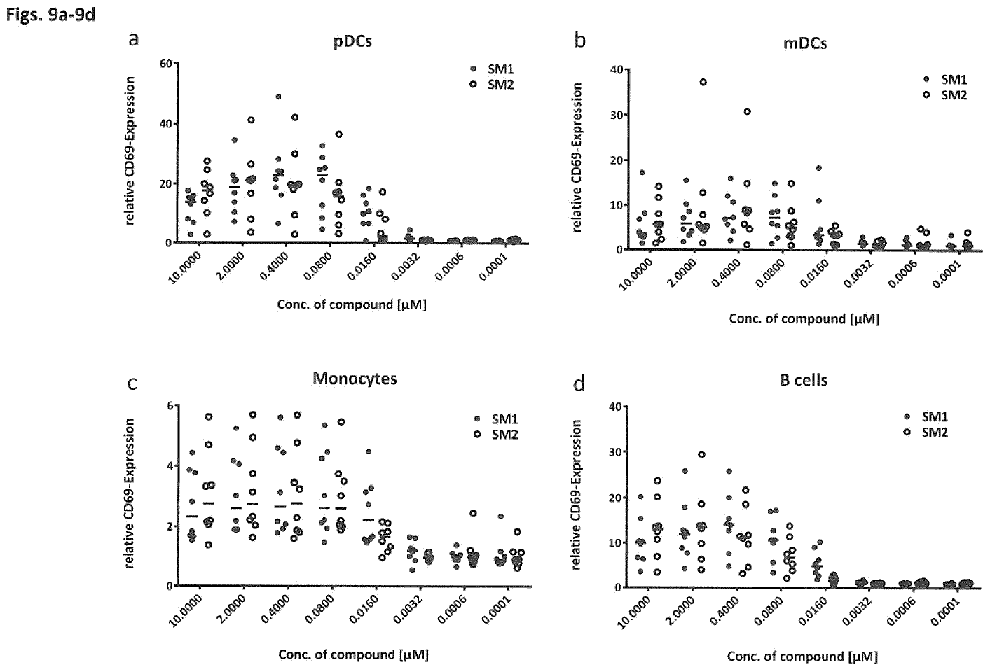

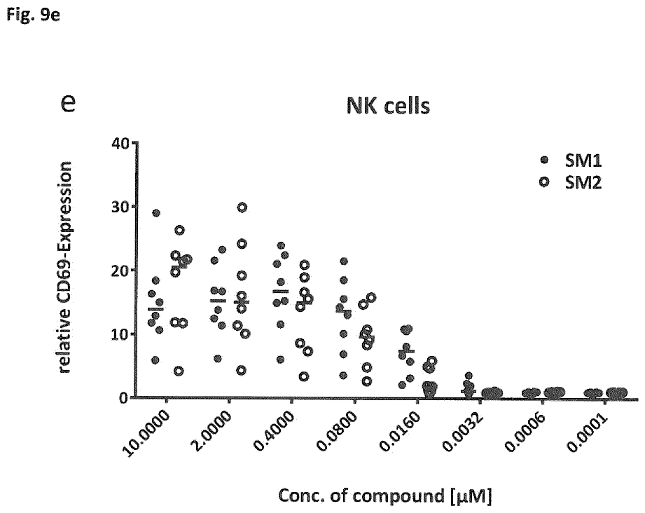

Cellular activation after incubation of PBMCs and whole blood with TLR7-agonists was also analyzed via flow cytometry (expression of CD69). Comparably to the observations made with cytokine secretion, there is a consistent dose-dependent activation of all analyzed immune cell populations (pDCs, mDCs, monocytes, B-cells and NK cells) for both agonist compounds, for PBMCs (FIGS. 8a-8e) and for whole blood (FIGS. 9a-9e). Further, as seen with the cytokines, the intensity of immune cell activation also is highly variable between blood samples from different donors.

The above-described results clearly show that different individuals vary significantly in their intensity of induced cellular immunological reactions in response to an immunotherapeutic agent, in the present case a TLR7-agonist.

### EXAMPLE 4

The objective of the following study was to observe the effect of the in vivo administration of different amounts of small molecule agonists of TLR-7, N-(4-(4-amino-2-(2-methoxyethyl)-1H-imidazo[4,5-c]quinolin-1-yl)butyl)-N-(tetrahydro-2H-pyran-4-yl)acetamide (SM1) and N-(4-(4-amino-2-ethyl-1H-imidazo[4,5-c]quinolin-1-yl)butyl)-N-(1,1-dioxidothietan-3-yl)acetamide (SM3), on expression of various cytokines in the blood in a cynomolgus monkey model. The various cytokines whose expression was determined include interferon alpha, interleukin 1 receptor antagonist, interleukins 4, 6, 8, 10, 12, 15, 18, monocyte chemoattractant protein 1, granulocyte-colony stimulating factor, macrophage inflammatory protein 1 beta, tumor necrosis factor alpha, and vascular endothelial growth factor.

A defined single dose of one of the agonist compounds was administered intravenously to cynomolgus monkeys in a thirty minute infusion. At several time points after initiation of the administration, blood samples (0.5 mL for approximately 0.25 mL plasma) were collected from the monkeys via the vena cephalica antebrachii or vena saphena blood vessel into K3EDTA tubes. The blood samples were stored on crushed ice prior to centrifugation. Plasma was obtained by centrifugation at 4° C. and approximately 1800 g for 10 minutes and was aliquoted into labeled micro tubes and stored frozen at 70° C. or below. Prior to cytokine determination, the frozen plasma samples were thawed, diluted.

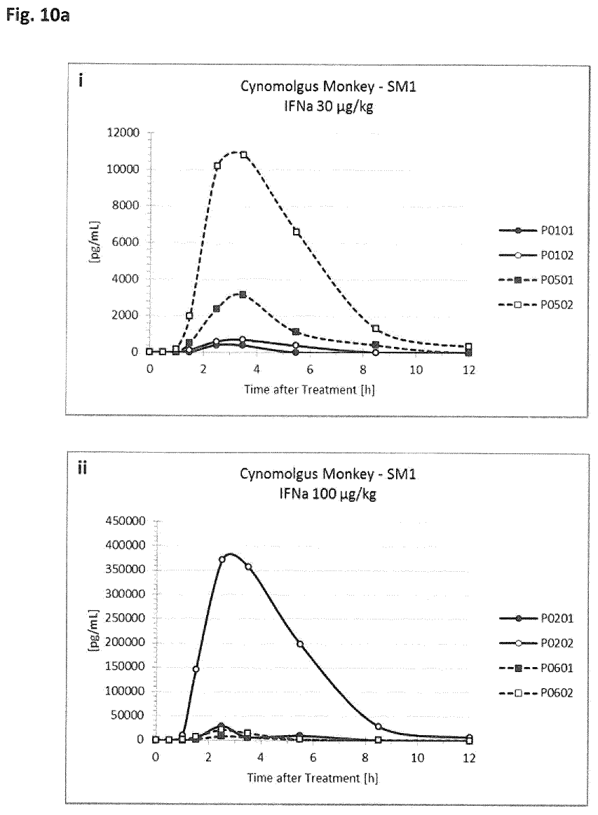

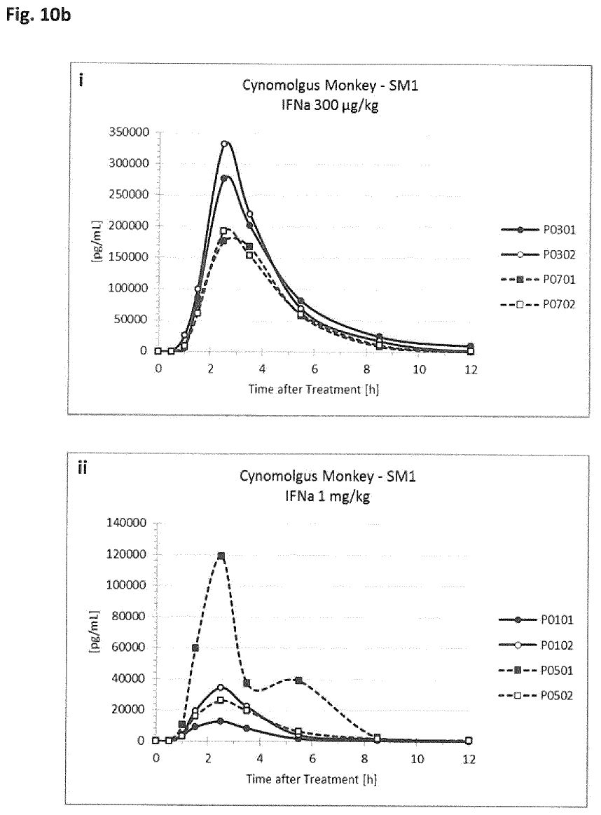

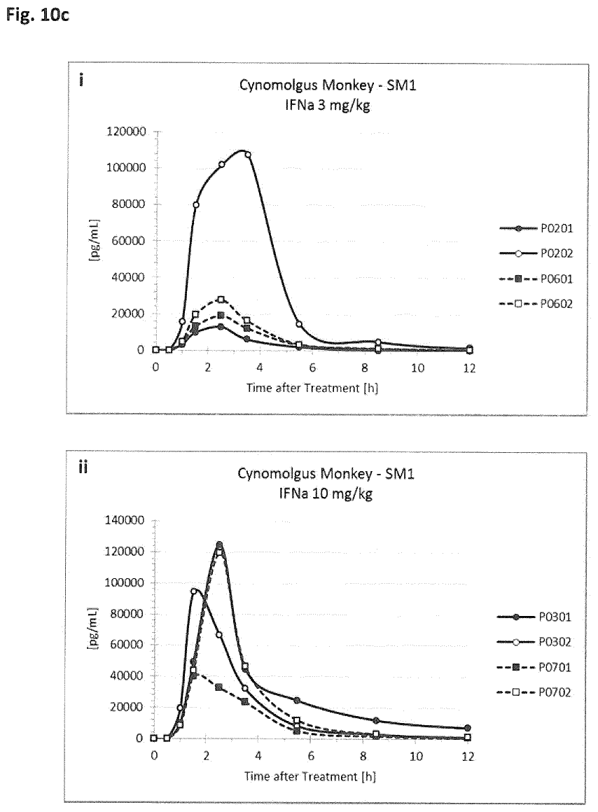

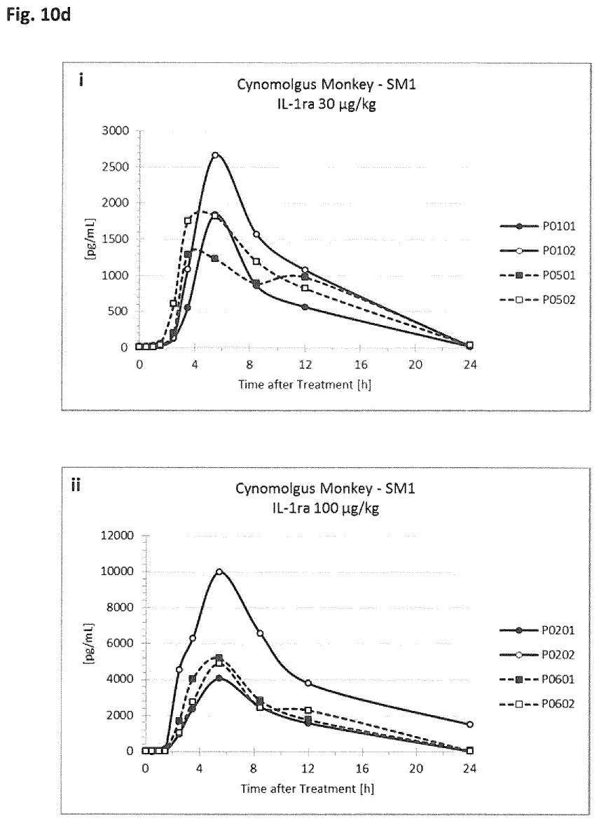

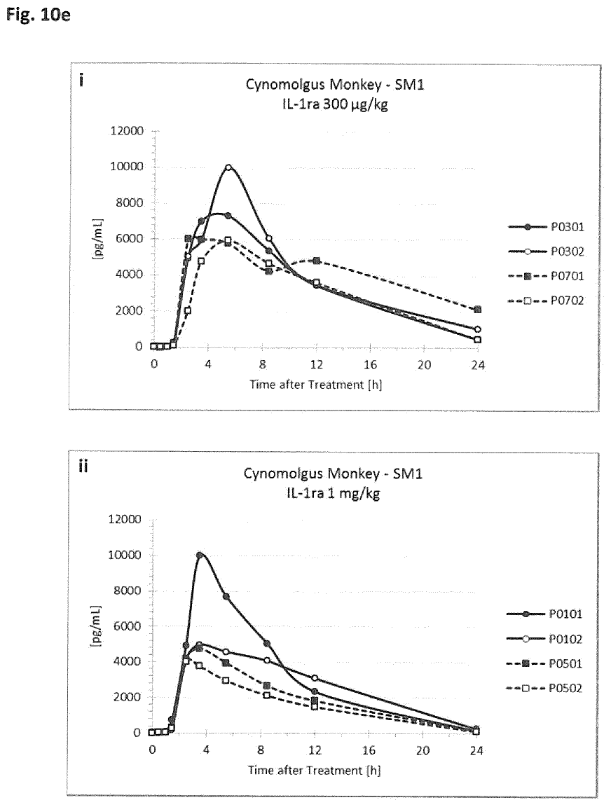

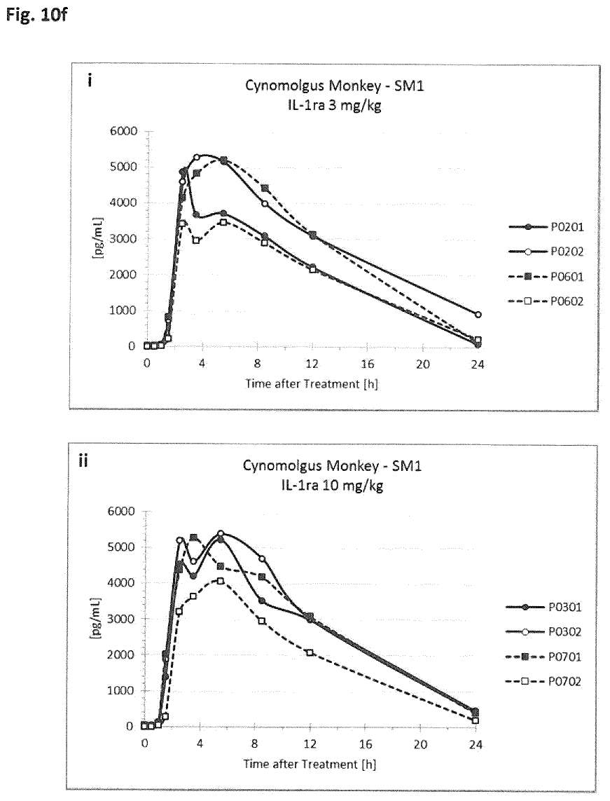

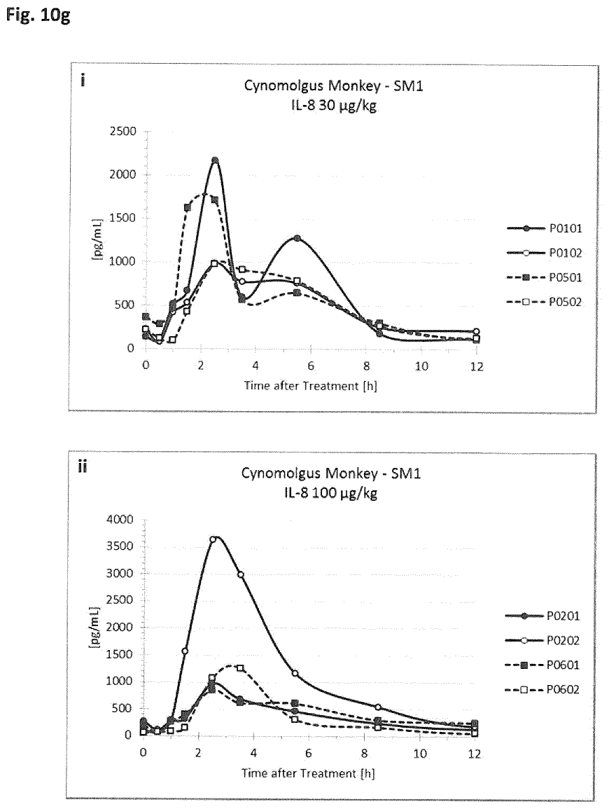

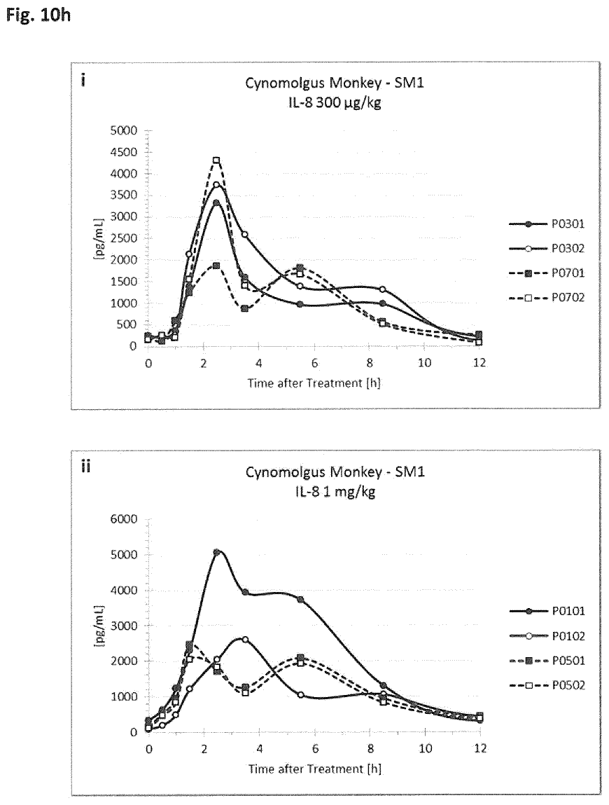

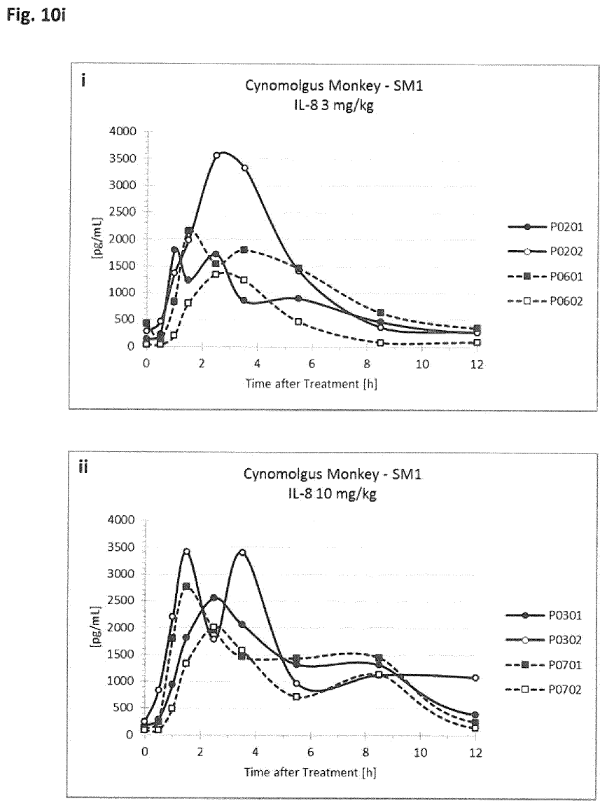

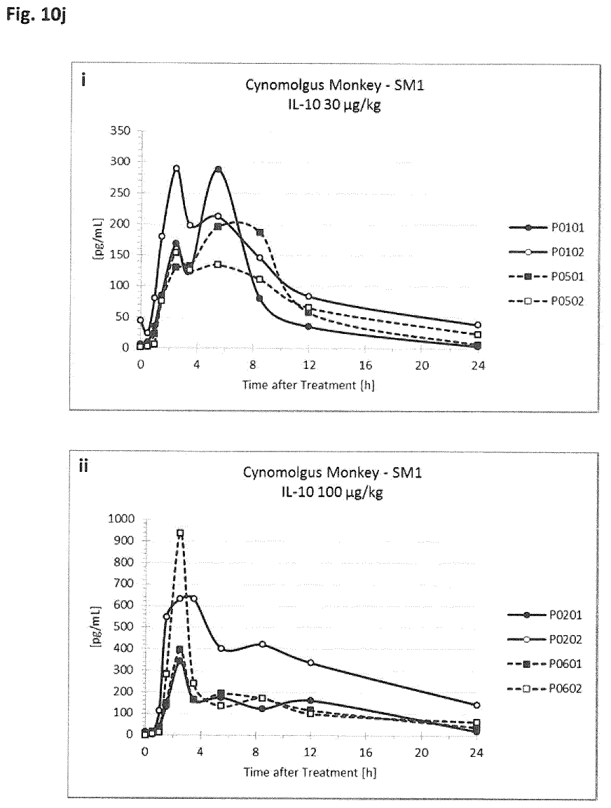

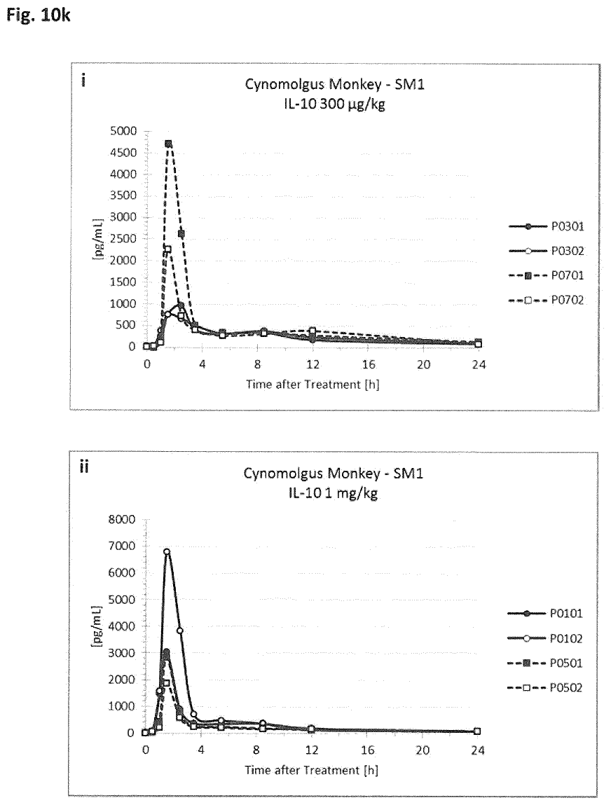

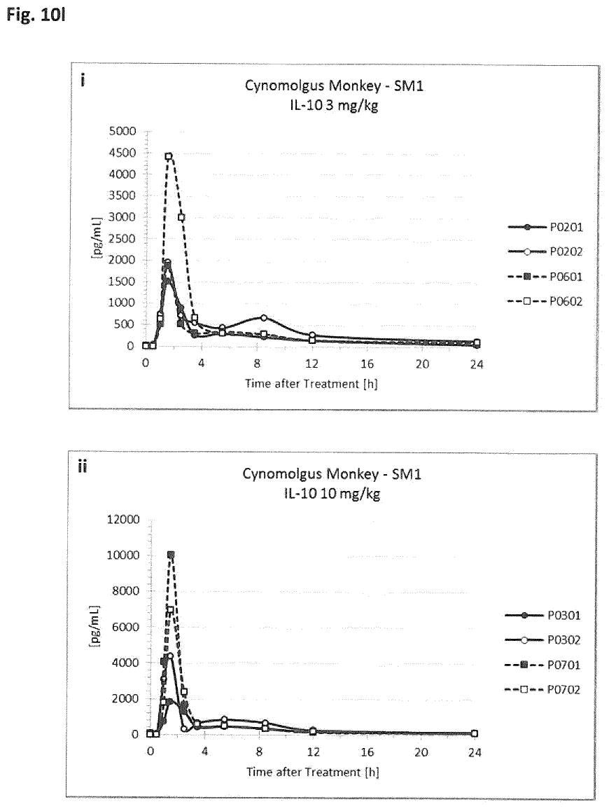

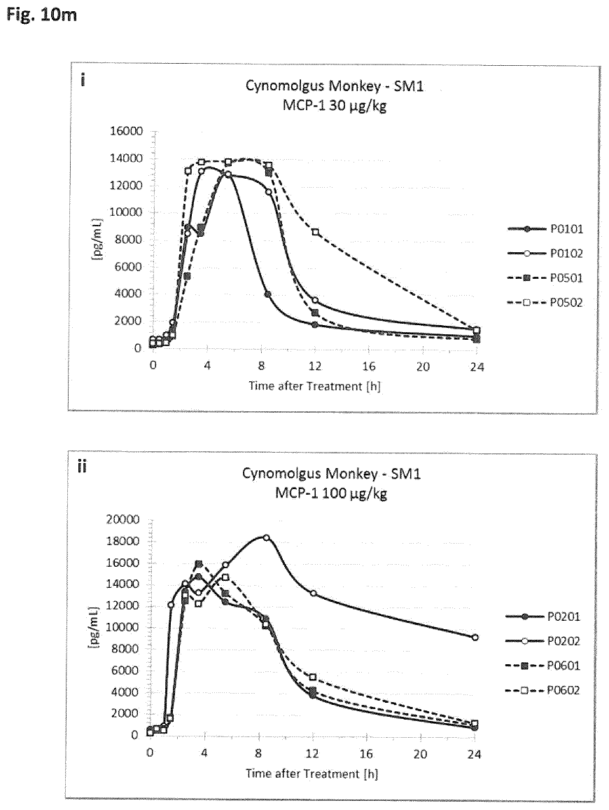

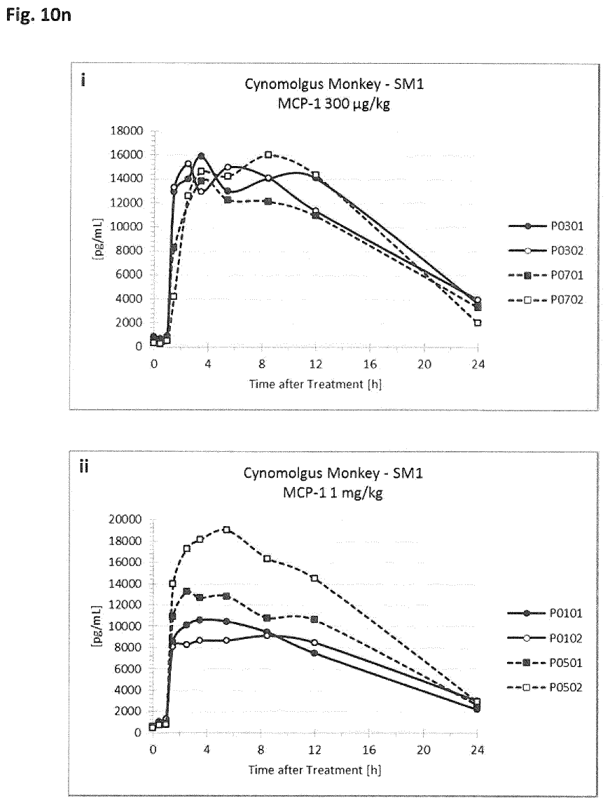

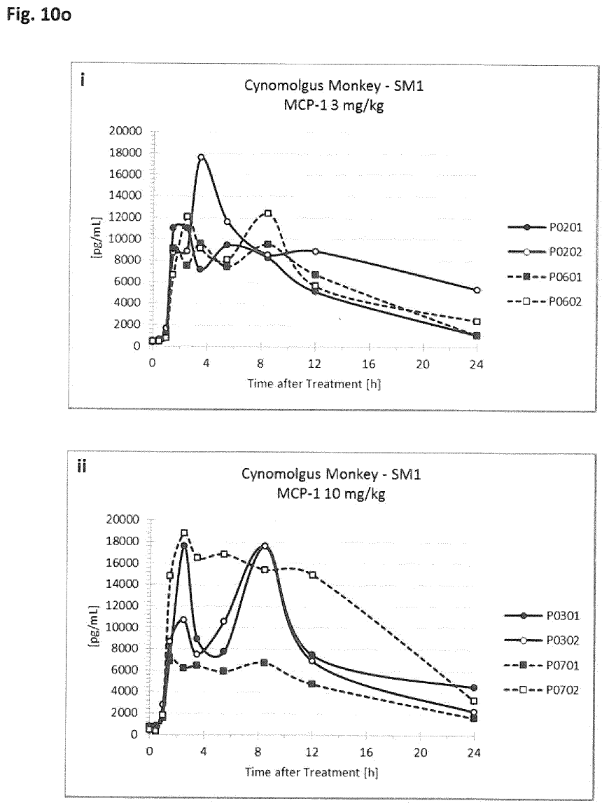

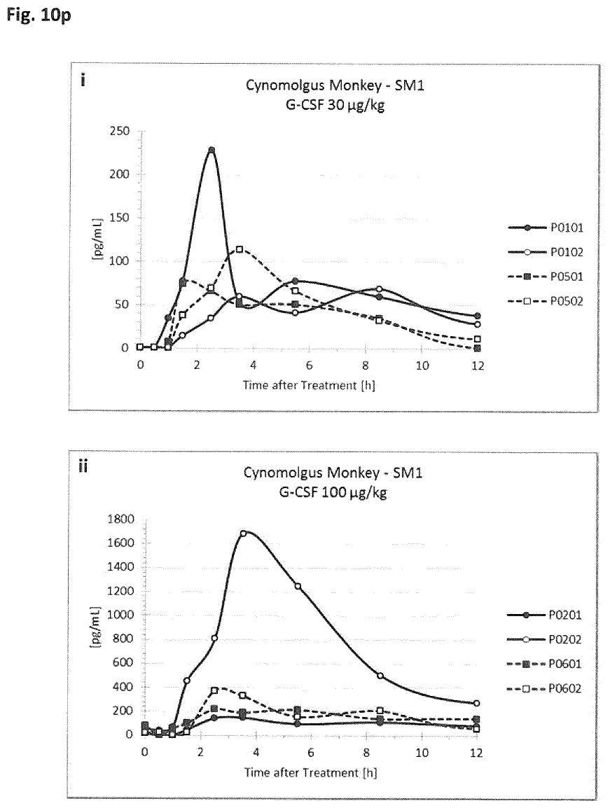

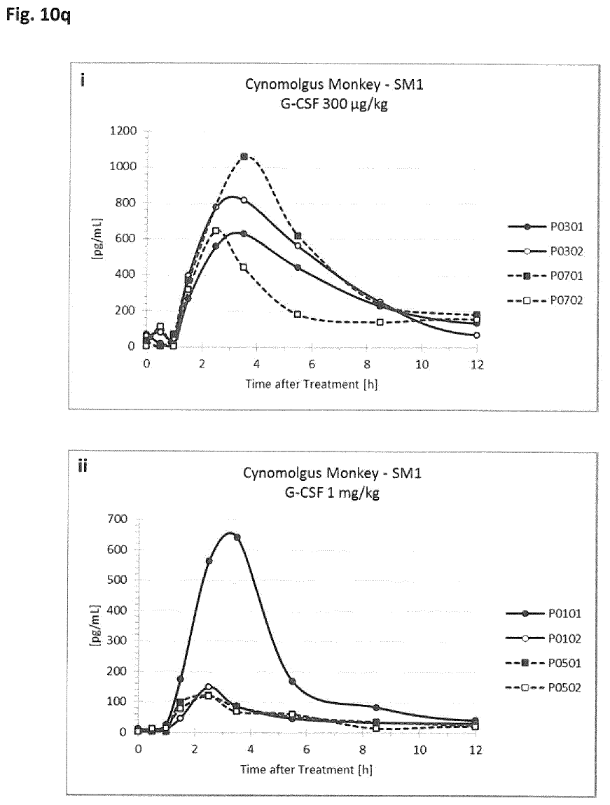

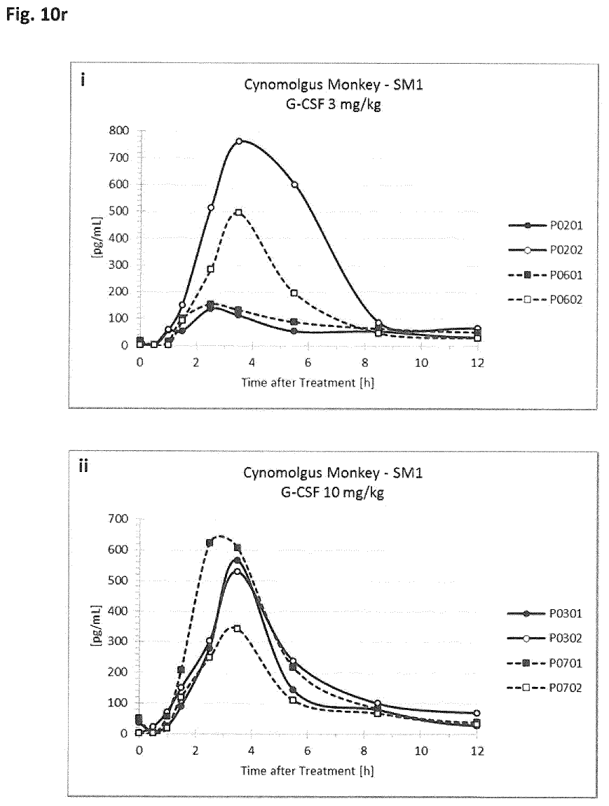

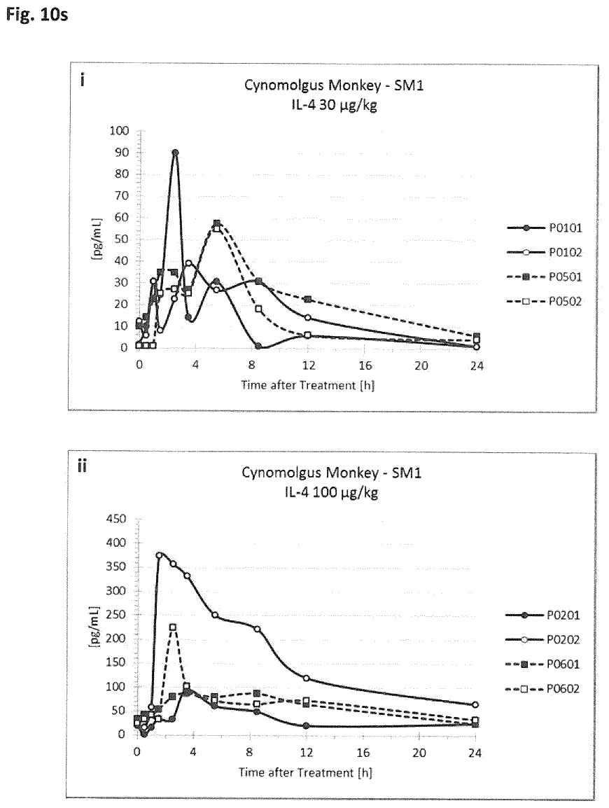

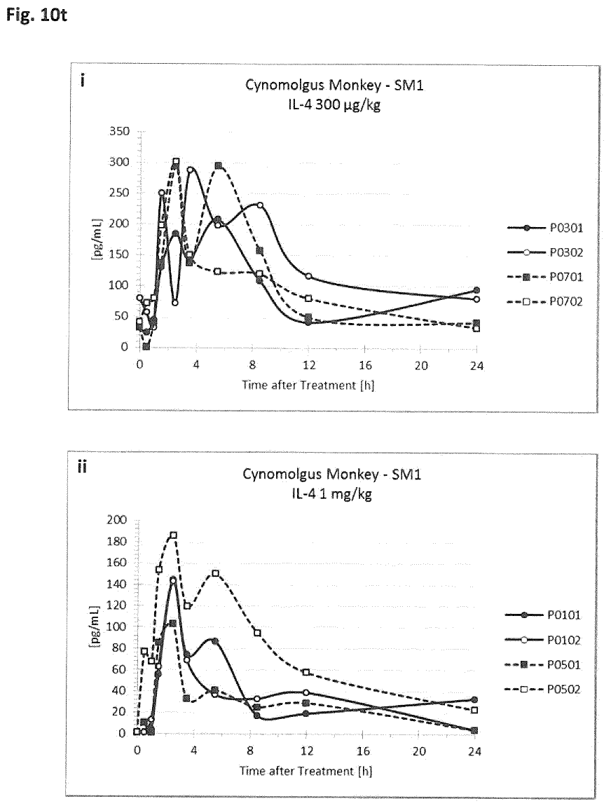

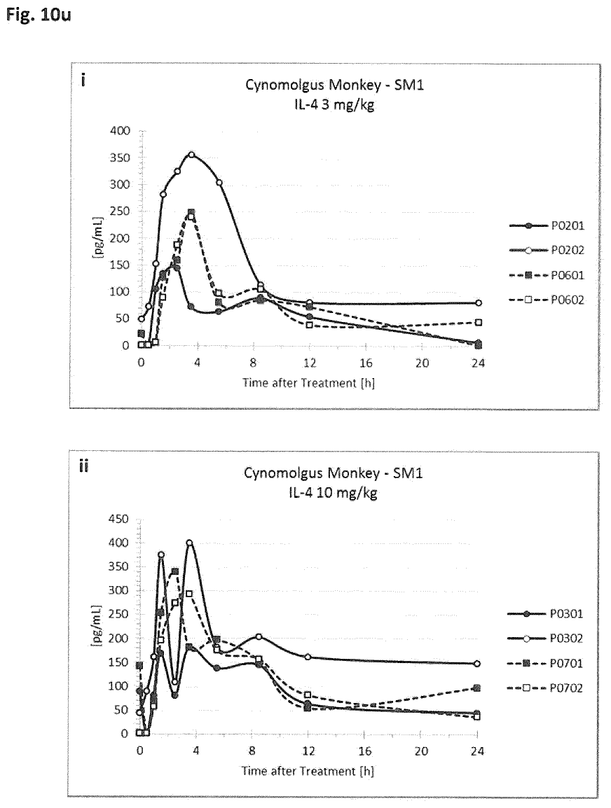

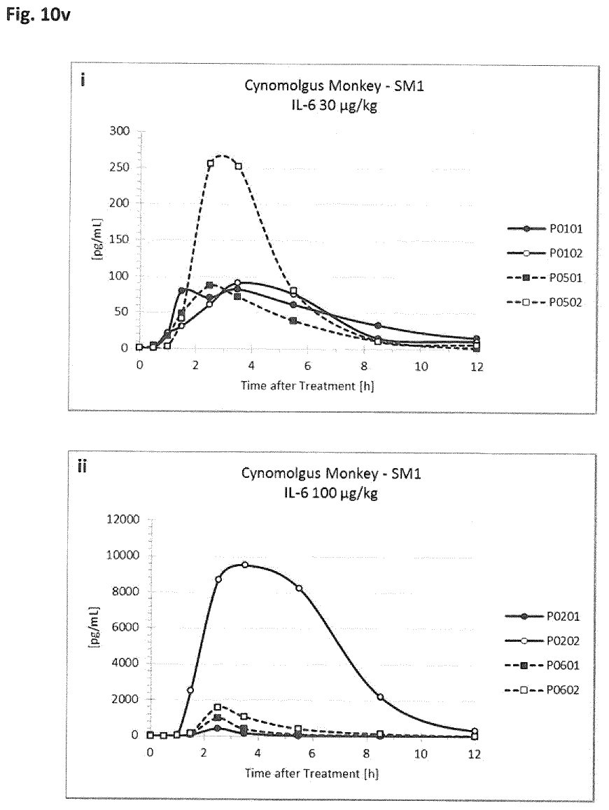

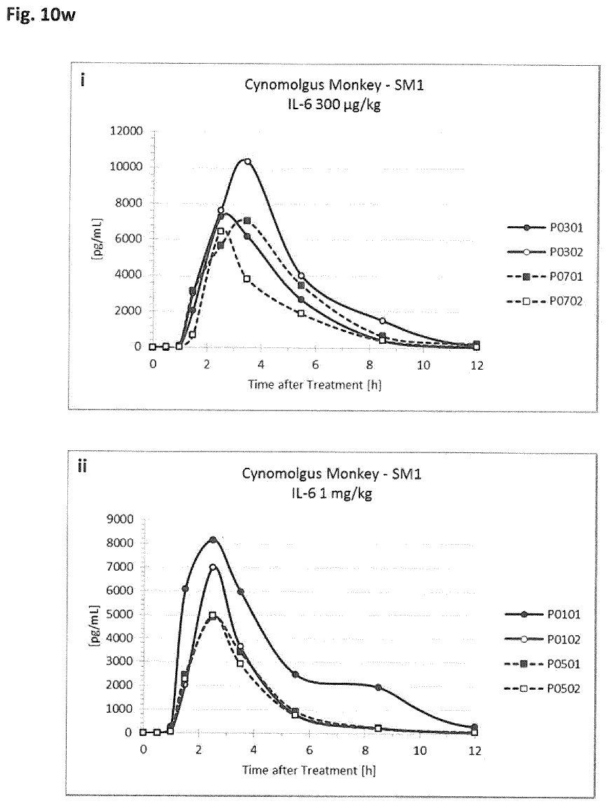

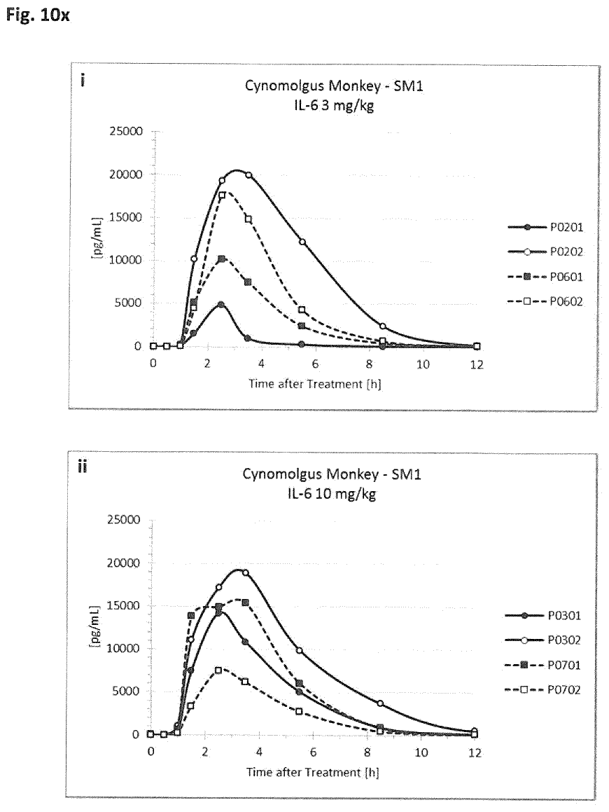

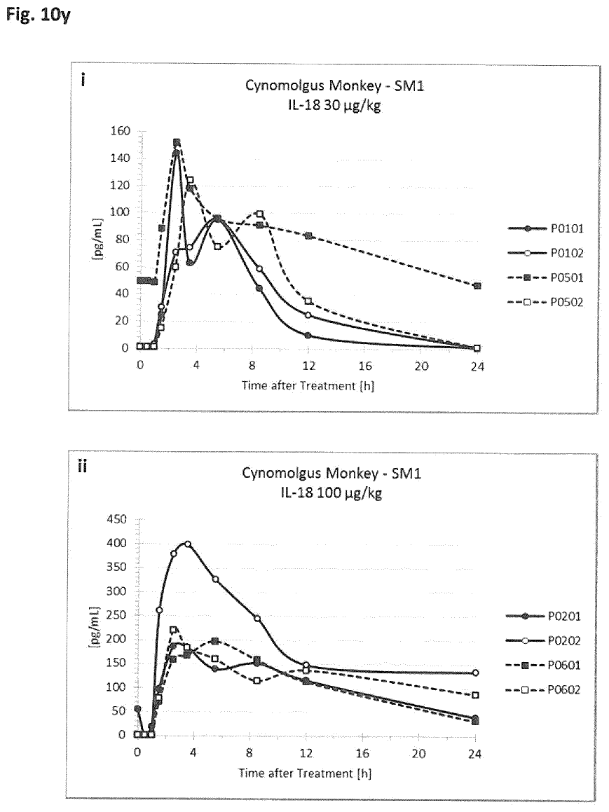

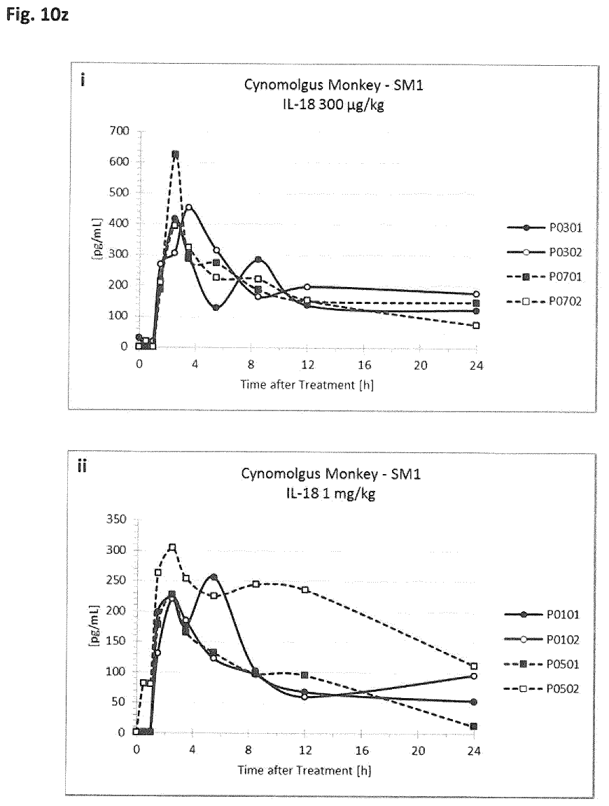

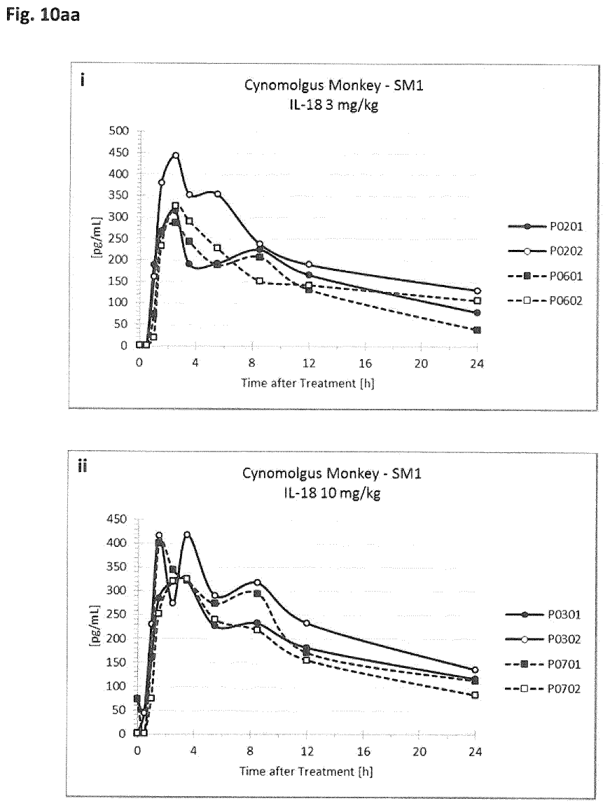

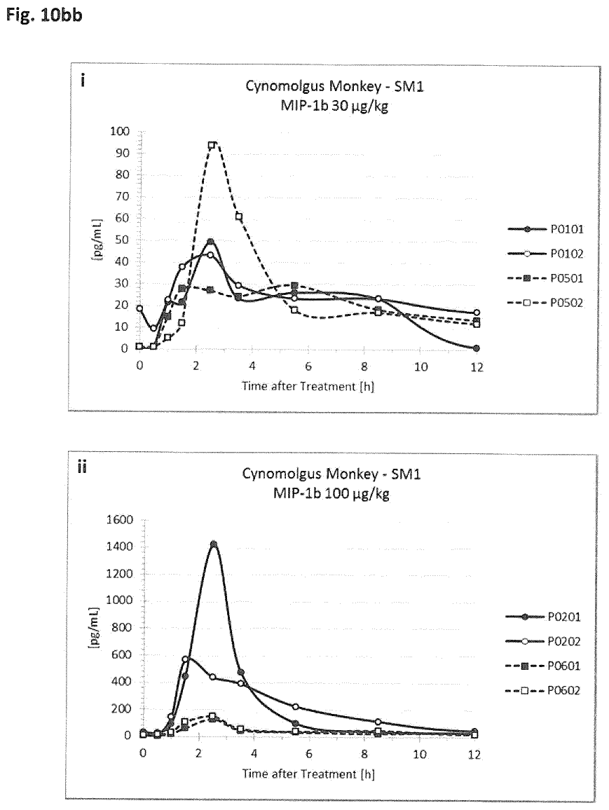

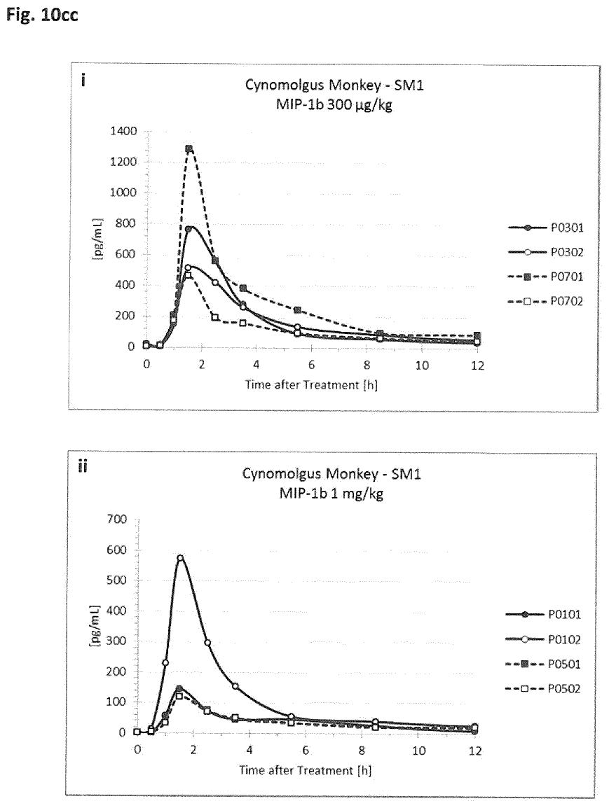

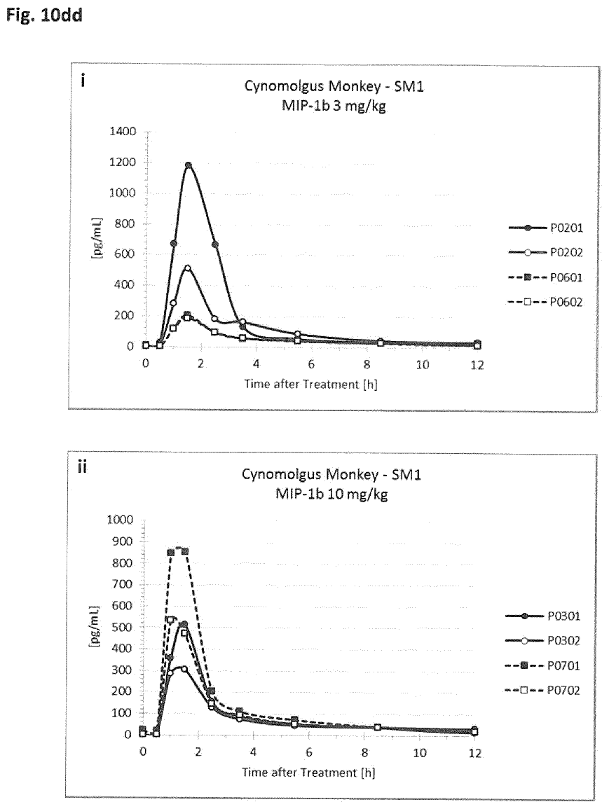

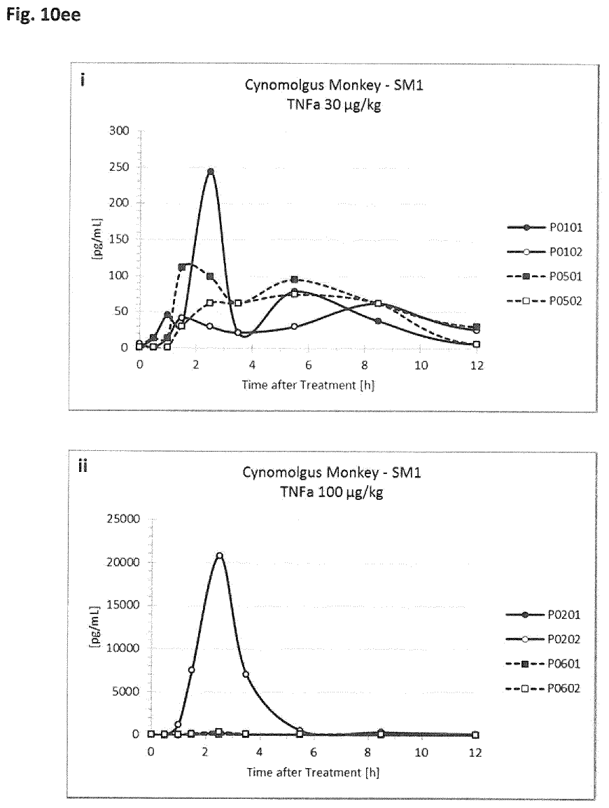

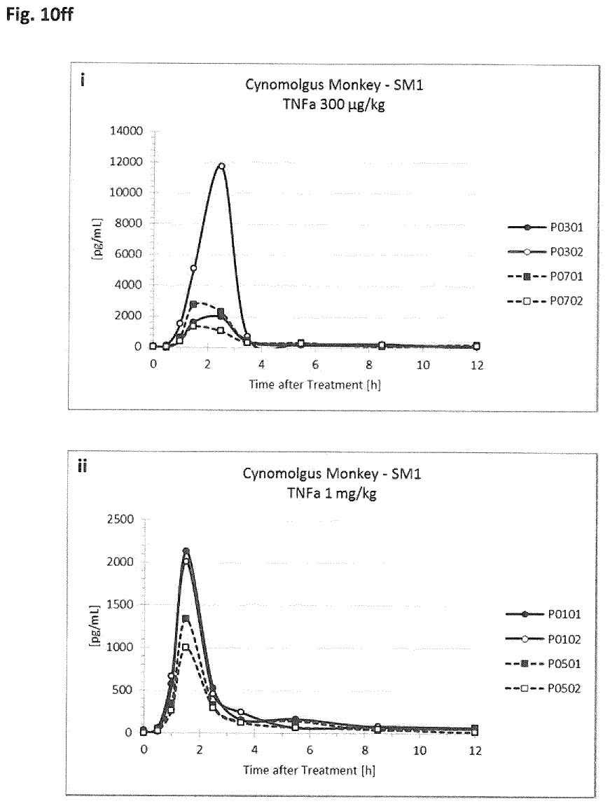

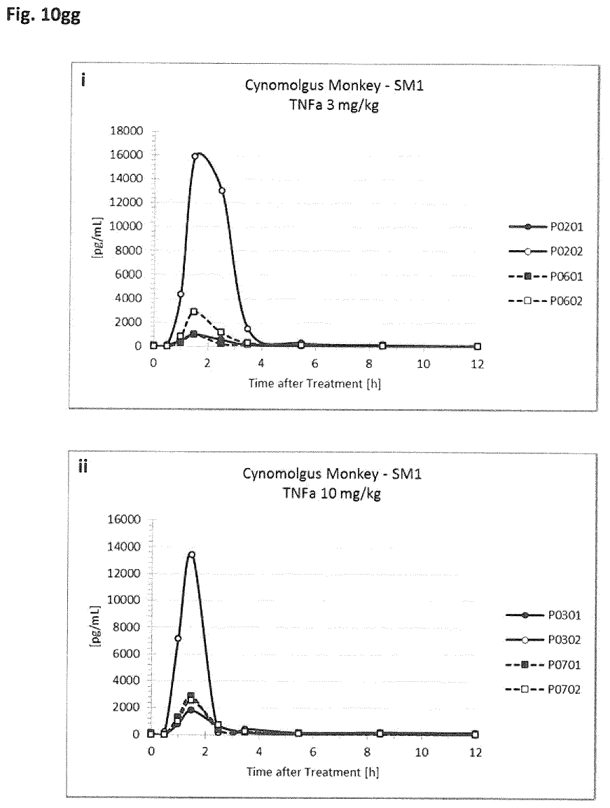

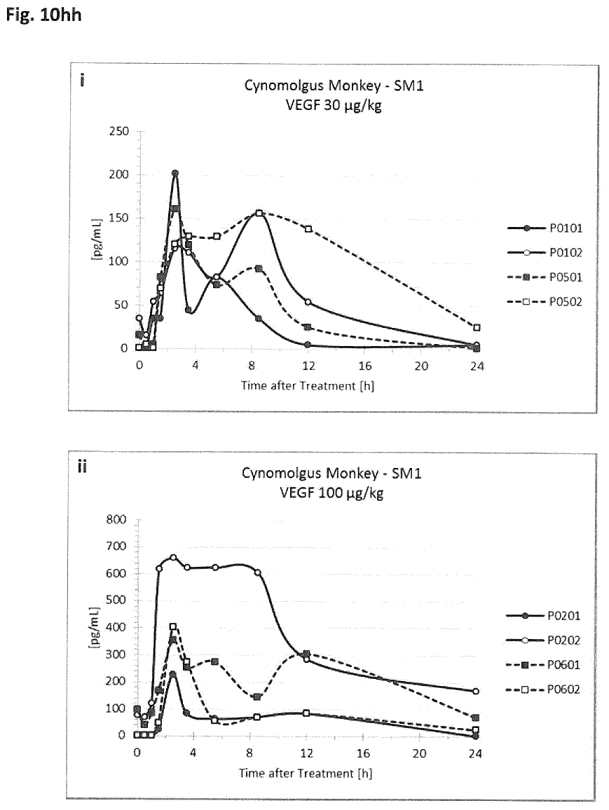

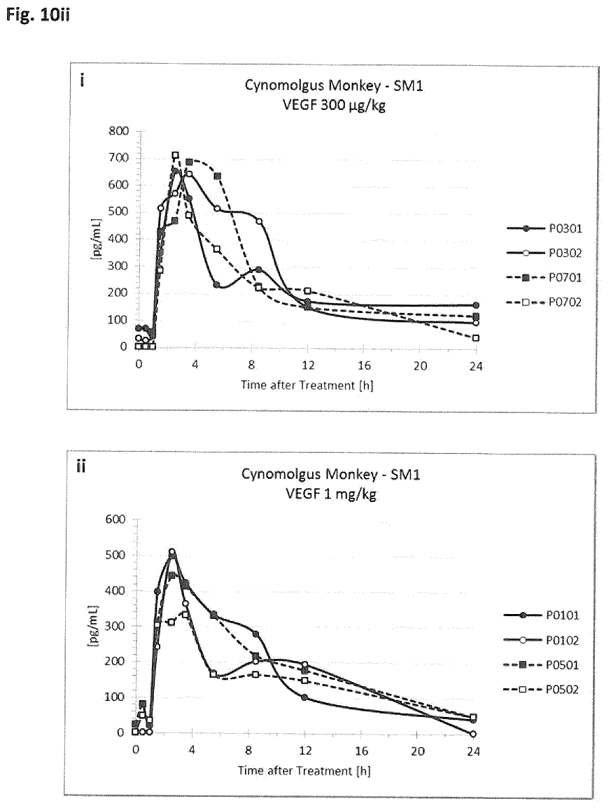

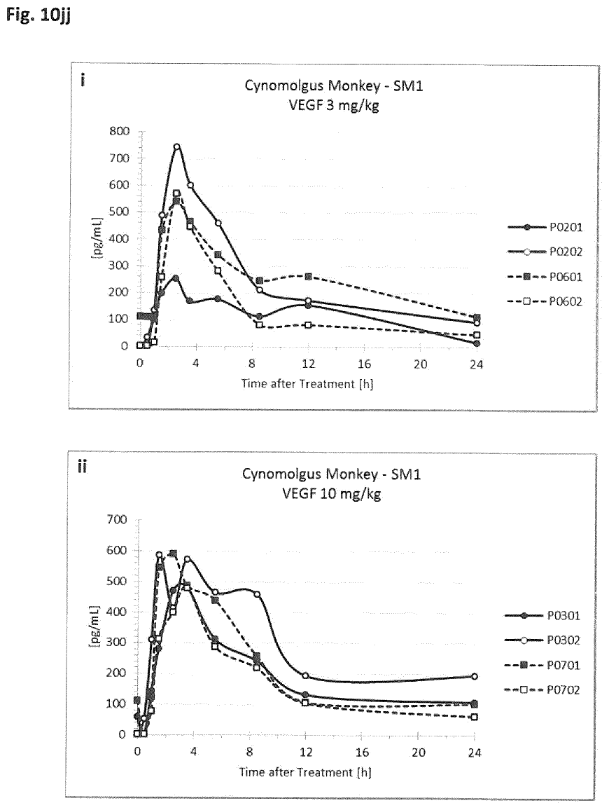

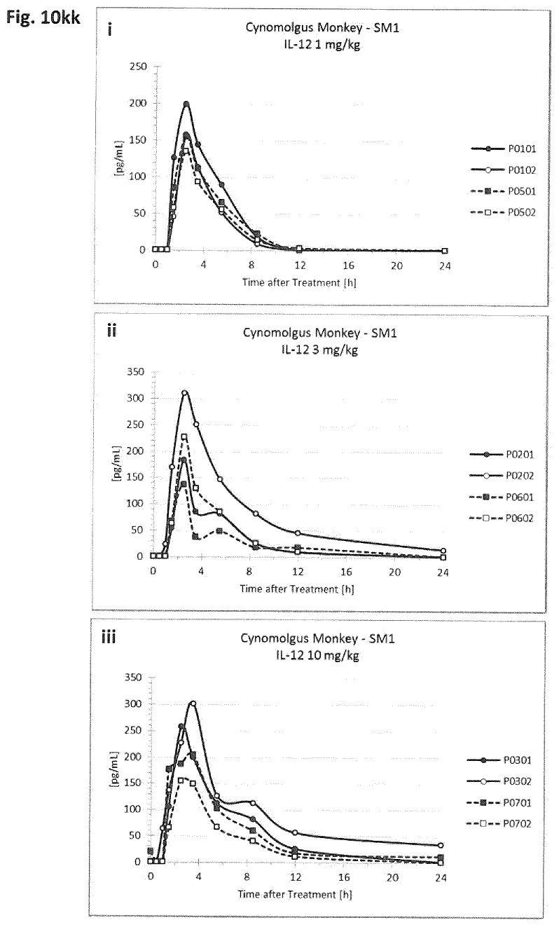

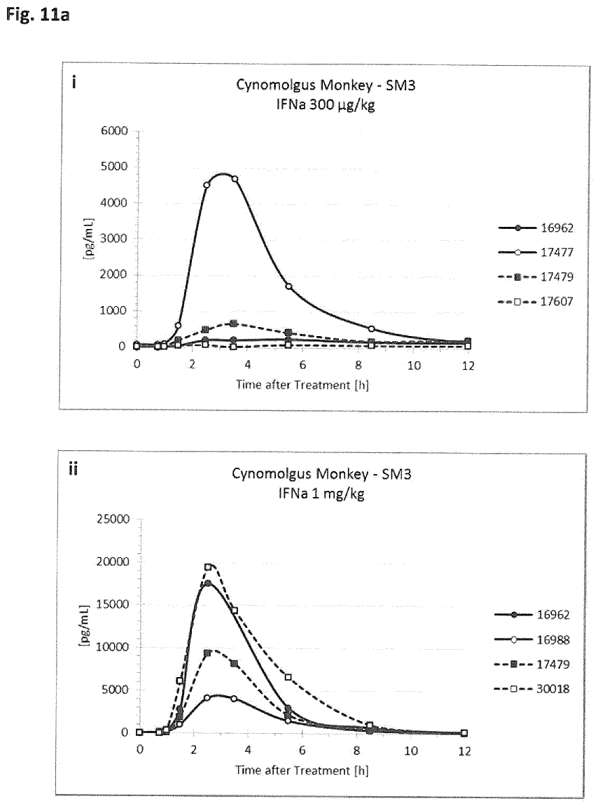

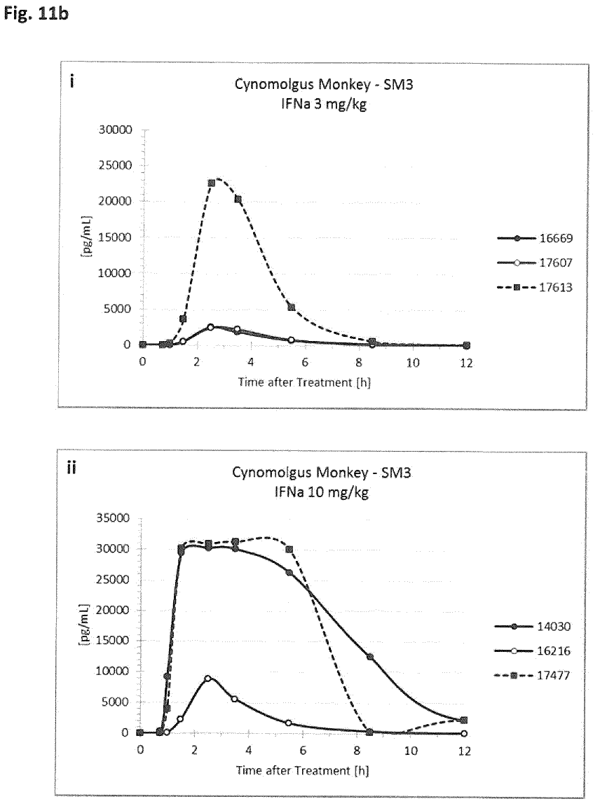

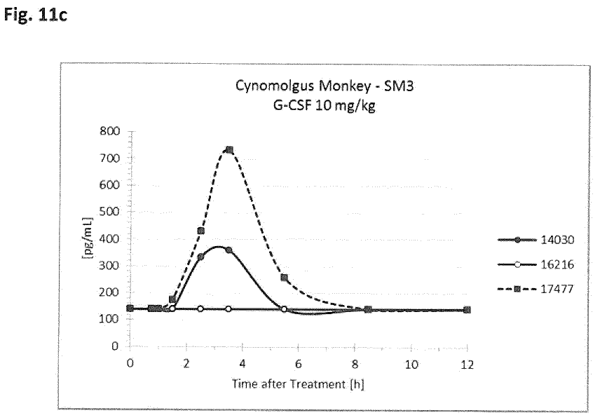

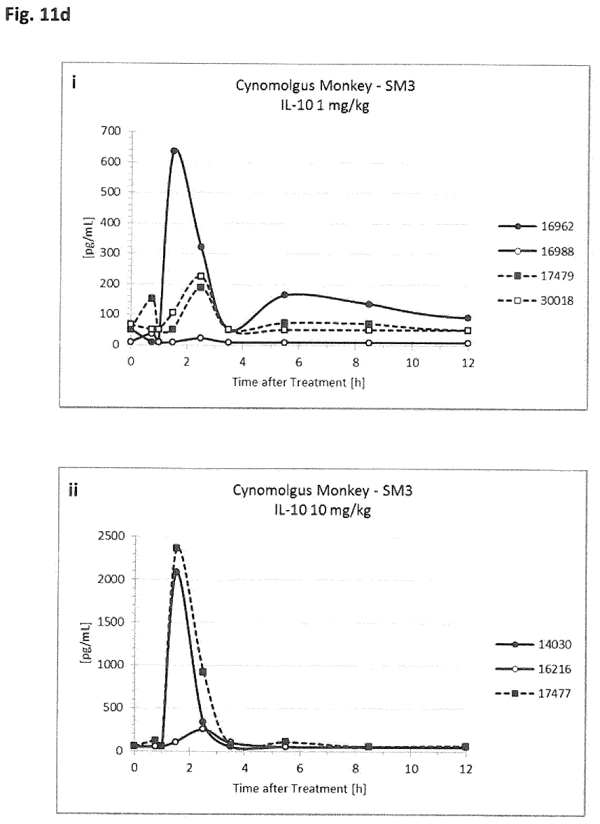

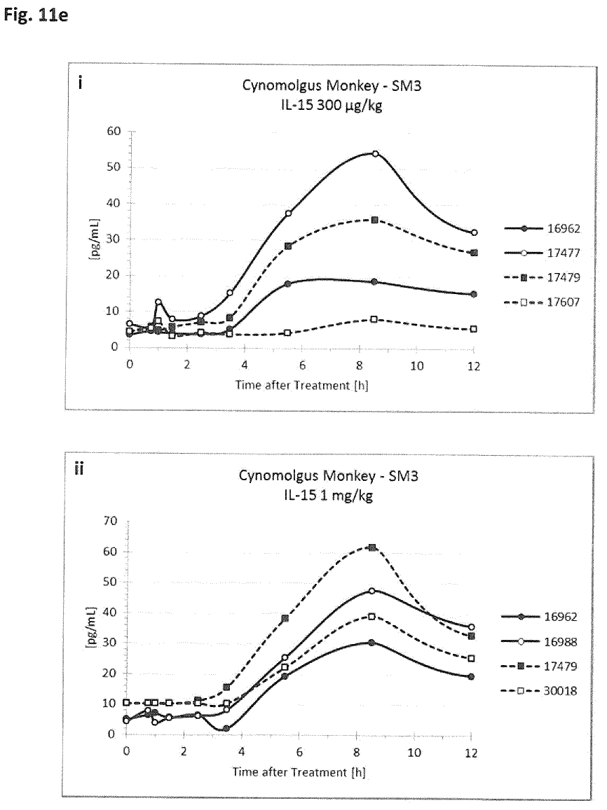

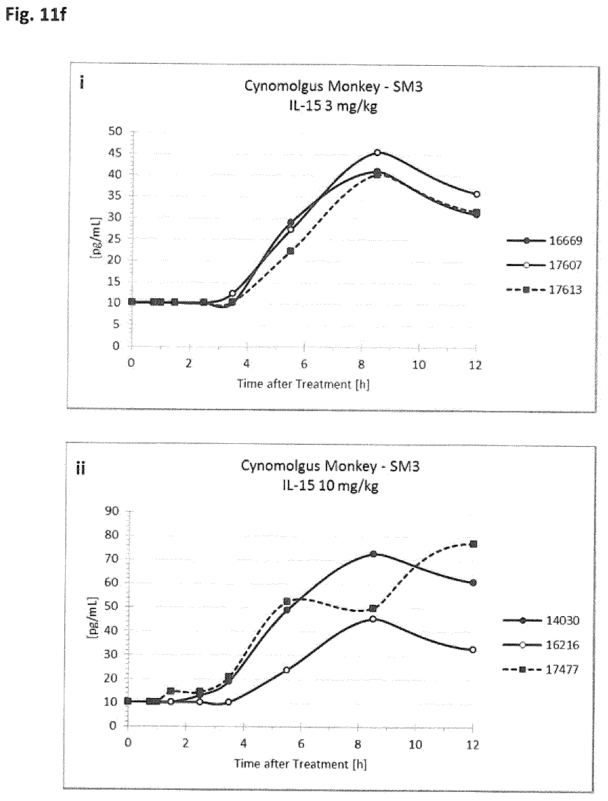

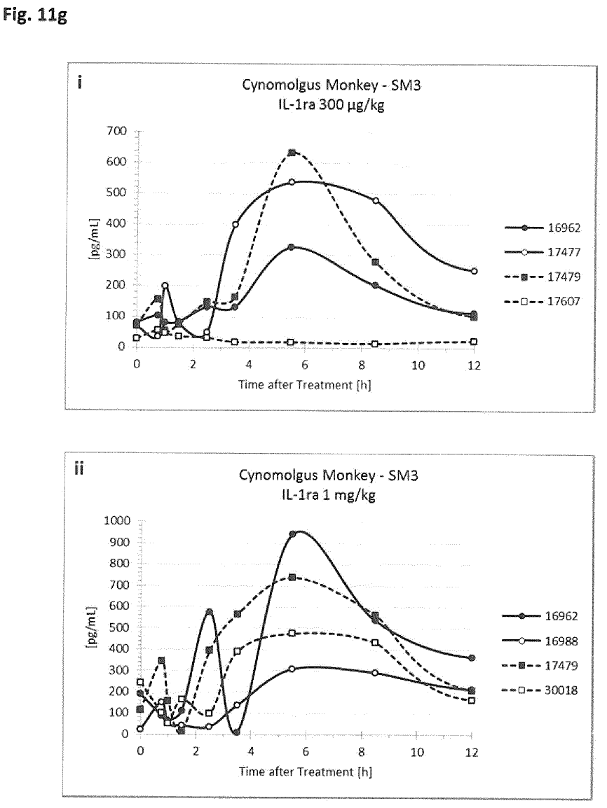

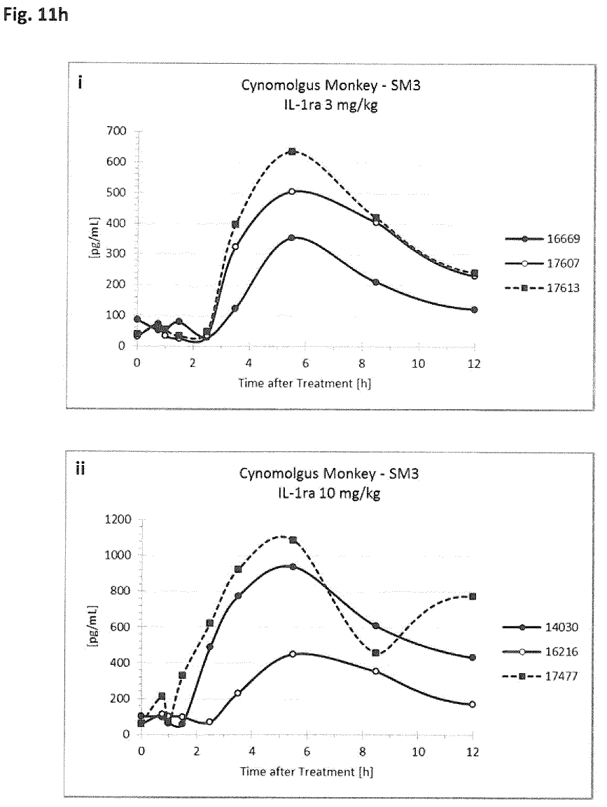

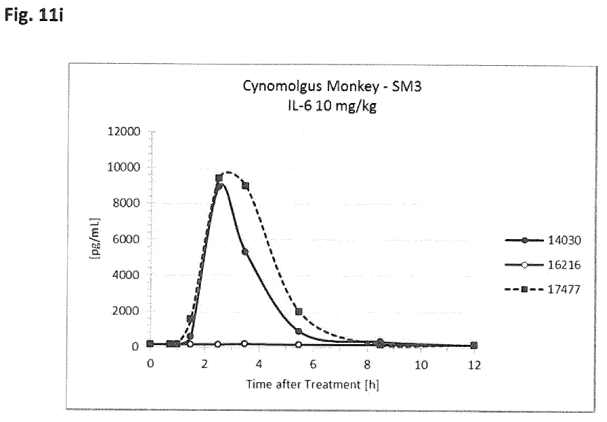

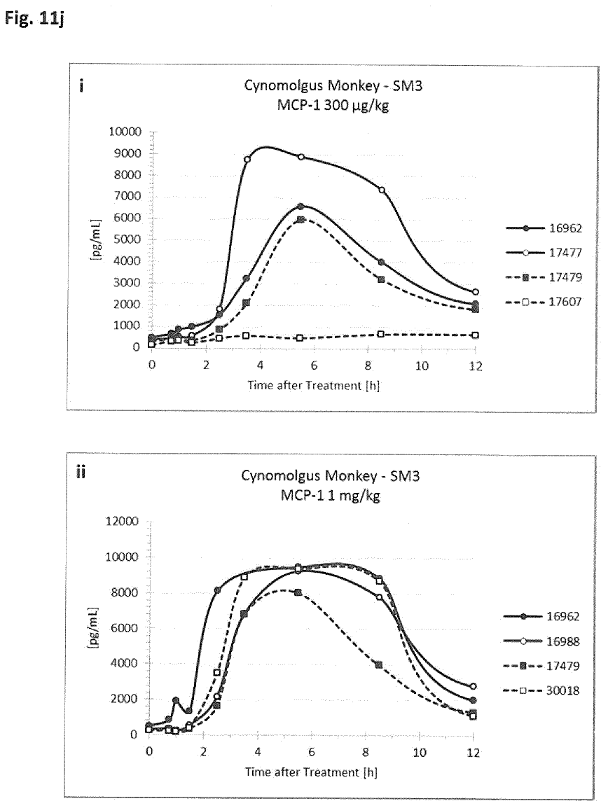

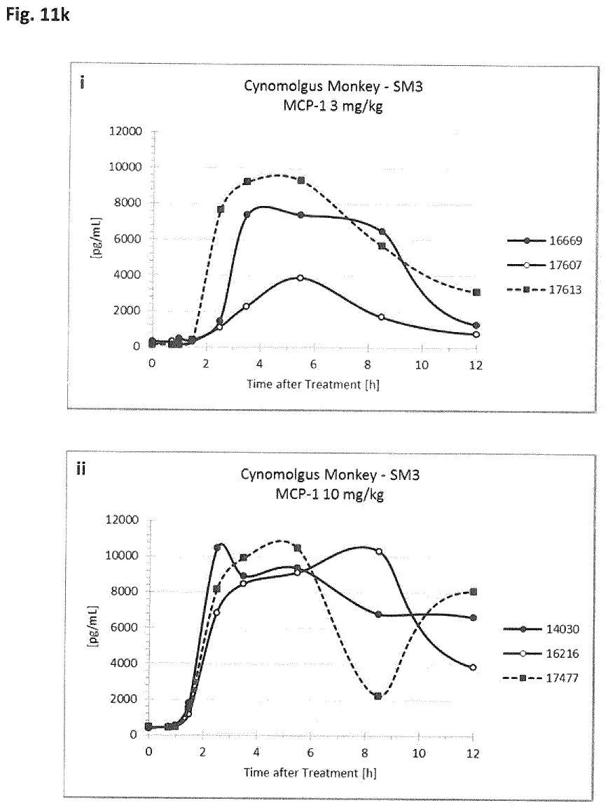

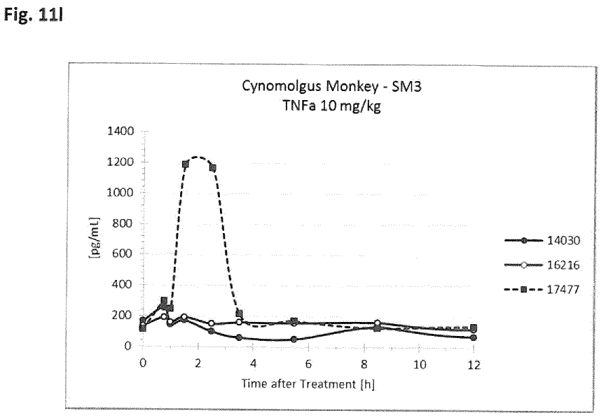

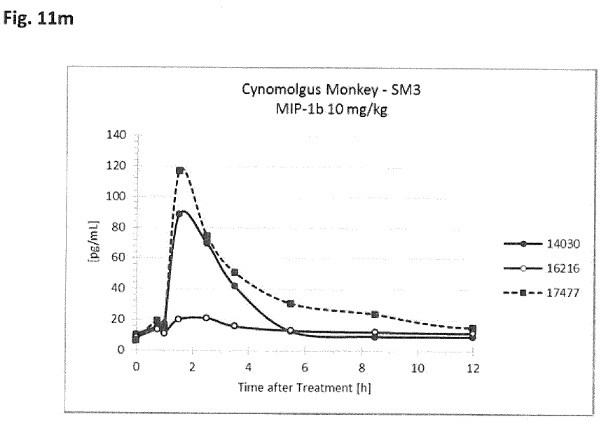

Cytokines levels were determined using an interferon alpha Elisa kit (e.g., VeriKine™ Cynomolgus/Rhesus IFNα ELISA Kit) and a Luminex® non-human primate cytokine/chemokine kit (e.g., Milliplex Non-Human Primate Cytokine/Chemokine Magnetic Premixed 23 Plex Panel), in accordance to the manufacturers' instructions. First, a low dose of the agonist compound (30 [only animals receiving SM1], 100 [only animals receiving SM1] or 300 μg/kg) was administered to the monkeys. Later, at 14 days, a second, higher dose of the same agonist compound (1, 3 or 10 mg/kg) was administered to the same monkeys. Results are depicted in FIGS. 10a-10kk (SM1) and in FIGS. 11a-11m (SM3) and show that in vivo administration of TLR-7 agonists results in the production of various cytokines in a highly individual manner.

FIG. 10 (SM1):

Agonist compound SM1 was administered by an intravenous infusion to cynomolgus monkeys denoted as individuals P0101 (male), P0102 (male), P0501 (female), P0502 (female), P0201 (male), P0202 (male), P0601 (female), P0602 (female), P0301 (male), P0302 (female), P0701 (female), P0702 (female) at doses of 30 μg/kg, 100 mg/kg, 300 μg/kg and 1 mg/kg, 3 mg/kg, and 10 mg/kg, as explained below. Cytokine secretion into blood was measured at various time points until 168 hours after administration. The plasma concentration for various cytokines is shown in the figures at up to 12 or 24 hours after starting the infusion.

Each monkey was given the same agonist twice. The first administration was at one of the low doses, 30 μg/kg, 100 μg/kg or 300 pig/kg and the second administration was at one of the higher doses, 1 mg/kg, 3 mg/kg or 10 mg/kg. Monkeys receiving 30 μg/kg as the first dose were given a second dose of 1 mg/kg. Monkeys receiving 100 μg/kg as the first dose were given a second dose of 3 mg/kg. Monkeys receiving 300 μg/kg as the first dose were given a second dose of 10 mg/kg.
FIG. 10a: Interferon alpha secretion at doses of 30 μg/kg for animals P0101, P0102, P0501, P0502 (i) and 100 μg/kg for animals P0201, P0202, P0601, P0602 (ii)
FIG. 10b: Interferon alpha secretion at doses of 300 μg/kg for animals P0301, P0302, P0701, P0702 (i) and 1 mg/kg for animals P0101, P0102, P0501, P0502 (ii)
FIG. 10c Interferon alpha secretion at doses of 3 mg/kg for animals P0201, P0202, P0601, P0602 (i) and 10 mg/kg for animals P0301, P0302, P0701, P0702 (ii)
FIG. 10d: Interleukin 1 receptor agonist secretion at doses of 30 μg/kg for animals P0101, P0102, P0501, P0502 (i) and 100 μg/kg for animals P0201, P0202, P0601, P0602 (ii)
FIG. 10e: Interleukin 1 receptor agonist secretion at doses of 300 μg/kg for animals P0301, P0302, P0701, P0702 (i) and 1 mg/kg for animals P0101, P0102, P0501, P0502 (ii)
FIG. 10f: Interleukin 1 receptor agonist secretion at doses of 3 mg/kg for animals P0201, P0202, P0601, P0602 (i) and 10 mg/kg for animals P0301, P0302, P0701, P0702 (ii)
FIG. 10g: Interleukin 8 secretion at doses of 30 μg/kg for animals P0101, P0102, P0501, P0502 (i) and 100 μg/kg for animals P0201, P0202, P0601, P0602 (ii)
FIG. 10h: Interleukin 8 secretion at doses of 300 μg/kg for animals P0301, P0302, P0701, P0702 (i) and 1 mg/kg for animals P0101, P0102, P0501, P0502 (ii)
FIG. 10i: Interleukin 8 secretion at doses of 3 mg/kg for animals P0201, P0202, P0601, P0602 (i) and 10 mg/kg for animals P0301, P0302, P0701, P0702 (ii)
FIG. 10j: Interleukin 10 secretion at doses of 30 μg/kg for animals P0101, P0102, P0501, P0502 (i) and 100 μg/kg for animals P0201, P0202, P0601, P0602 (ii)
FIG. 10k: Interleukin 10 secretion at doses of 300 μg/kg for animals P0301, P0302, P0701, P0702 (i) and 1 mg/kg for animals P0101, P0102, P0501, P0502 (ii)
FIG. 10l: Interleukin 10 secretion at doses of 3 mg/kg for animals P0201, P0202, P0601, P0602 (i) and 10 mg/kg for animals P0301, P0302, P0701, P0702 (ii)
FIG. 10m: Monocyte chemoattractant protein 1 secretion at doses of 30 μg/kg for animals P0101, P0102, P0501, P0502 (i) and 100 μg/kg for animals P0201, P0202, P0601, P0602 (ii)
FIG. 10n: Monocyte chemoattractant protein 1 secretion at doses of 300 μg/kg for animals P0301, P0302, P0701, P0702 (i) and 1 mg/kg for animals P0101, P0102, P0501, P0502 (ii)
FIG. 10o: Monocyte chemoattractant protein 1 secretion at doses of 3 mg/kg for animals P0201, P0202, P0601, P0602 (i) and 10 mg/kg for animals P0301, P0302, P0701, P0702 (ii)
FIG. 10p: Granulocyte-colony stimulating factor secretion at doses of 30 μg/kg for animals P0101, P0102, P0501, P0502 (i) and 100 μg/kg for animals P0201, P0202, P0601, P0602 (ii)
FIG. 10q: Granulocyte-colony stimulating factor secretion at doses of 300 μg/kg for animals P0301, P0302, P0701, P0702 (i) and 1 mg/kg for animals P0101, P0102, P0501, P0502 (ii)

FIG. 10r: Granulocyte-colony stimulating factor secretion at doses of 3 mg/kg for animals P0201, P0202, P0601, P0602 (i) and 10 mg/kg for animals P0301, P0302, P0701, P0702 (ii)
FIG. 10s: Interleukin 4 secretion at doses of 30 μg/kg for animals P0101, P0102, P0501, P0502 (i) and 100 μg/kg for animals P0201, P0202, P0601, P0602 (ii)
FIG. 10t: Interleukin 4 secretion at doses of 300 μg/kg for animals P0301, P0302, P0701, P0702 (i) and 1 mg/kg for animals P0101, P0102, P0501, P0502 (ii)
FIG. 10u: Interleukin 4 secretion at doses of 3 mg/kg for animals P0201, P0202, P0601, P0602 (i) and 10 mg/kg for animals P0301, P0302, P0701, P0702 (ii)
FIG. 10v: Interleukin 6 secretion at doses of 30 μg/kg for animals P0101, P0102, P0501, P0502 (i) and 100 μg/kg for animals P0201, P0202, P0601, P0602 (ii)
FIG. 10w: Interleukin 6 secretion at doses of 300 μg/kg for animals P0301, P0302, P0701, P0702 (i) and 1 mg/kg for animals P0101, P0102, P0501, P0502 (ii)
FIG. 10x: Interleukin 6 secretion at doses of 3 mg/kg for animals P0201, P0202, P0601, P0602 (i) and 10 mg/kg for animals P0301, P0302, P0701, P0702 (ii)
FIG. 10y: Interleukin 18 secretion at doses of 30 μg/kg for animals P0101, P0102, P0501, P0502 (i) and 100 μg/kg for animals P0201, P0202, P0601, P0602 (ii)
FIG. 10z: Interleukin 18 secretion at doses of 300 μg/kg for animals P0301, P0302, P0701, P0702 (i) and 1 mg/kg for animals P0101, P0102, P0501, P0502 (ii)
FIG. 10aa: Interleukin 18 secretion at doses of 3 mg/kg for animals P0201, P0202, P0601, P0602 (i) and 10 mg/kg for animals P0301, P0302, P0701, P0702 (ii)
FIG. 10bb: Macrophage inflammatory protein 1 beta secretion at doses of 30 μg/kg for animals P0101, P0102, P0501, P0502 (i) and 100 μg/kg for animals P0201, P0202, P0601, P0602 (ii)
FIG. 10cc: Macrophage inflammatory protein 1 beta secretion at doses of 300 mg/kg for animals P0301, P0302, P0701, P0702 (i) and 1 mg/kg for animals P0101, P0102, P0501, P0502 (ii)
FIG. 10dd: Macrophage inflammatory protein 1 beta secretion at doses of 3 mg/kg for animals P0201, P0202, P0601, P0602 (i) and 10 mg/kg for animals P0301, P0302, P0701, P0702 (ii)
FIG. 10ee: Tumor necrosis factor alpha secretion at doses of 30 μg/kg for animals P0101, P0102, P0501, P0502 (i) and 100 μg/kg for animals P0201, P0202, P0601, P0602 (ii)

FIG. 10ff: Tumor necrosis factor alpha secretion at doses of 300 μg/kg for animals P0301, P0302, P0701, P0702 (i) and 1 mg/kg for animals P0101, P0102, P0501, P0502 (ii)
FIG. 10gg: Tumor necrosis factor alpha secretion at doses of 3 mg/kg for animals P0201, P0202, P0601, P0602 (i) and 10 mg/kg for animals P0301, P0302, P0701, P0702 (ii)
FIG. 10hh: Vascular endothelial growth factor secretion at doses of 30 μg/kg for animals P0101, P0102, P0501, P0502 (i) and 100 μg/kg for animals P0201, P0202, P0601, P0602 (ii)
FIG. 10ii: Vascular endothelial growth factor secretion at doses of 300 μg/kg for animals P0301, P0302, P0701, P0702 (i) and 1 mg/kg for animals P0101, P0102, P0501, P0502 (ii)
FIG. 10jj: Vascular endothelial growth factor secretion at doses of 3 mg/kg for animals P0201, P0202, P0601, P0602 (i) and 10 mg/kg for animals P0301, P0302, P0701, P0702 (ii)
FIG. 10kk: Interleukin 12 secretion at doses of 1 mg/kg for animals P0101, P0102, P0501, P0502 (i), 3 mg/kg for animals P0201, P0202, P0601, P0602 (ii) and 10 mg/kg for animals P0301, P0302, P0701, P0702 (iii)

FIG. 11 (SM3):

Agonist compound SM3 was administered by an intravenous infusion to male cynomolgus monkeys denoted as individuals 16962, 17477, 17479, 17607, 16988, 30018, 16669, 17613, 14030, 16216 in doses of 300 μg/kg, 1 mg/kg, 3 mg/kg, and 10 mg/kg. Cytokine secretion into blood was measured at various time points until 168 hours after administration. The plasma concentration for various cytokines is shown in the figures at up to 12 or 24 hours after starting the infusion.
FIG. 11a: Interferon alpha secretion at doses of 300 μg/kg for animals 16962, 17477, 17479, 17607 (i) and 1 mg/kg for animals 16962, 16988, 17479, 30018 (ii)
FIG. 11b: Interferon alpha secretion at doses of 3 mg/kg for animals 16669, 17607, 17613 (i) and 10 mg/kg for animals 14030, 16216, 17477 (ii)
FIG. 11c: Granulocyte-colony stimulating factor secretion at a dose of 10 mg/kg for animals 14030, 16216, 17477
FIG. 11d: Interleukin 10 secretion at doses of 1 mg/kg for animals 16962, 16988, 17479, 30018 (i) and 10 mg/kg for animals 14030, 16216, 17477 (ii)
FIG. 11e: Interleukin 15 secretion at doses of 300 μg/kg for animals 16962, 17477, 17479, 17607 (i) and 1 mg/kg for animals 16962, 16988, 17479, 30018 (ii)
FIG. 11f: Interleukin 15 secretion at doses of 3 mg/kg for animals 16669, 17607, 17613 (i) and 10 mg/kg for animals 14030, 16216, 17477 (ii)
FIG. 11g: Interleukin 1 receptor agonist secretion at doses of 300 μg/kg for animals 16962, 17477, 17479, 17607 (i) and 1 mg/kg for animals 16962, 16988, 17479, 30018 (ii)
FIG. 11h: Interleukin 1 receptor agonist secretion at doses of 3 mg/kg for animals 16669, 17607, 17613 (i) and 10 mg/kg for animals 14030, 16216, 17477 (ii)
FIG. 11i: Interleukin 10 secretion at a dose of 10 mg/kg for animals 14030, 16216, 17477
FIG. 11j: Monocyte chemoattractant protein 1 secretion at doses of 300 μg/kg for animals 16962, 17477, 17479, 17607 (i) and 1 mg/kg for animals 16962, 16988, 17479, 30018 (ii)
FIG. 11k: Monocyte chemoattractant protein 1 secretion at doses of 3 mg/kg for animals 16669, 17607, 17613 (i) and 10 mg/kg for animals 14030, 16216, 17477 (ii)
FIG. 11l: Tumor necrosis factor alpha secretion at a dose of 10 mg/kg for animals 14030, 16216, 17477
FIG. 11m: Macrophage inflammatory protein 1 beta secretion at a dose of 10 mg/kg for animals 14030, 16216, 17477.

### EXAMPLE 5

The objective of the following study was to observe the effect of two small molecule agonists of TLR8, 2-ethyl-1-(4-((2-methyltetrahydrofuran-3-yl)amino)butyl)-1H-imidazo[4,5-c]quinolin-4-amine (SM4) and 1-(4-(cyclohexylamino)butyl)-2-ethyl-1H-imidazo[4,5-c]quinolin-4-amine (SM5) on cytokine secretion by human PBMCs in vitro. The various cytokines whose expression was determined include tumor necrosis factor alpha, interleukin 1 beta, interleukin 6, 8, 10 and 12p′70, interferon gamma, interleukin 10, and interferon gamma inducible protein 10.

PBMCs were isolated from fresh blood samples withdrawn from four human voluntary blood donors. The PBMCs were isolated according to standard protocols, resuspended in cell culture medium containing 10% fetal calf serum at a cell count of 2×10⁶/mL, seeded into 96-well plates at 100 μl per well and subsequently incubated for 6 hours at 37° C. Appropriate stock solutions of each agonist compound were produced by dissolving the agonist compound in DMSO and subsequently diluting in one or several steps with DMSO to a concentration of 1000-fold of the final test concentration. Appropriate pre-dilutions of the agonist were prepared with medium; in a first step the agonist was diluted 1:100, and in a second step 25 μL of pre-dilution and 125 μL of medium were added to 1004, of cells in the wells. The cells were incubated for 24 hours at 37° C. and supernatants were then harvested and analyzed with a Luminex® bead assay or an ELISA assay specific for the particular human cytokines according to the manufacturers' instructions.

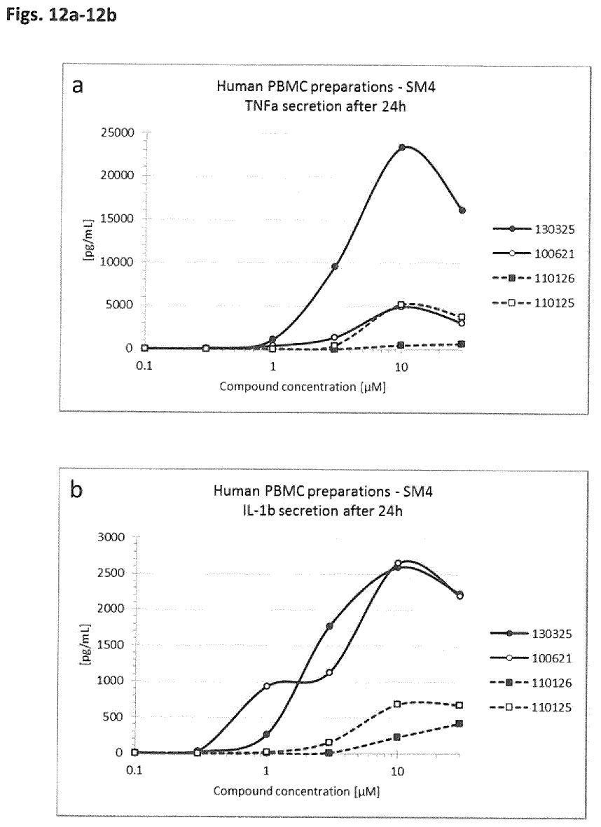

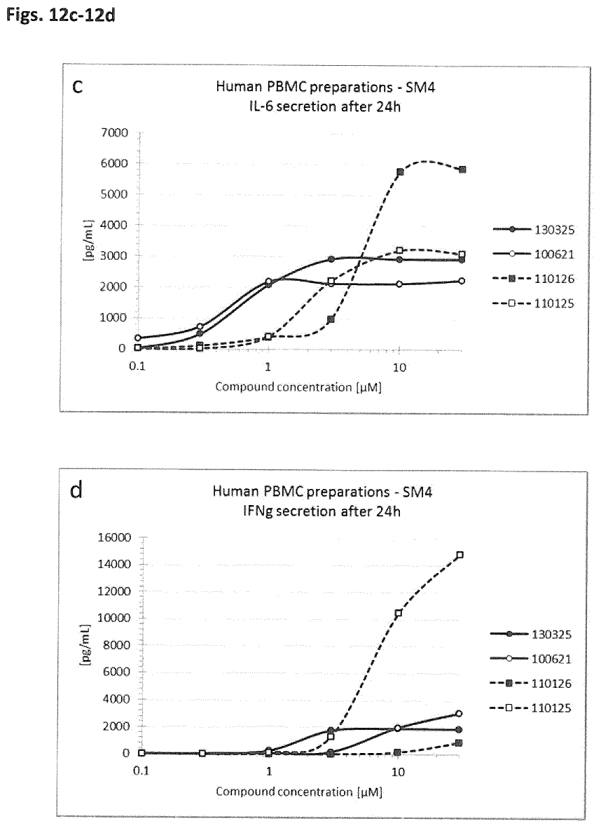

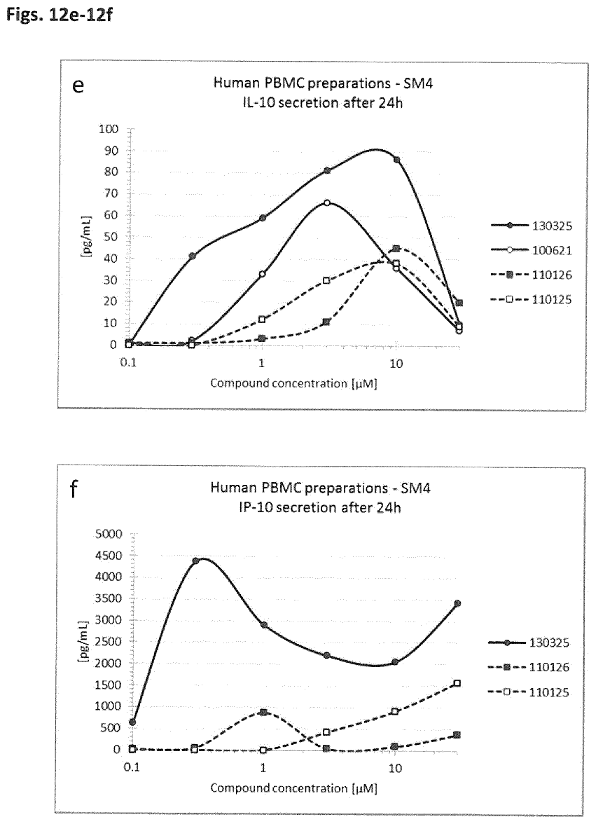

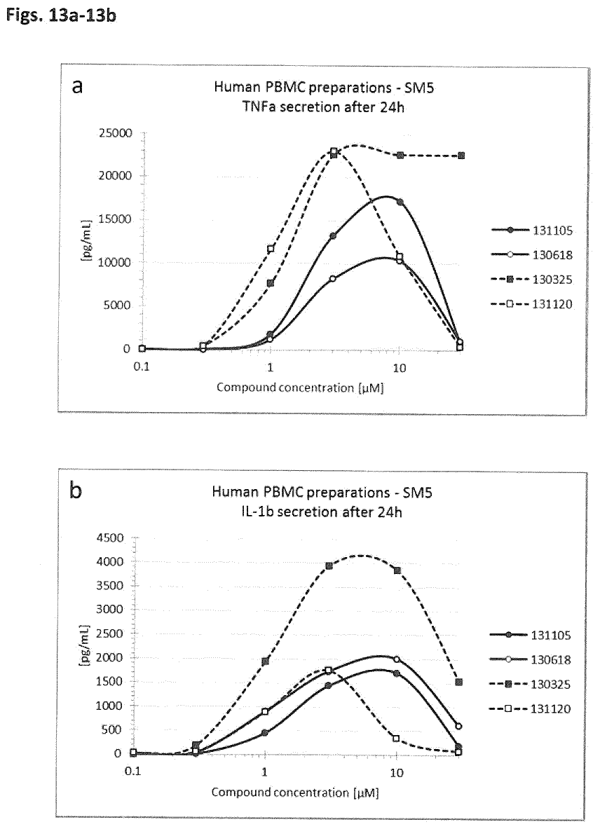

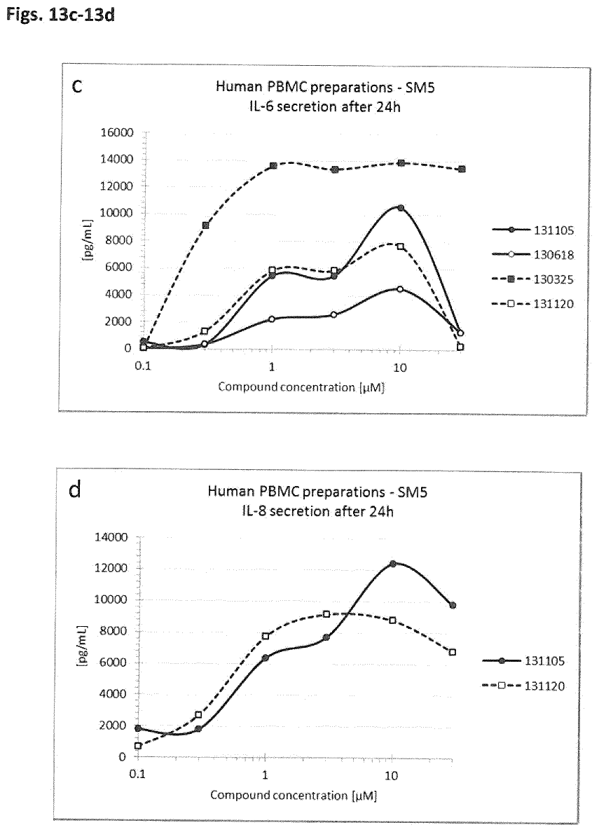

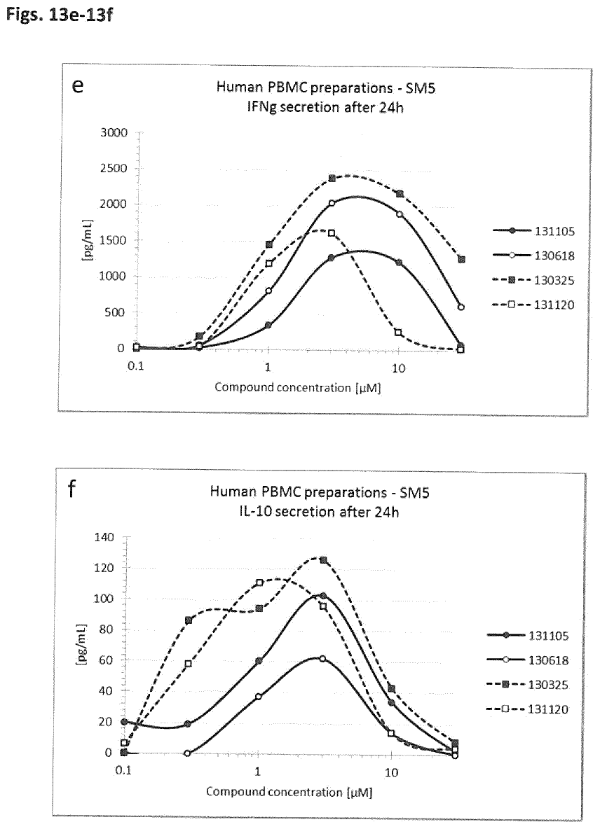

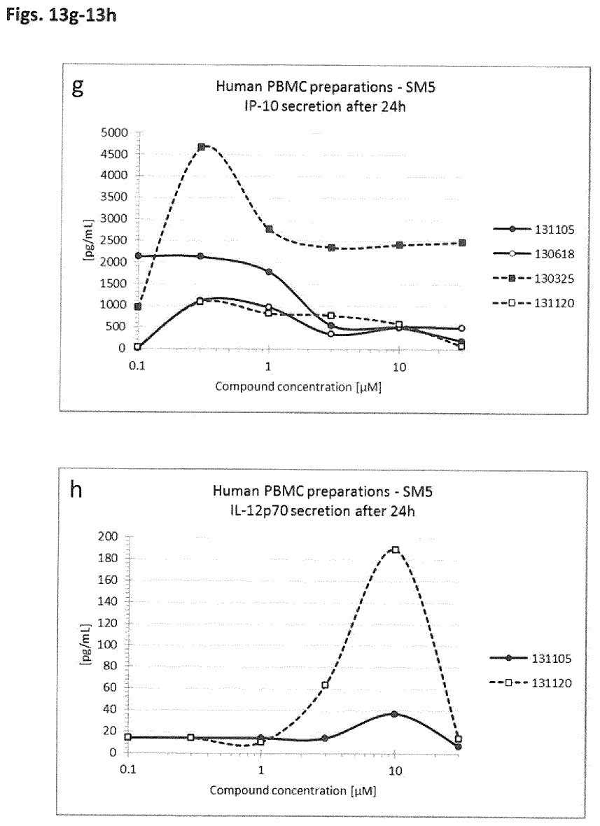

Results, which are depicted in FIGS. 12 and 13, show that exposure of human PBMCs to the TLR8 agonists resulted in secretion of the measured cytokines in a highly individualized manner.

FIG. 12 (SM4):

Agonist compound SM4 was added in an in vitro assay to freshly prepared human PBMCs from four blood donors denoted as individuals 130325, 100621, 110126, 110125 at various concentrations, i.e., 0.1 μM, 0.3 μM, 1 μM, 3 μM, 10 μM, and 30 μM. 24 hours after addition of the agonist compound, cytokine secretion into the supernatant was measured as described above. The concentrations of the various cytokines in the supernatant after 24 hour incubation with the different amounts of the agonist are shown.
FIG. 12a: Tumor necrosis factor alpha secretion from PBMCs of individuals 130325, 100621, 110126, 110125 after 24 h
FIG. 12b: Interleukin 1 beta secretion from PBMCs of individuals 130325, 100621, 110126, 110125 after 24 h
FIG. 12c: Interleukin 6 secretion from PBMCs of individuals 130325, 100621, 110126, 110125 after 24 h
FIG. 12d: Interferon gamma secretion from PBMCs of individuals 130325, 100621, 110126, 110125 after 24 h
FIG. 12e: Interleukin 10 secretion from PBMCs of individuals 130325, 100621, 110126, 1101252 after 24 h
FIG. 12f: Interferon gamma inducible protein 10 secretion from PBMCs of individuals 130325, 110126, 110125 after 24 h

FIG. 13 (SM5):

Agonist compound SM5 was added in an in vitro assay to freshly prepared human PBMCs from four blood donors denoted as individuals 131105, 130618, 130325, 131120 at various concentrations of 0.1 μM, 0.3 μM, 1 μM, 3 μM, 10 μM, and 30 μM. 24 hours after addition of the agonist compound, cytokine secretion into the supernatant was measured as described above. The concentrations of the various cytokines in the supernatant after 24 hour incubation with the different amounts of the agonist are shown.
FIG. 13a: Tumor necrosis factor alpha secretion from PBMCs of individuals 131105, 130618, 130325, 131120 after 24 h
FIG. 13b: Interleukin 1 beta secretion from PBMCs of individuals 131105, 130618, 130325, 131120 after 24 h
FIG. 13c: Interleukin 6 secretion from PBMCs of individuals 131105, 130618, 130325, 131120 after 24 h
FIG. 13d: Interleukin 8 secretion from PBMCs of individuals 131105, 131120 after 24 h
FIG. 13e: Interferon gamma secretion from PBMCs of individuals 131105, 130618, 130325, 131120 after 24 h
FIG. 13f: Interleukin 10 secretion from PBMCs of individuals 131105, 130618, 130325, 131120 after 24 h
FIG. 13g: Interferon gamma inducible protein 10 secretion from PBMCs of 131105, 130618, 130325, 131120 after 24 h
FIG. 13h: Interleukin 12p′70 secretion from PBMCs of individuals 131105, 131120 after 24 h

1. A method for determining a suitable dose of an immunotherapeutic agent for administration to an individual, comprising:
(a) separately contacting multiple different doses of the immunotherapeutic agent with immune-reactive material of the individual, and
(b) measuring at least one immunological reaction caused by the multiple different doses of the immunotherapeutic agent.

2. The method according to claim 1, wherein step (b) is characterized by qualitatively and/or quantitatively measuring at least one immunological reaction, preferably quantitatively measuring at least one immunological reaction.

3-5. (canceled)

6. The method according to claim 1, wherein the immunotherapeutic agent comprises at least one immunoreactive peptide or protein, or a nucleic acid encoding at least one immunoreactive peptide or protein.

7. The method according to claim 6, wherein the nucleic acid comprises RNA.

8-9. (canceled)

10. The method according to claim 1, wherein the multiple different doses comprise at least one dose that is below the standard dose range for the immunotherapeutic agent, or wherein the multiple different doses comprise at least one dose that lies within the standard dose range for the immunotherapeutic agent.

11. (canceled)

12. The method according to claim 1, wherein steps (a) and (b) are preformed sequentially.

13. (canceled)

14. The method according to claim 1, wherein the at least one immunological reaction comprises the production of at least one cytokine.

15. The method according to claim 14, wherein the at least one cytokine is selected from the group consisting of interleukin 6 (IL-6), tumor necrosis factor-α (TNF-α), interferon-α (IFN-α), interferon-γ (IFN-γ), interferon gamma-induced protein 10 (IP-10), interleukin-1β (IL-1β), interleukin-2 (IL-2) and interleukin-12p70 (IL-12p70).

16. (canceled)

17. The method according to claim 1, which is an in vitro method.

18-21. (canceled)

22. The method according to claim 17, wherein a dose where the at least one immunological reaction indicates an acceptable therapeutic effect for the immunotherapeutic agent, reflects a suitable dose for administration of the immunotherapeutic agent to the individual.

23. The method according to claim 1, wherein step (a) is carried out in vivo and is characterized by separately contacting multiple different doses of the immunotherapeutic agent with immune-reactive material of the individual in separate administration steps, each characterized by administration of one dose of the immunotherapeutic agent to the individual.

24. The method according to claim 23, wherein the separate administration steps are carried out subsequently and are separated from each other by time intervals of between 2 and 30 days, between 7 and 28 days, 7 days, 14 days, 21 days or 28 days.

25. The method according to claim 23, wherein measuring at least one immunological reaction is separately carried out following each separate administration step.

26. The method according to claim 23, wherein the first of the separate administration steps is characterized by administration of a dose of the immunotherapeutic agent that is below the standard dose range for the immunotherapeutic agent, and wherein the dose administered in the subsequent of the separate administration steps is optionally higher than the dose administered in the first of the separate administration steps.

27. The method according to claim 23, further comprising (c) detecting the presence or absence of at least one side effect.

28-29. (canceled)

30. The method according to claim 27, wherein where at least one side effect is detected following administration of one of the multiple different doses, all subsequent doses are administered with at least one antitoxic agent.

31. The method according to claim 30, wherein where at least one side effect is detected following administration of one of the multiple different doses that is not administered with at least one antitoxic agent, the next dose of the immunotherapeutic agent to be subsequently administered is identical to or less than the dose administered in the previous administration step.

32. The method according to claim 31, wherein the subsequent administration step that directly follows the previous administration step is further followed by one or more further administration steps that optionally represent a dose escalation scheme from step to step.

33. The method according to claim 30, wherein where at least one side effect is detected following administration of one of the multiple different doses that is administered with at least one antitoxic agent, the next dose of the immunotherapeutic agent to be subsequently administered is less than the dose administered in the previous administration step.

34-35. (canceled)

36. The method according to claim 23, wherein where no side effect is detected following administration of any of the multiple different doses, a dose where the at least one immunological reaction indicates an acceptable therapeutic effect for the immunotherapeutic agent reflects a suitable dose for administration of the immunotherapeutic agent to the individual.

37. The method according to claim 23, wherein where at least one side effect is detected following administration of any of the multiple different doses, a dose in a subsequent administration step that is administered with at least one antitoxic agent and where the at least one immunological reaction indicates an acceptable therapeutic effect for the immunotherapeutic agent reflects a suitable dose for administration of the immunotherapeutic agent to the individual.

38. The method according to claim 37, wherein where no side effect is detected following administration of any of the multiple different doses administered with at least one antitoxic agent, a dose where the at least one immunological reaction provides the strongest indication of an acceptable therapeutic effect is a suitable dose for administration of the immunotherapeutic agent to the individual.

39. The method according to claim 37, wherein where at least one side effect is detected following administration of any of the multiple different doses administered with at least one antitoxic agent, the highest dose where the side effect is not detected or is least severe or is otherwise deemed acceptable in light of the severity of the disease is a suitable dose for administration of the immunotherapeutic agent to the individual.

40-41. (canceled)

42. A method of treating an individual with a suitable dose of an immunotherapeutic agent, comprising:
a. separately contacting multiple different doses of the immunotherapeutic agent with immune-reactive material of the individual,
b. measuring at least one immunological reaction caused by the multiple different doses of the immunotherapeutic agent, wherein a dose where the at least one immunological reaction indicates an acceptable therapeutic effect for the immunotherapeutic agent reflects a suitable dose for administration to the individual, and
c. administering the immunotherapeutic agent to the individual at the suitable dose.

43. (canceled)

44. The method according to claim 42, wherein the suitable dose of the immunotherapeutic agent is administered with at least one antitoxic agent.

45. The method according to claim 42, wherein the immunotherapeutic agent is a nucleic acid encoding one or more neoepitopes.

46. (canceled)


SEQUENCE LISTING (2 sequences)

SEQ ID NO: 1 - protein, 15 aa, Artificial Sequence
  Linker sequence
  REPEAT (1)..(3): Portion of sequence repeated a times, wherein a is independently a number selected from 0, 1, 2, 3, 4, 5, 6, 7, 8, 9, 10, 11, 12, 13, 14, 15, 16, 17, 18, 19, or 20
  MISC_FEATURE (1)..(15): a + b + c + d + e are different from 0 and preferably are 2 or more, 3 or more, 4 or more or 5 or more
  REPEAT (4)..(6): Portion of sequence repeated b times, wherein b is independently a number selected from 0, 1, 2, 3, 4, 5, 6, 7, 8, 9, 10, 11, 12, 13, 14, 15, 16, 17, 18, 19, or 20
  REPEAT (7)..(9): Portion of sequence repeated c times, wherein c is independently a number selected from 0, 1, 2, 3, 4, 5, 6, 7, 8, 9, 10, 11, 12, 13, 14, 15, 16, 17, 18, 19, or 20
  REPEAT (10)..(12): Portion of sequence repeated d times, wherein d is independently a number selected from 0, 1, 2, 3, 4, 5, 6, 7, 8, 9, 10, 11, 12, 13, 14, 15, 16, 17, 18, 19, or 20
  REPEAT (13)..(15): Portion of sequence repeated e times, wherein e is independently a number selected from 0, 1, 2, 3, 4, 5, 6, 7, 8, 9, 10, 11, 12, 13, 14, 15, 16, 17, 18, 19, or 20
GGSGSSGGGSSGGSG

SEQ ID NO: 2 - protein, 9 aa, Artificial Sequence
  linker sequence
GGSGGGGSG